(19) United States
(12) Patent Application Publication
(10) Pub. No.: US 2020/0123137 A1
(43) Pub. Date: Apr. 23, 2020

(54) DEUTERATED CFTR MODULATORS AND METHODS OF USE

(71) Applicants: ABBVIE S.À.R.L., Luxembourg (LU); GALAPAGOS NV, Mechelen (BE)

(72) Inventors: Vincent S. Chan, Evanston, IL (US); Philip R. Kym, Libertyville, IL (US); Shashank Shekhar, Vernon Hills, IL (US); Xueqing Wang, Northbrook, IL (US)

(73) Assignees: ABBVIE S.À.R.L., Luxembourg (LU); GALAPAGOS NV, Mechelen (BE)

(21) Appl. No.: 16/471,762

(22) PCT Filed: Dec. 19, 2017
(86) PCT No.: PCT/IB2017/058179

§ 371 Date: Jun. 20, 2019

Related U.S. Application Data
(60) Provisional application No. 62/436,673, filed on Dec. 20, 2016.

(30) Foreign Application Priority Data
Dec. 19, 2017 (IB) PCT/IB2017/058179

(51) Int. Cl. C07D 407/12; A61P 11/12

(52) U.S. Cl. CPC C07D 407/12; A61K 45/06; C07B 2200/05; A61P 11/12

(57) ABSTRACT

The invention discloses compounds of Formula (I)

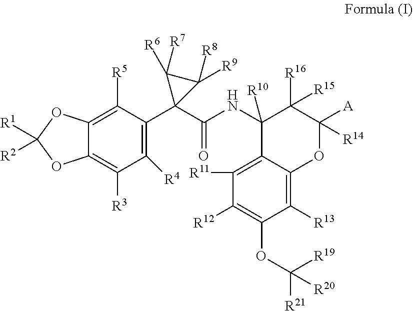

wherein R¹, R², R³, R⁴, R⁵, R⁶, R⁷, R⁸, R⁹, R¹⁰, R¹¹, R¹², R¹³, R¹⁴, R¹⁵, R¹⁶, A, R¹⁹, R²⁰, and R²¹ are as defined herein. The present invention relates to deuterated compounds and their use in the treatment of cystic fibrosis, methods for their production, pharmaceutical compositions comprising the same, and methods of treating cystic fibrosis by administering a compound of the invention.

### CROSS REFERENCE TO RELATED APPLICATIONS

This application claims priority to U.S. Provisional Application No. 62/436,673, filed Dec. 20, 2016, which is incorporated herein by its entirety for all purposes.

### BACKGROUND OF THE INVENTION

### Technical Field

The invention relates to deuterated modulators of the Cystic Fibrosis Transmembrane Conductance Regulator (CFTR) protein, useful in treating diseases and conditions mediated and modulated by CFTR. The invention also relates to compositions containing compounds of the invention, processes for their preparation, and methods of treatment using them.

### Description of Related Technology

ABC transporters are a family of homologous membrane transporter proteins regulating the transport of a wide variety of pharmacological agents (for example drugs, xenobiotics, anions, etc.) that bind and use cellular adenosine triphosphate (ATP) for their specific activities. Some of these transporters were found to defend malignant cancer cells against chemotherapeutic agents, acting as multidrug resistance proteins (like the MDR1-P glycoprotein, or the multidrug resistance protein, MRP 1). So far, 48 ABC transporters, grouped into 7 families based on their sequence identity and function, have been identified.

ABC transporters provide protection against harmful environmental compounds by regulating a variety of important physiological roles within the body, and therefore represent important potential drug targets for the treatment of diseases associated with transporter defects, outwards cell drug transport, and other diseases in which modulation of ABC transporter activity may be beneficial.

The cAMP/ATP-mediated anion channel, CFTR, is one member of the ABC transporter family commonly associated with diseases, which is expressed in a variety of cell types, including absorptive and secretory epithelia cells, where it regulates anion flux across the membrane, as well as the activity of other ion channels and proteins. The activity of CFTR in epithelial cells is essential for the maintenance of electrolyte transport throughout the body, including respiratory and digestive tissue (Quinton, P. M., 1990. Cystic fibrosis: a disease in electrolyte transport. FASEB J. 4, 2709-2717).

The gene encoding CFTR has been identified and sequenced (Kerem, B., Rommens, J. M., Buchanan, J. A., Markiewicz, D., Cox, T. K., Chakravarti, A., Buchwald, M., Tsui, L. C., 1989. Identification of the cystic fibrosis gene: genetic analysis. Science 245, 1073-1080). CFTR comprises about 1480 amino acids that encode a protein made up of a tandem repeat of transmembrane domains, each containing six transmembrane helices and a nucleotide binding domain. The pair of transmembrane domains is linked by a large, polar, regulatory (R)-domain with multiple phosphorylation sites that regulate channel activity and cellular trafficking.

Cystic fibrosis (CF) is caused by a defect in this gene which induces mutations in CFTR. Cystic fibrosis is the most common fatal genetic disease in humans, and affects ˜0.04% of white individuals (Bobadilla, J. L., Macek, M., Jr, Fine, J. P., Farrell, P. M., 2002. Cystic fibrosis: a worldwide analysis of CFTR mutations—correlation with incidence data and application to screening. Hum. Mutat. 19, 575-606. doi:10.1002/humu.10041), for example, in the United States, about one in every 2,500 infants is affected, and up to 10 million people carry a single copy of the defective gene without apparent ill effects; moreover subjects bearing a single copy of the gene exhibit increased resistance to cholera and to dehydration resulting from diarrhea. This effect might explain the relatively high frequency of the CF gene within the population.

In contrast, individuals with two copies of the CF associated gene suffer from the debilitating and fatal effects of CF, including chronic lung infections.

In cystic fibrosis patients, mutations in endogenous respiratory epithelial CFTR fails to confer chloride and bicarbonate permeability to epithelial cells in lung and other tissues, thus leading to reduced apical anion secretion and disruptions of the ion and fluid transport. This decrease in anion transport causes an enhanced mucus and pathogenic agent accumulation in the lung triggering microbial infections that ultimately cause death in CF patients.

Beyond respiratory disease, CF patients also suffer from gastrointestinal problems and pancreatic insufficiency that result in death if left untreated. Furthermore, female subjects with cystic fibrosis suffer from decreased fertility, whilst males with cystic fibrosis are infertile.

A variety of disease causing mutations has been identified through sequence analysis of the CFTR gene of CF chromosomes (Kerem, B., Rommens, J. M., Buchanan, J. A., Markiewicz, D., Cox, T. K., Chakravarti, A., Buchwald, M., Tsui, L. C., 1989. Identification of the cystic fibrosis gene: genetic analysis. Science 245, 1073-1080). ΔF508-CFTR, the most common CF mutation (present in at least 1 allele in ˜90% of CF patients) and occurring in approximately 70% of the cases of cystic fibrosis, contains a single amino acid deletion of phenylalanine 508. This deletion prevents the nascent protein from folding correctly, which protein in turn cannot exit the endoplasmic reticulum (ER) and traffic to the plasma membrane, and then is rapidly degraded. As a result, the number of channels present in the membrane is far less than in cells expressing wild-type CFTR. In addition to impaired trafficking, the mutation results in defective channel gating. Indeed, even if ΔF508-CFTR is allowed to reach the cell plasma membrane by low-temperature (27° C.) rescue where it can function as a cAMP-activated chloride channel, its activity is decreased significantly compared with WT-CFTR (Pasyk, E. A., Foskett, J. K., 1995. Mutant (6F508) Cystic Fibrosis Transmembrane Conductance Regulator Cl⁻ Channel Is Functional When Retained in Endoplasmic Reticulum of Mammalian Cells. J. Biol. Chem. 270, 12347-12350).

Other mutations with lower incidence have also been identified that alter the channel regulation or the channel conductance. In case of the channel regulation mutants, the mutated protein is properly trafficked and localized to the plasma membrane but either cannot be activated or cannot function as a chloride channel (e.g. missense mutations located within the nucleotide binding domains), examples of these mutations are G551D, G178R, and G1349D. Mutations affecting chloride conductance have a CFTR protein that is correctly trafficked to the cell membrane but that generates reduced chloride flow (e.g. missense mutations located within the membrane-spanning domain), examples of these mutations are R117H and R334W.

In addition to cystic fibrosis, CFTR activity modulation may be beneficial for other diseases not directly caused by mutations in CFTR, such as, for example, chronic obstructive pulmonary disease (COPD), dry eye disease, and Sjögren's syndrome.

COPD is characterized by a progressive and non-reversible airflow limitation, which is due to mucus hypersecretion, bronchiolitis, and emphysema. A potential treatment of mucus hypersecretion and impaired mucociliary clearance that is common in COPD could consist in using activators of mutant or wild-type CFTR. In particular, the anion secretion increase across CFTR may facilitate fluid transport into the airway surface liquid to hydrate the mucus and optimize periciliary fluid viscosity. The resulting enhanced mucociliary clearance would help in reducing the symptoms associated with COPD.

Dry eye disease is characterized by a decrease in tear production and abnormal tear film lipid, protein and mucin profiles. Many factors may cause dry eye disease, some of which include age, arthritis, Lasik eye surgery, chemical/thermal burns, medications, allergies, and diseases, such as cystic fibrosis and Sjögren's syndrome. Increasing anion secretion via CFTR could enhance fluid transport from the corneal endothelial cells and secretory glands surrounding the eye, and eventually improve corneal hydration, thus helping to alleviate dry eye disease associated symptoms. Sjögren's syndrome is an autoimmune disease where the immune system harms moisture-producing glands throughout the body, including the eye, mouth, skin, respiratory tissue, liver, vagina, and gut. The ensuing symptoms, include, dry eye, mouth, and vagina, as well as lung disease. Sjögren's syndrome is also associated with rheumatoid arthritis, systemic lupus, systemic sclerosis, and polymyositis/dermatomyositis. The cause of the disease is believed to lie in defective protein trafficking, for which treatment options are limited. As a consequence, modulation of CFTR activity may help hydrating the various organs and help to elevate the associated symptoms.

In addition to CF, the defective protein trafficking induced by the ΔF508-CFTR has been shown to be the underlying basis for a wide range of other diseases, in particular diseases where the defective functioning of the endoplasmic reticulum (ER) may either prevent the CFTR protein to exit the cell, and/or the misfolded protein is degraded (Morello, J.-P., Bouvier, M., Petaja-Repo, U. E., Bichet, D. G., 2000. Pharmacological chaperones: a new twist on receptor folding. Trends Pharmacol. Sci. 21, 466-469. doi: 10.1016/S0165-6147(00)01575-3; Shastry, B. S., 2003. Neurodegenerative disorders of protein aggregation. Neurochem. Int. 43, 1-7. doi:10.1016/S0197-0186(02)00196-1; Zhang, W., Fujii, N., Naren, A. P., 2012. Recent advances and new perspectives in targeting CFTR for therapy of cystic fibrosis and enterotoxin-induced secretory diarrheas. Future Med. Chem. 4, 329-345. doi: 10.4155/fmc. 12.1).

A number of genetic diseases are associated with a defective ER processing equivalent to the defect observed with CFTR in CF such as glycanosis CDG type 1, hereditary emphysema (α-1-antitrypsin (PiZ variant)), congenital hyperthyroidism, osteogenesis imperfecta (Type I, II, or IV procollagen), hereditary hypofibrinogenemia (fibrinogen), ACT deficiency (α-1-antichymotrypsin), diabetes insipidus (DI), neurohypophyseal DI (vasopressin hormoneN2-receptor), nephrogenic DI (aquaporin II), Charcot-Marie Tooth syndrome (peripheral myelin protein 22), Pelizaeus-Merzbacher disease, neurodegenerative diseases such as Alzheimer's disease (APP and presenilins), Parkinson's disease, amyotrophic lateral sclerosis, progressive supranuclear palsy, Pick's disease, several polyglutamine neurological disorders such as Huntington's disease, spinocerebellar ataxia type I, spinal and bulbar muscular atrophy, dentatorubral pallidoluysian, and myotonic dystrophy, as well as spongiform encephalopathies, such as hereditary Creutzfeldt-Jakob disease (prion protein processing defect), Fabry disease (lysosomal a-galactosidase A), Straussler-Scheinker syndrome, chronic obstructive pulmonary disease (COPD), dry eye disease, and Sjögren's syndrome.

In addition to up-regulation of the activity of CFTR, anion secretion reduction by CFTR modulators may be beneficial for the treatment of secretory diarrheas, in which epithelial water transport is dramatically increased as a result of secretagogue activated chloride transport. The mechanism involves elevation of cAMP and stimulation of CFTR.

Regardless of the cause, excessive chloride transport is seen in all diarrheas, and results in dehydration, acidosis, impaired growth and death. Acute and chronic diarrheas remain a major medical problem worldwide, and are a significant factor in malnutrition, leading to death in children of less than five years old (5,000,000 deaths/year). Furthermore, in patients with chronic inflammatory bowel disease (IBD) and/or acquired immunodeficiency syndrome (AIDS), diarrhea is a dangerous condition.

Substitution of hydrogen with heavier isotopes such as deuterium, i.e. ²H, may afford certain therapeutic advantages resulting from greater metabolic stability, for example, increased in vivo half-life or reduced dosage requirements, and hence may be preferred in some circumstances. It has also been proposed that deuterium incorporation may have benefits in reducing interpatient variability, adverse events, and genotoxicity (Gant, Thomas G, 2014. Using Deuterium in Drug Discovery: Leaving the Label in the Drug. Journal of Medicinal Chemistry 57.9, 2014, 3595-611). Accordingly, there is a need for novel deuterated compounds able to modulate CFTR. In particular, the present invention discloses deuterated compounds that may act as CFTR modulators for the treatment of cystic fibrosis. The present invention also provides methods for the preparation of these compounds, pharmaceutical compositions comprising these compounds and methods for the treatment of cystic fibrosis by administering the compounds of the invention.

### SUMMARY

In one aspect, the invention provides for compounds of Formula (I), or a pharmaceutically acceptable salt thereof,

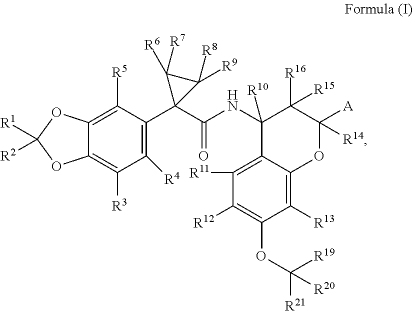

wherein
R¹ and R² are each independently hydrogen, deuterium, or fluorine;
A is

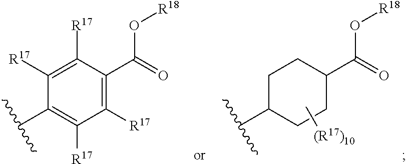

R³, R⁴, R⁵, R⁶, R⁷, R⁸, R⁹, R¹⁰, R¹¹, R¹², R¹³, R¹⁴, R¹⁵, R¹⁶, and R¹⁸ are each independently hydrogen or deuterium;
R¹⁷, at each occurrence, is independently hydrogen or deuterium; and
R¹⁹, R²⁰, and R²¹ are each independently hydrogen, deuterium, or fluorine;
provided that, at least one of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ is deuterium.

Another aspect of the invention relates to pharmaceutical compositions comprising a compound of the invention, and a pharmaceutical carrier. Such compositions can be administered in accordance with a method of the invention, typically as part of a therapeutic regimen for treatment or prevention of conditions and disorders related to Cystic Fibrosis Transmembrane Conductance Regulator activity. In a particular aspect, the pharmaceutical compositions may additionally comprise further therapeutically active ingredients suitable for use in combination with the compounds of the invention. In a more particular aspect, the further therapeutically active ingredient is an agent for the treatment of cystic fibrosis.

Moreover, the compounds of the invention, useful in the pharmaceutical compositions and treatment methods disclosed herein, are pharmaceutically acceptable as prepared and used.

Yet another aspect of the invention relates to a method for treating, or preventing conditions and disorders related to Cystic Fibrosis Transmembrane Conductance Regulator activity in mammals. More particularly, the method is useful for treating or preventing conditions and disorders related to cystic fibrosis, Sjögren's syndrome, pancreatic insufficiency, chronic obstructive lung disease, or chronic obstructive airway disease. Accordingly, the compounds and compositions of the invention are useful as a medicament for treating or preventing Cystic Fibrosis Transmembrane Conductance Regulator modulated disease.

The compounds, compositions comprising the compounds, methods for making the compounds, and methods for treating or preventing conditions and disorders by administering the compounds are further described herein.

In a particular aspect, the compounds of the invention are provided for use in the treatment of cystic fibrosis. In a particular aspect, the compounds of the invention are provided for use in the treatment of cystic fibrosis caused by class I, II, III, IV, V, and/or VI mutations.

The present invention also provides pharmaceutical compositions comprising a compound of the invention, and a suitable pharmaceutical carrier for use in medicine. In a particular aspect, the pharmaceutical composition is for use in the treatment of cystic fibrosis.

These and other objects of the invention are described in the following paragraphs. These objects should not be deemed to narrow the scope of the invention.

### DETAILED DESCRIPTION OF THE INVENTION

In one aspect, the invention provides for compounds of Formula (I), or a pharmaceutically acceptable salt thereof,

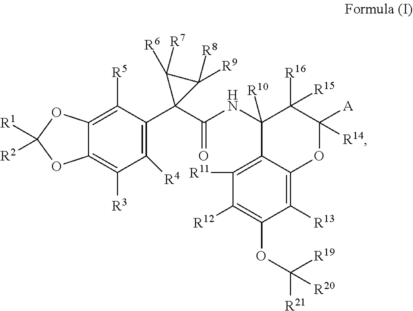

wherein
R¹, R², R³, R⁴, R⁵, R⁶, R⁷, R⁸, R⁹, R¹⁰, R¹¹, R¹², R¹³, R¹⁴, R¹⁵, R¹⁶, A, R¹⁹, R²⁰, and R²¹ are defined above in the Summary and below in the Detailed Description. Further, compositions comprising such compounds and methods for treating conditions and disorders using such compounds and compositions are also included.

Compounds included herein may contain one or more variable(s) that occur more than one time in any substituent or in the formulae herein. Definition of a variable on each occurrence is independent of its definition at another occurrence. Further, combinations of substituents are permissible only if such combinations result in stable compounds. Stable compounds are compounds which can be isolated from a reaction mixture.

### Definitions

It is noted that, as used in this specification and the intended claims, the singular form “a,” “an,” and “the” include plural referents unless the context clearly dictates otherwise. Thus, for example, reference to “a compound” includes a single compound as well as one or more of the same or different compounds; reference to “a pharmaceutically acceptable carrier” means a single pharmaceutically acceptable carrier as well as one or more pharmaceutically acceptable carriers, and the like.

As used in the specification and the appended claims, unless specified to the contrary, the following terms have the meaning indicated:

The term “isotopic enrichment factor” refers to the ratio of the amount of a particular isotope in an enriched compound to the known natural amount of the isotope. Therefore, isotopic enrichment factor can be described as % deuterium/0.0156 (natural abundance). For example, a compound with 50% deuterium at a specific position would have an isotopic enrichment factor of 50/0.0156=3205.

The term “isotopologue” refers to a compound with an identical chemical structure, but which differs in isotopic composition.

The terms “treat”, “treating”, and “treatment” refer to a method of alleviating or abrogating a disease and/or its attendant symptoms. In certain embodiments, “treat,” “treating,” and “treatment” refer to ameliorating at least one physical parameter, which may not be discernible by the subject. In yet another embodiment, “treat”, “treating”, and “treatment” refer to modulating the disease or disorder, either physically (for example, stabilization of a discernible symptom), physiologically (for example, stabilization of a physical parameter), or both. In a further embodiment, “treat”, “treating”, and “treatment” refer to slowing the progression of the disease or disorder.

The terms “prevent”, “preventing”, and “prevention” refer to a method of preventing the onset of a disease and/or its attendant symptoms or barring a subject from acquiring a disease. As used herein, “prevent”, “preventing” and “prevention” also include delaying the onset of a disease and/or its attendant symptoms and reducing a subject's risk of acquiring or developing a disease or disorder.

The phrase “therapeutically effective amount” means an amount of a compound, or a pharmaceutically acceptable salt thereof, sufficient to prevent the development of or to alleviate to some extent one or more of the symptoms of the condition or disorder being treated when administered alone or in conjunction with another therapeutic agent for treatment in a particular subject or subject population. The “therapeutically effective amount” may vary depending on the compound, the disease and its severity, and the age, weight, health, etc., of the subject to be treated. For example in a human or other mammal, a therapeutically effective amount may be determined experimentally in a laboratory or clinical setting, or may be the amount required by the guidelines of the United States Food and Drug Administration, or equivalent foreign agency, for the particular disease and subject being treated.

The term “subject” is defined herein to refer to animals such as mammals, including, but not limited to, primates (e.g., humans), cows, sheep, goats, pigs, horses, dogs, cats, rabbits, rats, mice and the like. In one embodiment, the subject is a human. The terms “human,” “patient,” and “subject” are used interchangeably herein.

As used herein, “Class I mutation(s)” refers to mutations which interfere with protein synthesis. They result in the introduction of a premature signal of termination of translation (stop codon) in the mRNA. The truncated CFTR proteins are unstable and rapidly degraded, so, the net effect is that there is no protein at the apical membrane. In particular, Class I mutation(s) refers to p.Gly542X (G542X), W1282X, c.489+1G>T (621+1G>T), or c.579+1G>T (711+1G>T) mutation. More particularly, Class I mutation(s) refers to G542X; or W1282X mutations.

As used herein, “Class II mutation(s)” refers to mutations which affect protein maturation. These lead to the production of a CFTR protein that cannot be correctly folded and/or trafficked to its site of function on the apical membrane. In particular, Class II mutation(s) refers to p.Phe508del (F508del), p.Ile507del, or p.Asn1303Lys (N1303K) mutations. More particularly, Class II mutation(s) refers to F508del or N1303K mutations.

As used herein, “Class III mutation(s)” refers to mutations which alter the regulation of the CFTR channel. The mutated CFTR protein is properly trafficked and localized to the plasma membrane but cannot be activated, or it cannot function as a chloride channel. In particular, Class III mutation(s) refers to p.Gly551Asp (G551D), G551S, R553G, G1349D, S1251N, G178R, S549N mutations. More particularly, Class III mutation(s) refers to G551D, R553G, G1349D, S1251N, G178R, or S549N mutations.

As used herein, “Class IV mutation(s)” refers to mutations which affect chloride conductance. The CFTR protein is correctly trafficked to the cell membrane but generates reduced chloride flow or a “gating defect” (most are missense mutations located within the membrane-spanning domain). In particular, Class IV mutation(s) refers to p.Arg117His (R117H), R347P, or p.Arg334Trp (R334W) mutations.

As used herein, “Class V mutation(s)” refers to mutations which reduce the level of normally functioning CFTR at the apical membrane or result in a “conductance defect” (for example partially aberrant splicing mutations or inefficient trafficking missense mutations). In particular, Class V mutation(s) refers to c.1210-12T[5] (5T allele), c.S3140-26A>G (3272-26A>G), c.3850-2477C>T (3849+10kbC>T) mutations.

As used herein, “Class VI mutation(s)” refers to mutations which decrease the stability of the CFTR which is present or which affect the regulation of other channels, resulting in inherent instability of the CFTR protein. In effect, although functional, the CFTR protein is unstable at the cell surface and it is rapidly removed and degraded by cell machinery. In particular, Class VI mutation(s) refers to Rescued F508del, 120del23, N287Y, 4326dellTC, or 4279insA mutations. More particularly, Class VI mutation(s) refers to Rescued F508del mutations.

### Compounds

Compounds of the invention have the general Formula (I) as described above.

Particular values of variable groups are as follows. Such values may be used where appropriate with any of the other values, definitions, claims or embodiments defined hereinbefore or hereinafter.

### Formula (I)

One embodiment pertains to compounds of Formula (I), or pharmaceutically acceptable salts thereof,

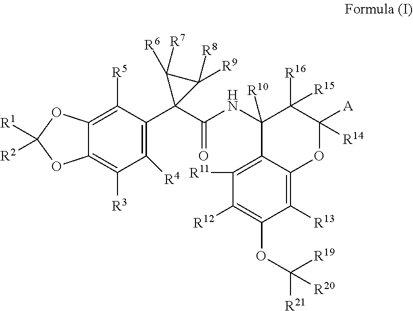

wherein
R¹ and R² are each independently hydrogen, deuterium, or fluorine;
A is

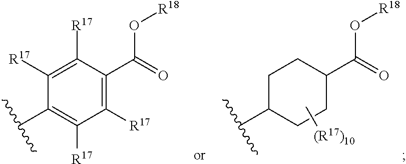

R³, R⁴, R⁵, R⁶, R⁷, R⁸, R⁹, R¹⁰, R¹¹, R¹², R¹³, R¹⁴, R¹⁵, R¹⁶, and R¹⁸ are each independently hydrogen or deuterium;
R¹⁷, at each occurrence, is independently hydrogen or deuterium; and
R¹⁹, R²⁰, and R²¹ are each independently hydrogen, deuterium, or fluorine;
provided that, at least one of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ is deuterium.

In one embodiment of Formula (I), one of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ is deuterium. In another embodiment of Formula (I), two of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ are deuterium. In another embodiment of Formula (I), three of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ are deuterium. In another embodiment of Formula (I), four of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ are deuterium. In another embodiment of Formula (I), five of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ are deuterium. In another embodiment of Formula (I), six of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ are deuterium. In another embodiment of Formula (I), seven of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ are deuterium. In another embodiment of Formula (I), eight of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ are deuterium. In another embodiment of Formula (I), nine of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ are deuterium. In another embodiment of Formula (I), ten of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ are deuterium. In another embodiment of Formula (I), eleven of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ are deuterium. In another embodiment of Formula (I), twelve of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ are deuterium. In another embodiment of Formula (I), thirteen of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ are deuterium. In another embodiment of Formula (I), fourteen of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ are deuterium. In another embodiment of Formula (I), fifteen of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ are deuterium. In another embodiment of Formula (I), sixteen of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ are deuterium. In another embodiment of Formula (I), seventeen of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ are deuterium. In another embodiment of Formula (I), eighteen of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ are deuterium. In another embodiment of Formula (I), nineteen of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ are deuterium. In another embodiment of Formula (I), twenty of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ are deuterium. In another embodiment of Formula (I), twenty-one of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ are deuterium. In another embodiment of Formula (I), twenty-two of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ are deuterium. In another embodiment of Formula (I), twenty-three of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ are deuterium. In another embodiment of Formula (I), twenty-four of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ are deuterium. In another embodiment of Formula (I), twenty-five of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ are deuterium. In another embodiment of Formula (I), twenty-six of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ are deuterium. In another embodiment of Formula (I), twenty-seven of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ are deuterium. In another embodiment of Formula (I), twenty-eight of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ are deuterium. In another embodiment of Formula (I), twenty-nine of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ are deuterium. In another embodiment of Formula (I), thirty of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ are deuterium.

In one embodiment of Formula (I), R¹ and R² are each independently hydrogen, deuterium, or fluorine. In another embodiment of Formula (I), one of R¹ and R² is deuterium, and the other is hydrogen or fluorine. In another embodiment of Formula (I), R¹ and R² are each hydrogen, provided that at least one of R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ is deuterium. In another embodiment of Formula (I), R¹ and R² are each deuterium. In another embodiment of Formula (I), R¹ and R² are each fluorine, provided that at least one of R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ is deuterium.

In one embodiment of Formula (I), R³, R⁴, and R⁵, are each independently hydrogen or deuterium. In another embodiment of Formula (I), R³, R⁴, and R⁵, are each hydrogen provided that at least one of R¹, R², R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ is deuterium. In another embodiment of Formula (I), one of R³, R⁴, and R⁵ is independently deuterium. In another embodiment of Formula (I), two of R³, R⁴, and R⁵ are deuterium. In another embodiment of Formula (I), R³, R⁴, and R⁵ are each deuterium.

In one embodiment of Formula (I), R⁶, R⁷, R⁸, and R⁹, are each independently hydrogen or deuterium. In another embodiment of Formula (I), R⁶, R⁷, R⁸, and R⁹, are each hydrogen provided that at least one of R¹, R², R³, R⁴, R⁵, R¹⁰, R¹¹, R¹², R¹³, R¹⁴, R¹⁵, R¹⁶, R¹⁷, R¹⁸, R¹⁹, R²⁰, and R²¹ is deuterium. In one embodiment of Formula (I), one of R⁶, R⁷, R⁸, and R⁹, is deuterium. In another embodiment of Formula (I), two of R⁶, R⁷, R⁸, and R⁹, are deuterium. In another embodiment of Formula (I), three of R⁶, R⁷, R⁸, and R⁹, are deuterium. In another embodiment of Formula (I), R⁶, R⁷, R⁸, and R⁹ are each deuterium.

In one embodiment of Formula (I), R¹⁰, R¹⁴, R¹⁵, and R¹⁶, are each independently hydrogen or deuterium. In another embodiment of Formula (I), R¹⁰, R¹⁴, R¹⁵, and R¹⁶, are each hydrogen provided that at least one of R¹, R², R³, R⁴, R⁵, R⁶, R⁷, R⁸, R⁹, R¹¹, R¹², R¹³, R¹⁷, R¹⁸, R¹⁹, R²⁰, and R²¹ is deuterium. In one embodiment of Formula (I), one of R¹⁰, R¹⁴, R¹⁵, and R¹⁶, is deuterium. In another embodiment of Formula (I), two of R¹⁰, R¹⁴, R¹⁵, and R¹⁶, are deuterium. In another embodiment of Formula (I), three of R¹⁰, R¹⁴, R¹⁵, and R¹⁶, are deuterium. In another embodiment of Formula (I), R¹⁰, R¹⁴, R¹⁵, and R¹⁶, are deuterium.

In one embodiment of Formula (I), R¹¹, R¹², and R¹³ are each independently hydrogen or deuterium. In another embodiment of Formula (I), R¹¹, R¹², and R¹³ are each hydrogen provided that at least one of R¹, R², R³, R⁴, R⁵, R⁶, R⁷, R⁸, R⁹, R¹⁰, R¹⁴, R¹⁵, R¹⁶, R¹⁷, R¹⁸, R¹⁹, R²⁰, and R²¹ is deuterium. In one embodiment of Formula (I), one of R¹¹, R¹², and R¹³ is deuterium. In another embodiment of Formula (I), two of R¹¹, R¹², and R¹³ are deuterium. In another embodiment of Formula (I), R¹¹, R¹², and R¹³ are deuterium.

In one embodiment of Formula (I), R¹⁹, R²⁰, and R²¹ are each independently hydrogen, deuterium, or fluorine. In another embodiment of Formula (I), R¹⁹, R²⁰, and R²¹ are each hydrogen, provided that at least one of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, and R¹⁸ is deuterium. In another embodiment of Formula (I), R¹⁹, R²⁰, and R²¹ are each fluorine provided that at least one of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, and R¹⁸ is deuterium. In another embodiment of Formula (I), one of R¹⁹, R²⁰, and R²¹ is fluorine provided that at least one of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, and R¹⁸ is deuterium. In another embodiment of Formula (I), two of R¹⁹, R²⁰, and R²¹ are fluorine provided that at least one of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, and R¹⁸ is deuterium. In another embodiment of Formula (I), one of R¹⁹, R²⁰, and R²¹ is deuterium or fluorine. In another embodiment of Formula (I), two of R¹⁹, R²⁰, and R²¹ are deuterium or fluorine. In another embodiment of Formula (I), R¹⁹, R²⁰, and R²¹ are deuterium.

In one embodiment of Formula (I), R¹⁸ is hydrogen or deuterium. In another embodiment of Formula (I), R¹⁸ is hydrogen provided that at least one of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁹, R²⁰, and R²¹ is deuterium. In another embodiment of Formula (I), R¹⁸ is deuterium.

In one embodiment of Formula (I), A is

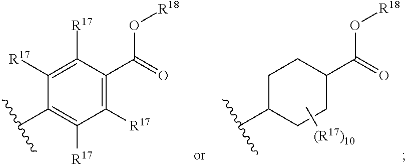

R¹⁸ is hydrogen or deuterium; and R¹⁷, at each occurrence is independently hydrogen or deuterium.

In one embodiment of Formula (I), A is

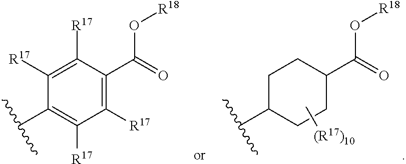

In another embodiment of Formula (I), A is

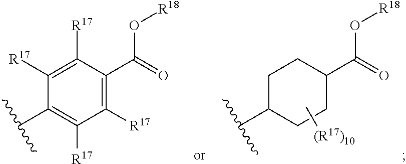

and R¹⁸ is hydrogen or deuterium. In another embodiment of Formula (I), A is

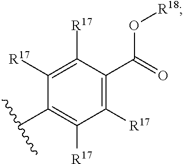

and R¹⁸ is hydrogen or deuterium. In another embodiment of Formula (I), A is

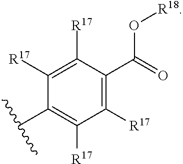

In another embodiment of Formula (I), A is

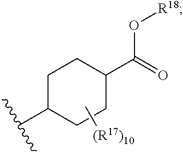

and R¹⁸ is hydrogen or deuterium. In another embodiment of Formula (I), A is

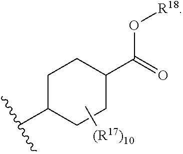

In another embodiment of Formula (I), A is H

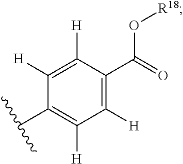

provided that that at least one of R¹, R², R³, R⁴, R⁵, R⁶, R⁷, R⁸, R⁹, R¹⁰, R¹¹, R¹², R¹³, R¹⁴, R¹⁵, R¹⁶, R¹⁸, R¹⁹, R²⁰, and R²¹ is deuterium. In another embodiment of Formula (I), A is

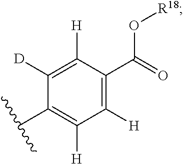

and R¹⁸ is hydrogen or deuterium. In another embodiment of Formula (I), A is

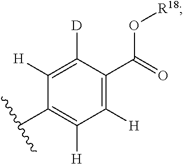

and R¹⁸ is hydrogen or deuterium. In another embodiment of Formula (I), A is

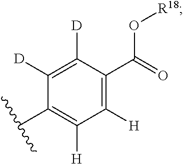

and R¹⁸ is hydrogen or deuterium. In another embodiment of Formula (I), A is

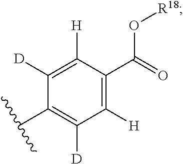

and R¹⁸ is hydrogen or deuterium. In another embodiment of Formula (I), A is

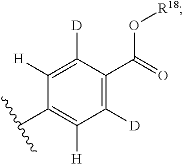

and R¹⁸ is hydrogen or deuterium. In another embodiment of Formula (I), A is

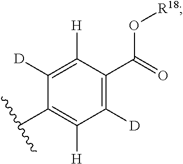

and R¹⁸ is hydrogen or deuterium. In another embodiment of Formula (I), A is

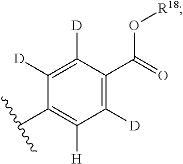

and R¹⁸ is hydrogen or deuterium. In another embodiment of Formula (I), A is

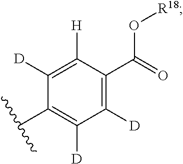

and R¹⁸ is hydrogen or deuterium. In another embodiment of Formula (I), A is

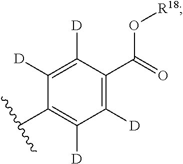

and R¹⁸ is hydrogen or deuterium.

In one embodiment of Formula (I), A is

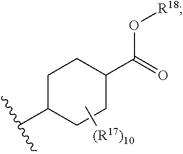

and R¹⁸ is hydrogen or deuterium. In another embodiment of Formula (I), A is

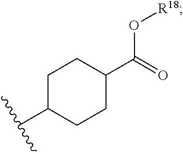

provided that that at least one of R¹, R², R³, R⁴, R⁵, R⁶, R⁷, R⁸, R⁹, R¹⁰, RR, R¹², R¹³, R¹⁴, R¹⁵, R¹⁶, R¹⁸, R¹⁹, R²⁰, and R²¹ is deuterium. In another embodiment of Formula (I), A is

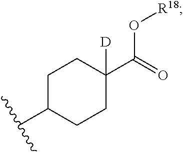

and R¹⁸ is hydrogen or deuterium. In another embodiment of Formula (I), A is

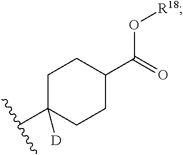

and R¹⁸ is hydrogen or deuterium. In another embodiment of Formula (I), A is

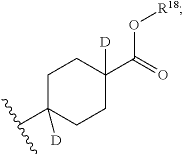

and R¹⁸ is hydrogen or deuterium. In another embodiment of Formula (I), A is

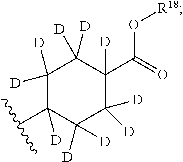

and R¹⁸ is hydrogen or deuterium.

One embodiment pertains to 4-[(2R,4R)-4-({[1-(2,2-difluoro-1,3-benzodioxol-5-yl)cyclopropyl]carbonyl}amino)-7-(difluoromethoxy)(3,3,4,5,6-²H₅)-3,4-dihydro-2H-chromen-2-yl](3,5-²H₂)benzoic acid. Another embodiment pertains to 4-[(2R,4R)-4-({[1-(2,2-difluoro-1,3-benzodioxol-5-yl)(2H₄)cyclopropyl]carbonyl}amino)-7-(difluoromethoxy)-3,4-dihydro-2H-chromen-2-yl]benzoic acid. Another embodiment pertains to 4-[(2R,4R)-4-({[1-(2,2-difluoro-1,3-benzodioxol-5-yl)cyclopropyl]carbonyl}amino)-7-(difluoromethoxy)(3,3,4,5,6-²H₅)-3,4-dihydro-2H-chromen-2-yl](3,5-²H₂)benzoic acid, 4-[(2R,4R)-4-({[1-(2,2-difluoro-1,3-benzodioxol-5-yl)(²H₄)cyclopropyl]carbonyl}amino)-7-(difluoromethoxy)-3,4-dihydro-2H-chromen-2-yl]benzoic acid, or a pharmaceutically acceptable salt thereof.

### Formula (II)

One embodiment pertains to compounds of Formula (II), or pharmaceutically acceptable salts thereof,

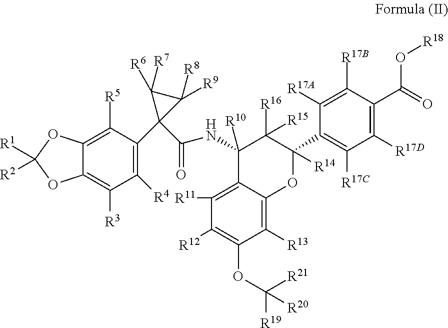

wherein
R¹ and R² are each independently hydrogen, deuterium, or fluorine;
R³, R⁴, R⁵, R⁶, R⁷, R⁸, R⁹, R¹⁰, R¹¹, R¹², R¹³, R¹⁴, R¹⁵, R¹⁶, and R¹⁸ are each independently hydrogen or deuterium;
R^{17A}, R^{17B}, R^{17C}, R^{17D} are each independently hydrogen or deuterium; and
R¹⁹, R²⁰, and R²¹ are each independently hydrogen, deuterium, or fluorine;
provided that, at least one of R¹, R², R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, R¹⁹, R²⁰, and R²¹ is deuterium.

In one embodiment of Formula (II), one of R¹, R², R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, R¹⁹, R²⁰, and R²¹ is deuterium. In another embodiment of Formula (II), two of R¹, R², R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, R¹⁹, R²⁰, and R²¹ are deuterium. In another embodiment of Formula (II), three of R¹, R², R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, R¹⁹, R²⁰, and R²¹ are deuterium. In another embodiment of Formula (II), four of R¹, R², R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, R¹⁹, R²⁰, and R²¹ are deuterium. In another embodiment of Formula (II), five of R¹, R², R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, R¹⁹, R²⁰, and R²¹ are deuterium. In another embodiment of Formula (II), six of R¹, R², R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, R¹⁹, R²⁰, and R²¹ are deuterium. In another embodiment of Formula (II), seven of R¹, R², R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17D}, R¹⁸, R¹⁹, R²⁰, and R²¹ are deuterium. In another embodiment of Formula (II), eight of R¹, R², R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, R¹⁹, R²⁰, and R²¹ are deuterium. In another embodiment of Formula (II), nine of R¹, R², R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, R¹⁹, R²⁰, and R²¹ are deuterium. In another embodiment of Formula (II), ten of R¹, R², R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, R¹⁹, R²⁰, and R²¹ are deuterium. In another embodiment of Formula (II), eleven of R¹, R², R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, R¹⁹, R²⁰, and R²¹ are deuterium. In another embodiment of Formula (II), twelve of R¹, R², R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, R¹⁹, R²⁰, and R²¹ are deuterium. In another embodiment of Formula (II), thirteen of R¹, R², R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, R¹⁹, R²⁰, and R²¹ are deuterium. In another embodiment of Formula (II), fourteen of R¹, R², R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, R¹⁹, R²⁰, and R²¹ are deuterium. In another embodiment of Formula (II), fifteen of R¹, R², R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, R¹⁹, R²⁰, and R²¹ are deuterium. In another embodiment of Formula (II), sixteen of R¹, R², R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, R¹⁹, R²⁰, and R²¹ are deuterium. In another embodiment of Formula (II), seventeen of R¹, R², R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, R¹⁹, R²⁰, and R²¹ are deuterium. In another embodiment of Formula (II), eighteen of R¹, R², R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, R¹⁹, R²⁰, and R²¹ are deuterium. In another embodiment of Formula (II), nineteen of R¹, R², R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, R¹⁹, R²⁰, and R²¹ are deuterium. In another embodiment of Formula (II), twenty of R¹, R², R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, R¹⁹, R²⁰, and R²¹ are deuterium. In another embodiment of Formula (II), twenty-one of R¹, R², R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, R¹⁹, R²⁰, and R²¹ are deuterium. In another embodiment of Formula (II), twenty-two of R¹, R², R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, R¹⁹, R²⁰, and R²¹ are deuterium. In another embodiment of Formula (II), twenty-three of R¹, R², R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, R¹⁹, R²⁰, and R²¹ are deuterium. In another embodiment of Formula (II), twenty-four of R¹, R², R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, R¹⁹, R²⁰, and R²¹ are deuterium.

In one embodiment of Formula (II), R¹ and R² are each independently hydrogen, deuterium, or fluorine. In another embodiment of Formula (II), one of R¹ and R² is deuterium, and the other is hydrogen or fluorine. In another embodiment of Formula (II), R¹ and R² are each hydrogen, provided that at least one of R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, R¹⁹, R²⁰, and R²¹ is deuterium. In another embodiment of Formula (II), R¹ and R² are each deuterium. In another embodiment of Formula (II), R¹ and R² are each fluorine, provided that at least one of R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, R¹⁹, R²⁰, and R²¹ is deuterium.

In one embodiment of Formula (II), R³, R⁴, and R⁵, are each independently hydrogen or deuterium. In another embodiment of Formula (II), R³, R⁴, and R⁵, are each hydrogen provided that at least one of R¹, R², R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, R¹⁹, R²⁰, and R²¹ is deuterium. In another embodiment of Formula (II), one of R³, R⁴, and R⁵, is independently deuterium. In another embodiment of Formula (II), two of R³, R⁴, and R⁵ are deuterium. In another embodiment of Formula (II), R³, R⁴, and R⁵ are each deuterium.

In one embodiment of Formula (II), R⁶, R⁷, R⁸, and R⁹, are each independently hydrogen or deuterium. In another embodiment of Formula (II), R⁶, R⁷, R⁸, and R⁹, are each hydrogen provided that at least one of R¹, R², R³, R⁴, R⁵, R¹⁰, R¹¹, R¹², R¹³, R¹⁴, R¹⁵, R¹⁶, R^{17A}, R^{17B}, R^{17C}, R^{17D}, R¹⁸, R¹⁹, R²⁰, and R²¹ is deuterium. In one embodiment of Formula (II), one of R⁶, R⁷, R⁸, and R⁹, is deuterium. In another embodiment of Formula (II), two of R⁶, R⁷, R⁸, and R⁹, are deuterium. In another embodiment of Formula (II), three of R⁶, R⁷, R⁸, and R⁹, are deuterium. In another embodiment of Formula (II), R⁶, R⁷, R⁸, and R⁹ are each deuterium.

In one embodiment of Formula (II), R¹⁰, R¹⁴, R¹⁵, and R¹⁶, are each independently hydrogen or deuterium. In another embodiment of Formula (II), R¹⁰, R¹⁴, R¹⁵, and R¹⁶, are each hydrogen provided that at least one of R¹, R², R³, R⁴, R⁵, R⁶, R⁷, R⁸, R⁹, R¹¹, R¹², R¹³, R^{17A}, R^{17B}, R^{17C}, R^{17D}, R¹⁸, R¹⁹, R²⁰, and R²¹ is deuterium. In one embodiment of Formula (II), one of R¹⁰, R¹⁴, R¹⁵, and R¹⁶, is deuterium. In another embodiment of Formula (II), two of R¹⁰, R¹⁴, R¹⁵, and R¹⁶, are deuterium. In another embodiment of Formula (II), three of R¹⁰, R¹⁴, R¹⁵, and R¹⁶, are deuterium. In another embodiment of Formula (II), R¹⁰, R¹⁴, R¹⁵, and R¹⁶, are deuterium.

In one embodiment of Formula (II), R¹¹, R¹², and R¹³ are each independently hydrogen or deuterium. In another embodiment of Formula (II), R¹¹, R¹², and R¹³ are each hydrogen provided that at least one of R¹, R², R³, R⁴, R⁵, R⁶, R⁷, R⁸, R⁹, R¹⁰, R¹⁴, R¹⁵, R¹⁶, R^{17A}, R^{17B}, R^{17C}, R^{17D}, R¹⁸, R¹⁹, R²⁰, and R²¹ is deuterium. In one embodiment of Formula (II), one of R¹¹, R¹², and R¹³ is deuterium. In another embodiment of Formula (II), two of R¹¹, R¹², and R¹³ are deuterium. In another embodiment of Formula (II), R¹¹, R¹², and R¹³ are deuterium.

In one embodiment of Formula (II), R¹⁹, R²⁰, and R²¹ are each independently hydrogen, deuterium, or fluorine. In another embodiment of Formula (II), R¹⁹, R²⁰, and R²¹ are each hydrogen provided that at least one of R¹, R², R³, R⁴, R⁵, R⁶, R⁷, R⁸, R⁹, R¹⁰, R¹¹, R¹², R¹³, R¹⁴, R¹⁵, R¹⁶, R^{17A}, R^{17B}R^{17C}, R^{17D}, and R¹⁸ is deuterium. In another embodiment of Formula (II), R¹⁹, R²⁰, and R²¹ are each fluorine provided that at least one of R¹, R², R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, and R¹⁸ is deuterium. In another embodiment of Formula (II), one of R¹⁹, R²⁰, and R²¹ is fluorine provided that at least one of R¹, R², R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, and R¹⁸ is deuterium. In another embodiment of Formula (II), two of R¹⁹, R²⁰, and R²¹ are fluorine provided that at least one of R¹, R², R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, and R¹⁸ is deuterium. In another embodiment of Formula (II), one of R¹⁹, R²⁰, and R²¹ is deuterium or fluorine. In another embodiment of Formula (II), two of R¹⁹, R²⁰, and R²¹ are deuterium or fluorine. In another embodiment of Formula (II), R¹⁹, R²⁰, and R²¹ are deuterium.

In one embodiment of Formula (II), R¹⁸ is hydrogen or deuterium. In another embodiment of Formula (II), R¹⁸ is hydrogen provided that at least one of R¹, R², R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁹, R²⁰, and R²¹ is deuterium. In another embodiment of Formula (II), R¹⁸ is deuterium.

In one embodiment of Formula (II), R^{17A}, R^{17B}, R^{17C}, and R^{17D} are each independently hydrogen or deuterium. In another embodiment of Formula (II), R^{17A}, R^{17B}, R^{17C}, and R^{17D} are each hydrogen provided that at least one of R¹, R², R³, R⁴, R⁵, R⁶, R⁷, R⁸, R⁹, R¹⁰, R¹¹, R¹², R¹³, R¹⁴, R¹⁵, R¹⁶, R¹⁸, R¹⁹, R²⁰, and R²¹ is deuterium. In another embodiment of Formula (II), R^{17A} is deuterium; and R^{17B}, R^{17C}, and R^{17D} are each hydrogen. In another embodiment of Formula (II), R^{17B} is deuterium; and R^{17A}, R^{17C}, and R^{17D} are each hydrogen. In another embodiment of Formula (II), R^{17A} and R^{17C} are each deuterium; and R^{7B} and R^{17D} are each hydrogen. In another embodiment of Formula (II), R^{17B} and R^{17D} are each deuterium; and R^{17A} and R^{17C} are each hydrogen. In another embodiment of Formula (II), R^{17A} and R^{17B} are each deuterium; and R^{17C} and R^{17D} are each hydrogen. In another embodiment of Formula (II), R^{17A} and R^{17D} are each deuterium; and R^{17B} and R^{17C} are each hydrogen. In another embodiment of Formula (II), R^{17A}, R^{17B}, and R^{17C} are each deuterium; and R^{17D} is hydrogen. In another embodiment of Formula (II), R^{17A}, R^{17C}, and R^{17D} are each deuterium; and R^{17B} is hydrogen. In another embodiment of Formula (II), R^{17A}, R^{17B}, R^{17C}, and R^{17D} are each deuterium.

One embodiment pertains to compounds of Formula (II), or pharmaceutically acceptable salts thereof, wherein
R¹ and R² are each independently fluorine;
R³, R⁴, R⁵, R⁶, R⁷, R⁸, R⁹, R¹³, R¹⁴, R^{17B}, R^{17D}, R¹⁸, and R²⁰ are each independently hydrogen;
R¹⁰, R¹¹, R¹², R¹⁵, R¹⁶, R^{17A}, and R^{17C} are each independently deuterium; and
R¹⁹ and R²¹ are each independently fluorine.

One embodiment pertains to compounds of Formula (II), or pharmaceutically acceptable salts thereof, wherein
R¹ and R² are each independently fluorine;
R³, R⁴, R⁵, R¹⁰, R¹¹, R¹², R¹³, R¹⁴, R¹⁵, R¹⁶, R¹⁸, and R²⁰ are each independently hydrogen;
R⁶, R⁷, R⁸, and R⁹ are each independently deuterium;
R^{17A}, R^{17B}, R^{17C}, R^{17D} are each independently hydrogen; and
R¹⁹ and R²¹ are each independently fluorine.

### Formula (III)

One embodiment pertains to compounds of Formula (III), or pharmaceutically acceptable salts thereof,

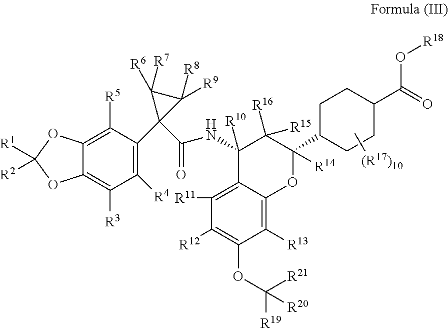

wherein
R¹ and R² are each independently hydrogen, deuterium, or fluorine;
R³, R⁴, R⁵, R⁶, R⁷, R⁸, R⁹, R¹⁰, R¹¹, R¹², R¹³, R¹⁴, R¹⁵, R¹⁶, and R¹⁸ are each independently hydrogen or deuterium;
R¹⁷, at each occurrence, is independently hydrogen or deuterium; and
R¹⁹, R²⁰, and R²¹ are each independently hydrogen, deuterium, or fluorine;
provided that, at least one of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ is deuterium.

In one embodiment of Formula (III), one of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ is deuterium. In another embodiment of Formula (III), two of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ are deuterium. In another embodiment of Formula (III), three of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ are deuterium. In another embodiment of Formula (III), four of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ are deuterium. In another embodiment of Formula (III), five of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ are deuterium. In another embodiment of Formula (III), six of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ are deuterium. In another embodiment of Formula (III), seven of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ are deuterium. In another embodiment of Formula (III), eight of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ are deuterium. In another embodiment of Formula (III), nine of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ are deuterium. In another embodiment of Formula (III), ten of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ are deuterium. In another embodiment of Formula (III), eleven of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ are deuterium. In another embodiment of Formula (III), twelve of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ are deuterium. In another embodiment of Formula (III), thirteen of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ are deuterium. In another embodiment of Formula (III), fourteen of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ are deuterium. In another embodiment of Formula (III), fifteen of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ are deuterium. In another embodiment of Formula (III), sixteen of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ are deuterium. In another embodiment of Formula (III), seventeen of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ are deuterium. In another embodiment of Formula (III), eighteen of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ are deuterium. In another embodiment of Formula (III), nineteen of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ are deuterium. In another embodiment of Formula (III), twenty of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ are deuterium. In another embodiment of Formula (III), twenty-one of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ are deuterium. In another embodiment of Formula (III), twenty-two of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ are deuterium. In another embodiment of Formula (III), twenty-three of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ are deuterium. In another embodiment of Formula (III), twenty-four of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ are deuterium. In another embodiment of Formula (III), twenty-five of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ are deuterium. In another embodiment of Formula (III), twenty-six of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ are deuterium. In another embodiment of Formula (III), twenty-seven of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ are deuterium. In another embodiment of Formula (III), twenty-eight of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ are deuterium. In another embodiment of Formula (III), twenty-nine of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ are deuterium. In another embodiment of Formula (III), thirty of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ are deuterium.

In one embodiment of Formula (III), R¹ and R² are each independently hydrogen, deuterium, or fluorine. In another embodiment of Formula (III), one of R¹ and R² is deuterium, and the other is hydrogen or fluorine. In another embodiment of Formula (III), R¹ and R² are each hydrogen, provided that at least one of R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ is deuterium. In another embodiment of Formula (III), R¹ and R² are each deuterium. In another embodiment of Formula (III), R¹ and R² are each fluorine, provided that at least one of R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ is deuterium.

In one embodiment of Formula (III), R³, R⁴, and R⁵, are each independently hydrogen or deuterium. In another embodiment of Formula (III), R³, R⁴, and R⁵, are each hydrogen provided that at least one of R¹, R², R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ is deuterium. In another embodiment of Formula (III), one of R³, R⁴, and R⁵, is independently deuterium. In another embodiment of Formula (III), two of R³, R⁴, and R⁵, are deuterium. In another embodiment of Formula (III), R³, R⁴, and R⁵ are each deuterium.

In one embodiment of Formula (III), R⁶, R⁷, R⁸, and R⁹, are each independently hydrogen or deuterium. In another embodiment of Formula (III), R⁶, R⁷, R⁸, and R⁹, are each hydrogen provided that at least one of R¹, R², R³, R⁴, R⁵, R¹⁰, R¹¹, R¹², R¹³, R¹⁴, R¹⁵, R¹⁶, R¹⁷, R¹⁸, R¹⁹, R²⁰, and R²¹ is deuterium. In one embodiment of Formula (III), one of R⁶, R⁷, R⁸, and R⁹, is deuterium. In another embodiment of Formula (III), two of R⁶, R⁷, R⁸, and R⁹, are deuterium. In another embodiment of Formula (III), three of R⁶, R⁷, R⁸, and R⁹, are deuterium. In another embodiment of Formula (III), R⁶, R⁷, R⁸, and R⁹ are each deuterium.

In one embodiment of Formula (III), R¹⁰, R¹⁴, R¹⁵, and R¹⁶, are each independently hydrogen or deuterium. In another embodiment of Formula (III), R¹⁰, R¹⁴, R¹⁵, and R¹⁶, are each hydrogen provided that at least one of R¹, R², R³, R⁴, R⁵, R⁶, R⁷, R⁸, R⁹, R¹¹, R¹², R¹³, R¹⁷, R¹⁸, R¹⁹, R²⁰, and R²¹ is deuterium. In one embodiment of Formula (III), one of R¹⁰, R¹⁴, R¹⁵, and R¹⁶, is deuterium. In another embodiment of Formula (III), two of R¹⁰, R¹⁴, R¹⁵, and R¹⁶, are deuterium. In another embodiment of Formula (III), three of R¹⁰, R¹⁴, R¹⁵, and R¹⁶, are deuterium. In another embodiment of Formula (III), R¹⁰, R¹⁴, R¹⁵, and R¹⁶, are deuterium.

In one embodiment of Formula (III), R¹¹, R¹², and R¹³ are each independently hydrogen or deuterium. In another embodiment of Formula (III), R¹¹, R¹², and R¹³ are each hydrogen provided that at least one of R¹, R², R³, R⁴, R⁵, R⁶, R⁷, R⁸, R⁹, R¹⁰, R¹⁴, R¹⁵, R¹⁶, R¹⁷, R¹⁸, R¹⁹, R²⁰, and R²¹ is deuterium. In one embodiment of Formula (III), one of R¹¹, R¹², and R¹³ is deuterium. In another embodiment of Formula (III), two of R¹¹, R¹², and R¹³ are deuterium. In another embodiment of Formula (III), R¹¹, R¹², and R¹³ are deuterium.

In one embodiment of Formula (III), R¹⁹, R²⁰, and R²¹ are each independently hydrogen, deuterium, or fluorine. In another embodiment of Formula (III), R¹⁹, R²⁰, and R²¹ are each hydrogen provided that at least one of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, and R¹⁸ is deuterium. In another embodiment of Formula (III), R¹⁹, R²⁰, and R²¹ are each fluorine provided that at least one of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, and R¹⁸ is deuterium. In another embodiment of Formula (III), one of R¹⁹, R²⁰, and R²¹ is fluorine provided that at least one of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, and R¹⁸ is deuterium. In another embodiment of Formula (III), two of R¹⁹, R²⁰, and R²¹ are fluorine provided that at least one of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, and R¹⁸ is deuterium. In another embodiment of Formula (III), one of R¹⁹, R²⁰, and R²¹ is deuterium or fluorine. In another embodiment of Formula (III), two of R¹⁹, R²⁰, and R²¹ are deuterium or fluorine. In another embodiment of Formula (III), R¹⁹, R²⁰, and R²¹ are deuterium.

In one embodiment of Formula (III), R¹⁸ is hydrogen or deuterium. In another embodiment of Formula (III), R¹⁸ is hydrogen provided that at least one of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁹, R²⁰, and R²¹ is deuterium. In another embodiment of Formula (III), R¹⁸ is deuterium.

In one embodiment of Formula (III), each R¹⁷ is independently hydrogen or deuterium. In another embodiment of Formula (III), each R¹⁷ is independently hydrogen provided that at least one of R¹, R², R³, R⁴, R⁵, R⁶, R⁷, R⁸, R⁹, R¹⁰, R¹¹, R¹², R¹³, R¹⁴, R¹⁵, R¹⁶, R¹⁸, R¹⁹, R²⁰, and R²¹ is deuterium. In another embodiment of Formula (III), one R¹⁷ is deuterium. In another embodiment of Formula (III), two R¹⁷ are deuterium. In another embodiment of Formula (III), three R¹⁷ are deuterium. In another embodiment of Formula (III), four R¹⁷ are deuterium. In another embodiment of Formula (III), five R¹⁷ are deuterium. In another embodiment of Formula (III), six R¹⁷ are deuterium. In another embodiment of Formula (III), seven R¹⁷ are deuterium. In another embodiment of Formula (III), eight R¹⁷ are deuterium. In another embodiment of Formula (III), nine R¹⁷ are deuterium. In another embodiment of Formula (III), ten R¹⁷ are deuterium.

One embodiment pertains to compounds of Formula (IIIa),

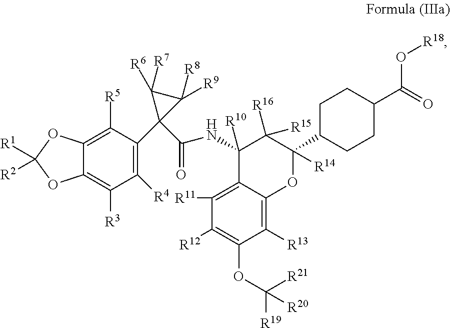

wherein R¹, R², R³, R⁴, R⁵, R⁶, R⁷, R⁸, R⁹, R¹⁰, R¹¹, R¹², R¹³, R¹⁴, R¹⁵, R¹⁶, R¹⁸, R¹⁹, R²⁰, and R²¹ are as described in embodiments of Formula (III) herein.

One embodiment pertains to compounds of Formula (IIIb),

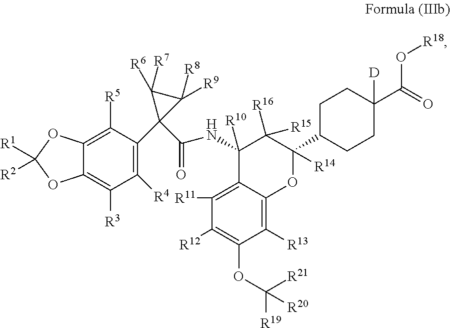

wherein R¹, R², R³, R⁴, R⁵, R⁶, R⁷, R⁸, R⁹, R¹⁰, R¹¹, R¹², R¹³, R¹⁴, R¹⁵, R¹⁶, R¹⁸, R¹⁹, R²⁰, and R²¹ are as described in embodiments of Formula (III) herein.

One embodiment pertains to compounds of Formula (IIIc),

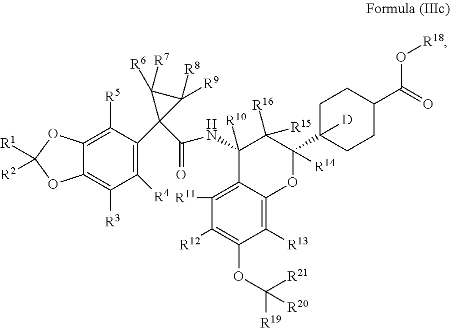

wherein R¹, R², R³, R⁴, R⁵, R⁶, R⁷, R⁸, R⁹, R¹⁰, R¹¹, R¹², R¹³, R¹⁴, R¹⁵, R¹⁶, R¹⁸, R¹⁹, R²⁰, and R²¹ are as described in embodiments of Formula (III) herein.

One embodiment pertains to compounds of Formula (IIId),

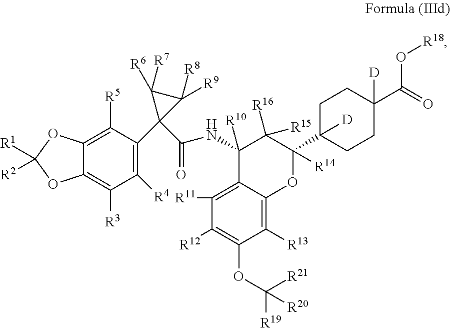

wherein R¹, R², R³, R⁴, R⁵, R⁶, R⁷, R⁸, R⁹, R¹⁰, R¹¹, R¹², R¹³, R¹⁴, R¹⁵, R¹⁶, R¹⁸, R¹⁹, R²⁰, and R²¹ are as described in embodiments of Formula (III) herein.

One embodiment pertains to compounds of Formula (IIIe),

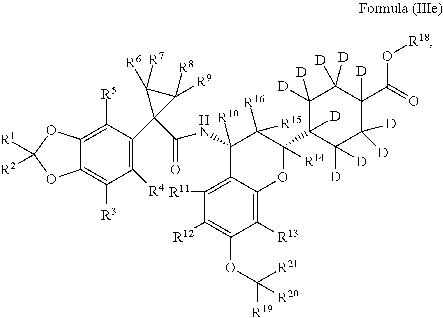

wherein R¹, R², R³, R⁴, R⁵, R⁶, R⁷, R⁸, R⁹, R¹⁰, R¹¹, R¹², R¹³, R¹⁴, R¹⁵, R¹⁶, R¹⁸, R¹⁹, R²⁰, and R²¹ are as described in embodiments of Formula (III) herein.

### Formula (IV)

One embodiment pertains to compounds of Formula (IV), or pharmaceutically acceptable salts thereof,

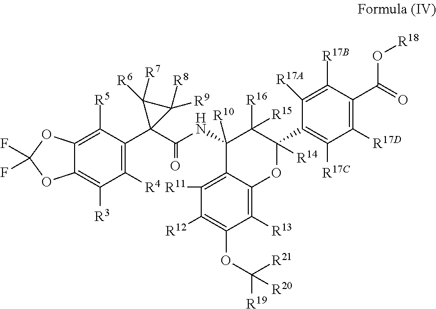

wherein
R³, R⁴, R⁵, R⁶, R⁷, R⁸, R⁹, R¹⁰, R¹¹, R¹², R¹³, R¹⁴, R¹⁵, R¹⁶, and R¹⁸ are each independently hydrogen or deuterium;
R^{17A}, R^{17B}, R^{17C}, R^{17D} are each independently hydrogen or deuterium; and
R¹⁹, R²⁰, and R²¹ are each independently hydrogen, deuterium, or fluorine;
provided that, at least one of R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, R¹⁹, R²⁰, and R²¹ is deuterium.

In one embodiment of Formula (IV), one of R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, R¹⁹, R²⁰, and R²¹ is deuterium. In another embodiment of Formula (IV), two of R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, R¹⁹, R²⁰, and R²¹ are deuterium. In another embodiment of Formula (IV), three of R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, R¹⁹, R²⁰, and R²¹ are deuterium. In another embodiment of Formula (IV), four of R³, R⁴, R⁵, R⁶, R⁷, R⁸, R⁹, R¹⁰, R¹¹, R¹², R¹³, R¹⁴, R¹⁵, R¹⁶, R^{17A}, R^{17B}R^{17C}, R^{17D}, R¹⁸, R¹⁹, R²⁰, and R²¹ are deuterium. In another embodiment of Formula (IV), five of R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, R¹⁹, R²⁰, and R²¹ are deuterium. In another embodiment of Formula (IV), six of R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, R¹⁹, R²⁰, and R²¹ are deuterium. In another embodiment of Formula (IV), seven of R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, R¹⁹, R²⁰, and R²¹ are deuterium. In another embodiment of Formula (IV), eight of R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, R¹⁹, R²⁰, and R²¹ are deuterium. In another embodiment of Formula (IV), nine of R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, R¹⁹, R²⁰, and R²¹ are deuterium. In another embodiment of Formula (IV), ten of R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, R¹⁹, R²⁰, and R²¹ are deuterium. In another embodiment of Formula (IV), eleven of R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, R¹⁹, R²⁰, and R²¹ are deuterium. In another embodiment of Formula (IV), twelve of R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, R¹⁹, R²⁰, and R²¹ are deuterium. In another embodiment of Formula (IV), thirteen of R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, R¹⁹, R²⁰, and R²¹ are deuterium. In another embodiment of Formula (IV), fourteen of R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, R¹⁹, R²⁰, and R²¹ are deuterium. In another embodiment of Formula (IV), fifteen of R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, R¹⁹, R²⁰, and R²¹ are deuterium. In another embodiment of Formula (IV), sixteen of R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, R¹⁹, R²⁰, and R²¹ are deuterium. In another embodiment of Formula (IV), seventeen of R³, R⁴, R⁵, R⁶, R⁷, R⁸, R⁹, R¹⁰, R¹¹, R¹², R¹³, R¹⁴, R¹⁵, R¹⁶, R^{17A}R^{17B}, R^{17C}, R^{17D}, R¹⁸, R¹⁹, R²⁰, and R²¹ are deuterium. In another embodiment of Formula (IV), eighteen of R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, R¹⁹, R²⁰, and R²¹ are deuterium. In another embodiment of Formula (IV), nineteen of R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, R¹⁹, R²⁰, and R²¹ are deuterium. In another embodiment of Formula (IV), twenty of R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, R¹⁹, R²⁰, and R²¹ are deuterium. In another embodiment of Formula (IV), twenty-one of R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, R¹⁹, R²⁰, and R²¹ are deuterium. In another embodiment of Formula (IV), twenty-two of R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, R¹⁹, R²⁰, and R²¹ are deuterium.

In one embodiment of Formula (IV), R³, R⁴, and R⁵, are each independently hydrogen or deuterium. In another embodiment of Formula (IV), R³, R⁴, and R⁵, are each hydrogen provided that at least one of R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, R¹⁹, R²⁰, and R²¹ is deuterium. In another embodiment of Formula (IV), one of R³, R⁴, and R⁵, is independently deuterium. In another embodiment of Formula (IV), two of R³, R⁴, and R⁵, are deuterium. In another embodiment of Formula (IV), R³, R⁴, and R⁵ are each deuterium.

In one embodiment of Formula (IV), R⁶, R⁷, R⁸, and R⁹, are each independently hydrogen or deuterium. In another embodiment of Formula (IV), R⁶, R⁷, R⁸, and R⁹, are each hydrogen provided that at least one of R³, R⁴, R⁵, R¹⁰, R¹¹, R¹², R¹³, R¹⁴, R¹⁵, R¹⁶, R^{17A}, R^{17B}, R^{17C}, R^{17D}, R¹⁸, R¹⁹, R²⁰, and R²¹ is deuterium. In one embodiment of Formula (IV), one of R⁶, R⁷, R⁸, and R⁹, is deuterium. In another embodiment of Formula (IV), two of R⁶, R⁷, R⁸, and R⁹, are deuterium. In another embodiment of Formula (IV), three of R⁶, R⁷, R⁸, and R⁹, are deuterium. In another embodiment of Formula (IV), R⁶, R⁷, R⁸, and R⁹ are each deuterium.

In one embodiment of Formula (IV), R¹⁰, R¹⁴, R¹⁵, and R¹⁶, are each independently hydrogen or deuterium. In another embodiment of Formula (IV), R¹⁰, R¹⁴, R¹⁵, and R¹⁶, are each hydrogen provided that at least one of R³, R⁴, R⁵, R⁶, R⁷, R⁸, R⁹, R¹¹, R¹², R¹³, R^{17A}, R^{17B}, R^{17C}, R^{17D}, R¹⁸, R¹⁹, R²⁰, and R²¹ is deuterium. In one embodiment of Formula (IV), one of R¹⁰, R¹⁴, R¹⁵, and R¹⁶, is deuterium. In another embodiment of Formula (IV), two of R¹⁰, R¹⁴, R¹⁵, and R¹⁶, are deuterium. In another embodiment of Formula (IV), three of R¹⁰, R¹⁴, R¹⁵, and R¹⁶, are deuterium. In another embodiment of Formula (IV), R¹⁰, R¹⁴, R¹⁵, and R¹⁶, are deuterium.

In one embodiment of Formula (IV), R¹¹, R¹², and R¹³ are each independently hydrogen or deuterium. In another embodiment of Formula (IV), R¹¹, R¹², and R¹³ are each hydrogen provided that at least one of R³, R⁴, R⁵, R⁶, R⁷, R⁸, R⁹, R¹⁰, R¹⁴, R¹⁵, R¹⁶, R^{17A}, R^{17B}, R^{17C}, R^{17D}, R¹⁸, R¹⁹, R²⁰, and R²¹ is deuterium. In one embodiment of Formula (IV), one of R¹¹, R¹², and R¹³ is deuterium. In another embodiment of Formula (IV), two of R¹¹, R¹², and R¹³ are deuterium. In another embodiment of Formula (IV), R¹¹, R¹², and R¹³ are deuterium.

In one embodiment of Formula (IV), R¹⁹, R²⁰, and R²¹ are each independently hydrogen, deuterium, or fluorine. In another embodiment of Formula (IV), R¹⁹, R²⁰, and R²¹ are each hydrogen provided that at least one of R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, and R¹⁸ is deuterium. In another embodiment of Formula (IV), R¹⁹, R²⁰, and R²¹ are each fluorine provided that at least one of R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, and R¹⁸ is deuterium. In another embodiment of Formula (IV), one of R¹⁹, R²⁰, and R²¹ is fluorine provided that at least one of R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, and R¹⁸ is deuterium. In another embodiment of Formula (IV), two of R¹⁹, R²⁰, and R²¹ are fluorine provided that at least one of R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, and R¹⁸ is deuterium. In another embodiment of Formula (IV), one of R¹⁹, R²⁰, and R²¹ is deuterium or fluorine. In another embodiment of Formula (IV), two of R¹⁹, R²⁰, and R²¹ are deuterium or fluorine. In another embodiment of Formula (IV), R¹⁹, R²⁰, and R²¹ are deuterium.

In one embodiment of Formula (IV), R¹⁸ is hydrogen or deuterium. In another embodiment of Formula (IV), R¹⁸ is hydrogen provided that at least one of R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁹, R²⁰, and R²¹ is deuterium. In another embodiment of Formula (IV), R¹⁸ is deuterium.

In one embodiment of Formula (IV), R^{17A}, R^{17B}, R^{17C}, and R^{17D} are each independently hydrogen or deuterium. In another embodiment of Formula (IV), R^{17A}, R^{17B}, R^{17C}, and R^{17D} are each hydrogen provided that at least one of R³, R⁴, R⁵, R⁶, R⁷, R⁸, R⁹, R¹⁰, R¹¹, R¹², R¹³, R¹⁴, R¹⁵, R¹⁶, R¹⁸, R¹⁹, R²⁰, and R²¹ is deuterium. In another embodiment of Formula (IV), R^{17A} is deuterium; and R^{17B}, R^{17C}, and R^{17D} are each hydrogen. In another embodiment of Formula (IV), R^{17B} is deuterium; and R^{17A}, R^{17C}, and R^{17D} are each hydrogen. In another embodiment of Formula (IV), R^{17A} and R^{17C} are deuterium; and R^{17B} and R^{17D} are each hydrogen. In another embodiment of Formula (IV), R^{17B} and R^{17D} are deuterium; and R^{17A} and R^{17C} are each hydrogen. In another embodiment of Formula (IV), R^{17A} and R^{17B} are deuterium; and R^{17C} and R^{17D} are each hydrogen. In another embodiment of Formula (IV), R^{17A} and R^{17D} are deuterium; and R^{17B} and R^{17C} are each hydrogen. In another embodiment of Formula (IV), R^{17A}, R^{17B} and R^{17C} are deuterium; and R^{17D} is hydrogen. In another embodiment of Formula (IV), R^{17A}, R^{17C}, and R^{17D} are deuterium; and R^{17B} is hydrogen. In another embodiment of Formula (IV), R^{17A}, R^{17B}, R^{17C}, and R^{17D} are each deuterium.

One embodiment pertains to compounds of Formula (IV), or pharmaceutically acceptable salts thereof, wherein
R³, R⁴, R⁵, R⁶, R⁷, R⁸, R⁹, R¹³, R¹⁴, R^{17B}, R^{17D}, R¹⁸, and R²⁰ are each independently hydrogen;
R¹⁰, R¹¹, R¹², R¹⁵, R¹⁶, R^{17A}, and R^{17C} are each independently deuterium; and
R¹⁹ and R²¹ are each independently fluorine.

One embodiment pertains to compounds of Formula (IV), or pharmaceutically acceptable salts thereof, wherein
R³, R⁴, R⁵, R¹⁰, R¹¹, R¹², R¹³, R¹⁴, R¹⁵, R¹⁶, R¹⁸, and R²⁰ are each independently hydrogen;
R⁶, R⁷, R⁸, and R⁹ are each independently deuterium;
R^{17A}, R^{17B}, R^{17C}, R^{17D} are each independently hydrogen; and
R¹⁹ and R²¹ are each independently fluorine.

### Formula (V)

One embodiment pertains to compounds of Formula (V), or pharmaceutically acceptable salts thereof,

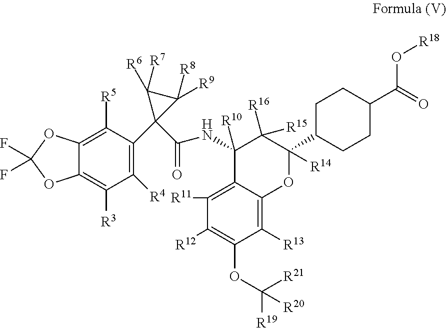

wherein
R³, R⁴, R⁵, R⁶, R⁷, R⁸, R⁹, R¹⁰, R¹¹, R¹², R¹³, R¹⁴, R¹⁵, R¹⁶, and R¹⁸ are each independently hydrogen or deuterium; and
R¹⁹, R²⁰, and R²¹ are each independently hydrogen, deuterium, or fluorine;
provided that, at least one of R³, R⁴, R⁵, R⁶, R⁷, R⁸, R⁹, R¹⁰, R¹¹, R¹², R¹³, R¹⁴, R¹⁵, R¹⁶, R¹⁸, R¹⁹, R²⁰, and R²¹ is deuterium.

In one embodiment of Formula (V), one of R³, R⁴, R⁵, R⁶, R⁷, R⁸, R⁹, R¹⁰, R¹¹, R¹², R¹³, R¹⁴, R¹⁵, R¹⁶, R¹⁸, R¹⁹, R²⁰, and R²¹ is deuterium. In another embodiment of Formula (V), two of R³, R⁴, R⁵, R⁶, R⁷, R⁸, R⁹, R¹⁰, R¹¹, R¹², R¹³, R¹⁴, R¹⁵, R¹⁶, R¹⁸, R¹⁹, R²⁰, and R²¹ are deuterium. In another embodiment of Formula (V), three of R³, R⁴, R⁵, R⁶, R⁷, R⁸, R⁹, R¹⁰, R¹¹, R¹², R¹³, R¹⁴, R¹⁵, R¹⁶, R¹⁸, R¹⁹, R²⁰, and R²¹ are deuterium. In another embodiment of Formula (V), four of R³, R⁴, R⁵, R⁶, R⁷, R⁸, R⁹, R¹⁰, R¹¹, R¹², R¹³, R¹⁴, R¹⁵, R¹⁶, R¹⁸, R¹⁹, R²⁰, and R²¹ are deuterium. In another embodiment of Formula (V), five of R³, R⁴, R⁵, R⁶, R⁷, R⁸, R⁹, R¹⁰, R¹¹, R¹², R¹³, R¹⁴, R¹⁵, R¹⁶, R¹⁸, R¹⁹, R²⁰, and R²¹ are deuterium. In another embodiment of Formula (V), six of R³, R⁴, R⁵, R⁶, R⁷, R⁸, R⁹, R¹⁰, R¹¹, R¹², R¹³, R¹⁴, R¹⁵, R¹⁶, R¹⁸, R¹⁹, R²⁰, and R²¹ are deuterium. In another embodiment of Formula (V), seven of R³, R⁴, R⁵, R⁶, R⁷, R⁸, R⁹, R¹⁰, R¹¹, R¹², R¹³, R¹⁴, R¹⁵, R¹⁶, R¹⁸, R¹⁹, R²⁰, and R²¹ are deuterium. In another embodiment of Formula (V), eight of R³, R⁴, R⁵, R⁶, R⁷, R⁸, R⁹, R¹⁰, R¹¹, R¹², R¹³, R¹⁴, R¹⁵, R¹⁶, R¹⁸, R¹⁹, R²⁰, and R²¹ are deuterium. In another embodiment of Formula (V), nine of R³, R⁴, R⁵, R⁶, R⁷, R⁸, R⁹, R¹⁰, R¹¹, R¹², R¹³, R¹⁴, R¹⁵, R¹⁶, R¹⁸, R¹⁹, R²⁰, and R²¹ are deuterium. In another embodiment of Formula (V), ten of R³, R⁴, R⁵, R⁶, R⁷, R⁸, R⁹, R¹⁰, R¹¹, R¹², R¹³, R¹⁴, R¹⁵, R¹⁶, R¹⁸, R¹⁹, R²⁰, and R²¹ are deuterium. In another embodiment of Formula (V), eleven of R³, R⁴, R⁵, R⁶, R⁷, R⁸, R⁹, R¹⁰, R¹¹, R¹², R¹³, R¹⁴, R¹⁵, R¹⁶, R¹⁸, R¹⁹, R²⁰, and R²¹ are deuterium. In another embodiment of Formula (V), twelve of R³, R⁴, R⁵, R⁶, R⁷, R⁸, R⁹, R¹⁰, R¹¹, R¹², R¹³, R¹⁴, R¹⁵, R¹⁶, R¹⁸, R¹⁹, R²⁰, and R²¹ are deuterium. In another embodiment of Formula (V), thirteen of R³, R⁴, R⁵, R⁶, R⁷, R⁸, R⁹, R¹⁰, R¹¹, R¹², R¹³, R¹⁴, R¹⁵, R¹⁶, R¹⁸, R¹⁹, R²⁰, and R²¹ are deuterium. In another embodiment of Formula (V), fourteen of R³, R⁴, R⁵, R⁶, R⁷, R⁸, R⁹, R¹⁰, R¹¹, R¹², R¹³, R¹⁴, R¹⁵, R¹⁶, R¹⁸, R¹⁹, R²⁰, and R²¹ are deuterium. In another embodiment of Formula (V), fifteen of R³, R⁴, R⁵, R⁶, R⁷, R⁸, R⁹, R¹⁰, R¹¹, R¹², R¹³, R¹⁴, R¹⁵, R¹⁶, R¹⁸, R¹⁹, R²⁰, and R²¹ are deuterium. In another embodiment of Formula (V), sixteen of R³, R⁴, R⁵, R⁶, R⁷, R⁸, R⁹, R¹⁰, R¹¹, R¹², R¹³, R¹⁴, R¹⁵, R¹⁶, R¹⁸, R¹⁹, R²⁰, and R²¹ are deuterium. In another embodiment of Formula (V), seventeen of R³, R⁴, R⁵, R⁶, R⁷, R⁸, R⁹, R¹⁰, R¹¹, R¹², R¹³, R¹⁴, R¹⁵, R¹⁶, R¹⁸, R¹⁹, R²⁰, and R²¹ are deuterium. In another embodiment of Formula (V), eighteen of R³, R⁴, R⁵, R⁶, R⁷, R⁸, R⁹, R¹⁰, R¹¹, R¹², R¹³, R¹⁴, R¹⁵, R¹⁶, R¹⁸, R¹⁹, R²⁰, and R²¹ are deuterium.

In one embodiment of Formula (V), R³, R⁴, and R⁵, are each independently hydrogen or deuterium. In another embodiment of Formula (V), R³, R⁴, and R⁵, are each hydrogen provided that at least one of R⁶, R⁷, R⁸, R⁹, R¹⁰, R¹¹, R¹², R¹³, R¹⁴, R¹⁵, R¹⁶, R¹⁸, R¹⁹, R²⁰, and R²¹ is deuterium. In another embodiment of Formula (V), one of R³, R⁴, and R⁵, is independently deuterium. In another embodiment of Formula (V), two of R³, R⁴, and R⁵, are deuterium. In another embodiment of Formula (V), R³, R⁴, and R⁵ are each deuterium.

In one embodiment of Formula (V), R⁶, R⁷, R⁸, and R⁹, are each independently hydrogen or deuterium. In another embodiment of Formula (V), R⁶, R⁷, R⁸, and R⁹, are each hydrogen provided that at least one of R³, R⁴, R⁵, R¹⁰, R¹¹, R¹², R¹³, R¹⁴, R¹⁵, R¹⁶, R¹⁸, R¹⁹, R²⁰, and R²¹ is deuterium. In one embodiment of Formula (V), one of R⁶, R⁷, R⁸, and R⁹, is deuterium. In another embodiment of Formula (V), two of R⁶, R⁷, R⁸, and R⁹, are deuterium. In another embodiment of Formula (V), three of R⁶, R⁷, R⁸, and R⁹, are deuterium. In another embodiment of Formula (V), R⁶, R⁷, R⁸, and R⁹ are each deuterium.

In one embodiment of Formula (V), R¹⁰, R¹⁴, R¹⁵, and R¹⁶, are each independently hydrogen or deuterium. In another embodiment of Formula (V), R¹⁰, R¹⁴, R¹⁵, and R¹⁶, are each hydrogen provided that at least one of R³, R⁴, R⁵, R⁶, R⁷, R⁸, R⁹, R¹¹, R¹², R¹³, R¹⁸, R¹⁹, R²⁰, and R²¹ is deuterium. In one embodiment of Formula (V), one of R¹⁰, R¹⁴, R¹⁵, and R¹⁶, is deuterium. In another embodiment of Formula (V), two of R¹⁰, R¹⁴, R¹⁵, and R¹⁶, are deuterium. In another embodiment of Formula (V), three of R¹⁰, R¹⁴, R¹⁵, and R¹⁶, are deuterium. In another embodiment of Formula (V), R¹⁰, R¹⁴, R¹⁵, and R¹⁶, are deuterium.

In one embodiment of Formula (V), R¹¹, R¹², and R¹³ are each independently hydrogen or deuterium. In another embodiment of Formula (V), R¹¹, R¹², and R¹³ are each hydrogen provided that at least one of R³, R⁴, R⁵, R⁶, R⁷, R⁸, R⁹, R¹⁰, R¹⁴, R¹⁵, R¹⁶, R¹⁸, R¹⁹, R²⁰, and R²¹ is deuterium. In one embodiment of Formula (V), one of R¹¹, R¹², and R¹³ is deuterium. In another embodiment of Formula (V), two of R¹¹, R¹², and R¹³ are deuterium. In another embodiment of Formula (V), R¹¹, R¹², and R¹³ are deuterium.

In one embodiment of Formula (V), R¹⁹, R²⁰, and R²¹ are each independently hydrogen, deuterium, or fluorine. In another embodiment of Formula (V), R¹⁹, R²⁰, and R²¹ are each hydrogen provided that at least one of R³, R⁴, R⁵, R⁶, R⁷, R⁸, R⁹, R¹⁰, R¹¹, R¹², R¹³, R¹⁴, R¹⁵, R¹⁶, and R¹⁸ is deuterium. In another embodiment of Formula (V), R¹⁹, R²⁰, and R²¹ are each fluorine provided that at least one of R³, R⁴, R⁵, R⁶, R⁷, R⁸, R⁹, R¹⁰, R¹¹, R¹², R¹³, R¹⁴, R¹⁵, R¹⁶, and R¹⁸ is deuterium. In another embodiment of Formula (V), one of R¹⁹, R²⁰, and R²¹ is fluorine provided that at least one of R³, R⁴, R⁵, R⁶, R⁷, R⁸, R⁹, R¹⁰, R¹¹, R¹², R¹³, R¹⁴, R¹⁵, R¹⁶, and R¹⁸ is deuterium. In another embodiment of Formula (V), two of R¹⁹, R²⁰, and R²¹ are fluorine provided that at least one of R³, R⁴, R⁵, R⁶, R⁷, R⁸, R⁹, R¹⁰, R¹¹, R¹², R¹³, R¹⁴, R¹⁵, R¹⁶, and R¹⁸ is deuterium. In another embodiment of Formula (V), one of R¹⁹, R²⁰, and R²¹ is deuterium or fluorine. In another embodiment of Formula (V), two of R¹⁹, R²⁰, and R²¹ are deuterium or fluorine. In another embodiment of Formula (V), R¹⁹, R²⁰, and R²¹ are deuterium.

In one embodiment of Formula (V), R¹⁸ is hydrogen or deuterium. In another embodiment of Formula (V), R¹⁸ is hydrogen provided that at least one of R³, R⁴, R⁵, R⁶, R⁷, R⁸, R⁹, R¹⁰, R¹¹, R¹², R¹³, R¹⁴, R¹⁵, R¹⁶, R¹⁹, R²⁰, and R²¹ is deuterium. In another embodiment of Formula (V), R¹⁸ is deuterium.

One embodiment pertains to compounds of Formula (Va),

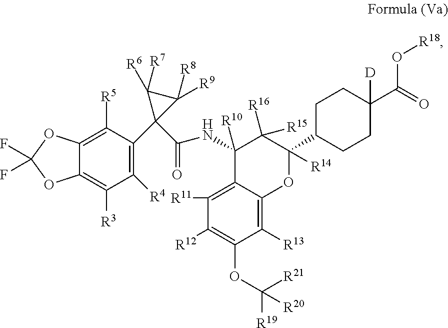

wherein R³, R⁴, R⁵, R⁶, R⁷, R⁸, R⁹, R¹⁰, R¹¹, R¹², R¹³, R¹⁴, R¹⁵, R¹⁶, R¹⁸, R¹⁹, R²⁰, and R²¹ are as described in embodiments of Formula (V) herein.

One embodiment pertains to compounds of Formula (Vb),

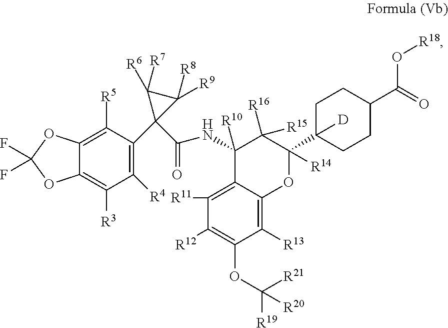

wherein R³, R⁴, R⁵, R⁶, R⁷, R⁸, R⁹, R¹⁰, R¹¹, R¹², R¹³, R¹⁴, R¹⁵, R¹⁶, R¹⁸, R¹⁹, R²⁰, and R²¹ are as described in embodiments of Formula (V) herein.

One embodiment pertains to compounds of Formula (Vc),

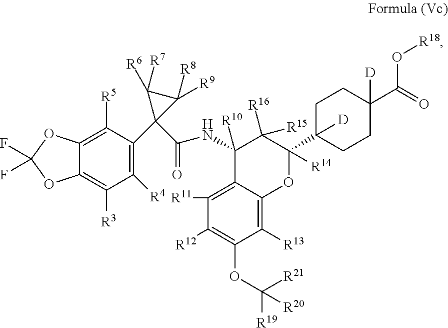

wherein R³, R⁴, R⁵, R⁶, R⁷, R⁸, R⁹, R¹⁰, R¹¹, R¹², R¹³, R¹⁴, R¹⁵, R¹⁶, R¹⁸, R¹⁹, R²⁰, and R²¹ are as described in embodiments of Formula (V) herein.

One embodiment pertains to compounds of Formula (Vd),

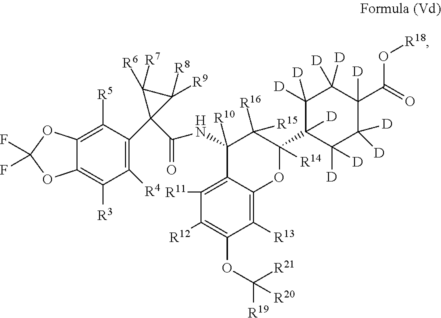

wherein R³, R⁴, R⁵, R⁶, R⁷, R⁸, R⁹, R¹⁰, R¹¹, R¹², R¹³, R¹⁴, R¹⁵, R¹⁶, R¹⁸, R¹⁹, R²⁰, and R²¹ are as described in embodiments of Formula (V) herein.

### Formula (VI)

One embodiment pertains to compounds of Formula (VI), or pharmaceutically acceptable salts thereof,

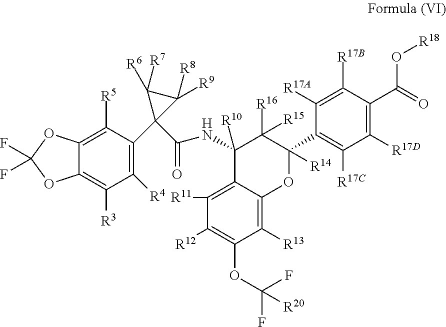

wherein
R³, R⁴, R⁵, R⁶, R⁷, R⁸, R⁹, R¹⁰, R¹¹, R¹², R¹³, R¹⁴, R¹⁵, R¹⁶, and R¹⁸ are each independently hydrogen or deuterium;
R^{17A}, R^{17B}, R^{17C}, R^{17D} are each independently hydrogen or deuterium; and
R²⁰ is independently hydrogen, deuterium, or fluorine;
provided that, at least one of R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, and R²⁰ is deuterium.

In one embodiment of Formula (VI), one of R³, R⁴, R⁵, R⁶, R⁷, R⁸, R⁹, R¹⁰, R¹¹, R¹², R¹³, R¹⁴, R¹⁵, R⁶, R^{17A}, R^{17B}, R^{17C}, R^{17D}, R¹⁸, and R²⁰ is deuterium. In another embodiment of Formula (VI), two of R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, and R²⁰ are deuterium. In another embodiment of Formula (VI), three of R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, and R²⁰ are deuterium. In another embodiment of Formula (VI), four of R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, and R²⁰ are deuterium. In another embodiment of Formula (VI), five of R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, and R²⁰ are deuterium. In another embodiment of Formula (VI), six of R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, and R²⁰ are deuterium. In another embodiment of Formula (VI), seven of R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, and R²⁰ are deuterium. In another embodiment of Formula (VI), eight of R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, and R²⁰ are deuterium. In another embodiment of Formula (VI), nine of R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, and R²⁰ are deuterium. In another embodiment of Formula (VI), ten of R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, and R²⁰ are deuterium. In another embodiment of Formula (VI), eleven of R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, and R²⁰ are deuterium. In another embodiment of Formula (VI), twelve of R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, and R²⁰ are deuterium. In another embodiment of Formula (VI), thirteen of R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, and R²⁰ are deuterium. In another embodiment of Formula (VI), fourteen of R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, and R²⁰ are deuterium. In another embodiment of Formula (VI), fifteen of R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, and R²⁰ are deuterium. In another embodiment of Formula (VI), sixteen of R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, and R²⁰ are deuterium. In another embodiment of Formula (VI), seventeen of R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, and R²⁰ are deuterium. In another embodiment of Formula (VI), eighteen of R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, and R²⁰ are deuterium. In another embodiment of Formula (VI), nineteen of R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, and R²⁰ are deuterium. In another embodiment of Formula (VI), twenty of R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, and R²⁰ are deuterium.

In one embodiment of Formula (VI), R³, R⁴, and R⁵, are each independently hydrogen or deuterium. In another embodiment of Formula (VI), R³, R⁴, and R⁵, are each hydrogen provided that at least one of R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, and R²⁰ is deuterium. In another embodiment of Formula (VI), one of R³, R⁴, and R⁵, is independently deuterium. In another embodiment of Formula (VI), two of R³, R⁴, and R⁵, are deuterium. In another embodiment of Formula (VI), R³, R⁴, and R⁵ are each deuterium.

In one embodiment of Formula (VI), R⁶, R⁷, R⁸, and R⁹, are each independently hydrogen or deuterium. In another embodiment of Formula (VI), R⁶, R⁷, R⁸, and R⁹, are each hydrogen provided that at least one of R³, R⁴, R⁵, R¹⁰, R¹¹, R¹², R¹³, R¹⁴, R¹⁵, R¹⁶, R^{17A}, R^{17B}, R^{17C}, R^{17D}, R¹⁸, and R²⁰ is deuterium. In one embodiment of Formula (VI), one of R⁶, R⁷, R⁸, and R⁹, is deuterium. In another embodiment of Formula (VI), two of R⁶, R⁷, R⁸, and R⁹, are deuterium. In another embodiment of Formula (VI), three of R⁶, R⁷, R⁸, and R⁹, are deuterium. In another embodiment of Formula (VI), R⁶, R⁷, R⁸, and R⁹ are each deuterium.

In one embodiment of Formula (VI), R¹⁰, R¹⁴, R¹⁵, and R¹⁶, are each independently hydrogen or deuterium. In another embodiment of Formula (VI), R¹⁰, R¹⁴, R¹⁵, and R¹⁶, are each hydrogen provided that at least one of R³, R⁴, R⁵, R⁶, R⁷, R⁸, R⁹, R¹¹, R¹², R¹³, R^{17A}, R^{17B}, R^{17C}, R^{17D}, R¹⁸, and R²⁰ is deuterium. In one embodiment of Formula (VI), one of R¹⁰, R¹⁴, R¹⁵, and R¹⁶, is deuterium. In another embodiment of Formula (VI), two of R¹⁰, R¹⁴, R¹⁵, and R¹⁶, are deuterium. In another embodiment of Formula (VI), three of R¹⁰, R¹⁴, R¹⁵, and R¹⁶, are deuterium. In another embodiment of Formula (VI), R¹⁰, R¹⁴, R¹⁵, and R¹⁶, are deuterium.

In one embodiment of Formula (VI), R¹¹, R¹², and R¹³ are each independently hydrogen or deuterium. In another embodiment of Formula (VI), R¹¹, R¹², and R¹³ are each hydrogen provided that at least one of R³, R⁴, R⁵, R⁶, R⁷, R⁸, R⁹, R¹⁰, R¹⁴, R¹⁵, R¹⁶, R^{17A}, R^{17B}, R^{17C}, R^{17D}, R¹⁸, and R²⁰ is deuterium. In one embodiment of Formula (VI), one of R¹¹, R¹², and R¹³ is deuterium. In another embodiment of Formula (VI), two of R¹¹, R¹², and R¹³ are deuterium. In another embodiment of Formula (VI), R¹¹, R¹², and R¹³ are deuterium.

In one embodiment of Formula (VI), R²⁰ is independently hydrogen, deuterium, or fluorine. In another embodiment of Formula (VI), R²⁰ is hydrogen provided that at least one of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, and R¹⁸ is deuterium. In another embodiment of Formula (VI), R²⁰ is fluorine provided that at least one of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, and R¹⁸ is deuterium. In another embodiment of Formula (VI), R²⁰ is deuterium.

In one embodiment of Formula (VI), R¹⁸ is hydrogen or deuterium. In another embodiment of Formula (VI), R¹⁸ is hydrogen provided that at least one of R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, and R²⁰ is deuterium. In another embodiment of Formula (VI), R¹⁸ is deuterium.

In one embodiment of Formula (VI), R^{17A}, R^{17B}, R^{17C}, and R^{17D} are each independently hydrogen or deuterium. In another embodiment of Formula (VI), R^{17A}, R^{17B}, R^{17C}, and R^{17D} are each hydrogen provided that at least one of R³, R⁴, R⁵, R⁶, R⁷, R⁸, R⁹, R¹⁰, R¹¹, R¹², R¹³, R¹⁴, R¹⁵, R¹⁶, R¹⁸, and R²⁰ is deuterium. In another embodiment of Formula (VI), R^{17A} is deuterium; and R^{17B}, R^{17C}, and R^{17D} are each hydrogen. In another embodiment of Formula (VI), R^{17B} is deuterium; and R^{17A}, R^{17C}, and R^{7D} are each hydrogen. In another embodiment of Formula (VI), R^{17A} and R^{17C} are each deuterium; and R^{17B} and R^{17D} are each hydrogen. In another embodiment of Formula (VI), R^{17B} and R^{17D} are each deuterium; and R^{17A} and R^{17C} are each hydrogen. In another embodiment of Formula (VI), R^{17A} and R^{17B} are each deuterium; and R^{17C} and R^{17D} are each hydrogen. In another embodiment of Formula (VI), R^{17A} and R^{17D} are each deuterium; and R^{17B} and R^{17C} are each hydrogen. In another embodiment of Formula (VI), R^{17A}, R^{17B}, and R^{17C} are each deuterium; and R^{17D} is hydrogen. In another embodiment of Formula (VI), R^{17A}, R^{17C}, and R^{17D} are each deuterium; and R^{17B} is hydrogen. In another embodiment of Formula (VI), R^{17A}, R^{17B}, R^{17C}, and R^{17D} are each deuterium.

One embodiment pertains to compounds of Formula (VI), or pharmaceutically acceptable salts thereof, wherein
R³, R⁴, R⁵, R⁶, R⁷, R⁸, R⁹, R¹³, R¹⁴, R^{17B}, R^{17D}, R¹⁸, and R²⁰ are each independently hydrogen; and R¹⁰, R¹¹, R¹², R¹⁵, R¹⁶, R^{17A}, and R^{17C} are each independently deuterium.

One embodiment pertains to compounds of Formula (VI), or pharmaceutically acceptable salts thereof, wherein
R³, R⁴, R⁵, R¹⁰, R¹¹, R¹², R¹³, R¹⁴, R¹⁵, R¹⁶, R¹⁸, and R²⁰ are each independently hydrogen;
R⁶, R⁷, R⁸, and R⁹ are each independently deuterium; and
R^{17A}, R^{17B}, R^{17C}, R^{17D} are each independently hydrogen.

One embodiment pertains to compounds of Formula (VIa),

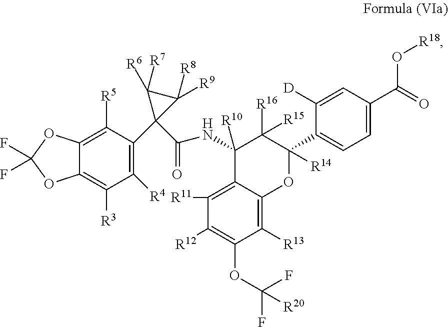

wherein R³, R⁴, R⁵, R⁶, R⁷, R⁸, R⁹, R¹⁰, R¹¹, R¹², R¹³, R¹⁴, R¹⁵, R¹⁶, R¹⁸, and R²⁰ are as described in embodiments of Formula (VI) herein.

One embodiment pertains to compounds of Formula (VIb),

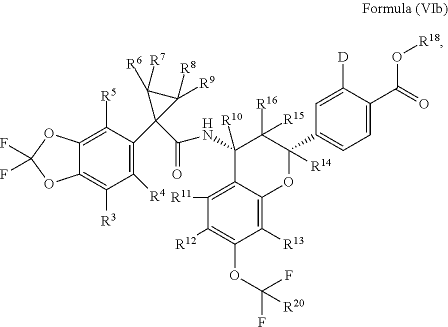

wherein R³, R⁴, R⁵, R⁶, R⁷, R⁸, R⁹, R¹⁰, R¹¹, R¹², R¹³, R¹⁴, R¹⁵, R¹⁶, R¹⁸, and R²⁰ are as described in embodiments of Formula (VI) herein.

One embodiment pertains to compounds of Formula (VIc),

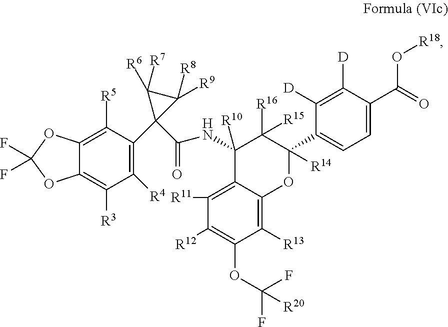

wherein R³, R⁴, R⁵, R⁶, R⁷, R⁸, R⁹, R¹⁰, R¹¹, R¹², R¹³, R¹⁴, R¹⁵, R¹⁶, R¹⁸, and R²⁰ are as described in embodiments of Formula (VI) herein.

One embodiment pertains to compounds of Formula (VId),

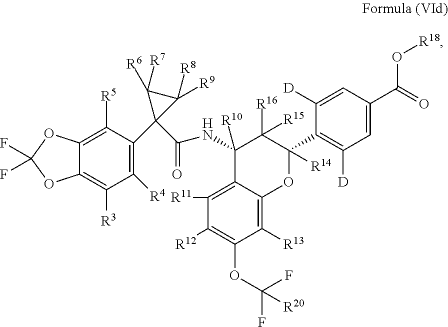

wherein R³, R⁴, R⁵, R⁶, R⁷, R⁸, R⁹, R¹⁰, R¹¹, R¹², R¹³, R¹⁴, R¹⁵, R¹⁶, R¹⁸, and R²⁰ are as described in embodiments of Formula (VI) herein.

One embodiment pertains to compounds of Formula (VIe),

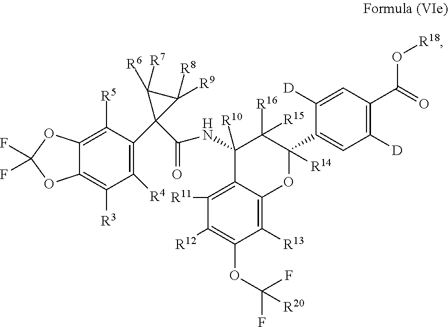

wherein R³, R⁴, R⁵, R⁶, R⁷, R⁸, R⁹, R¹⁰, R¹¹, R¹², R¹³, R¹⁴, R¹⁵, R¹⁶, R¹⁸, and R²⁰ are as described in embodiments of Formula (VI) herein.

One embodiment pertains to compounds of Formula (VIf),

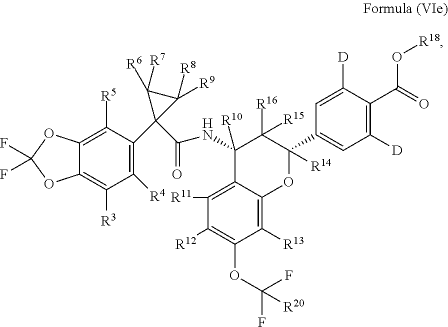

wherein R³, R⁴, R⁵, R⁶, R⁷, R⁸, R⁹, R¹⁰, R¹¹, R¹², R¹³, R¹⁴, R¹⁵, R¹⁶, R¹⁸, and R²⁰ are as described in embodiments of Formula (VI) herein.

One embodiment pertains to compounds of Formula (VIg),

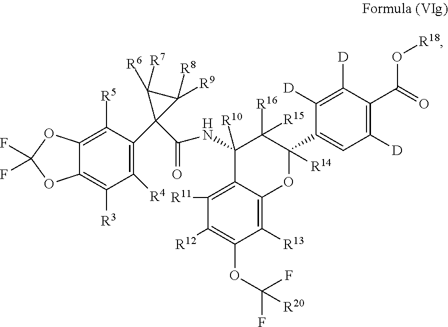

wherein R³, R⁴, R⁵, R⁶, R⁷, R⁸, R⁹, R¹⁰, R¹¹, R¹², R¹³, R¹⁴, R¹⁵, R¹⁶, R¹⁸, and R²⁰ are as described in embodiments of Formula (VI) herein.

One embodiment pertains to compounds of Formula (VIh),

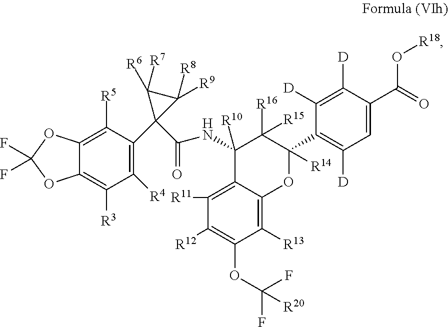

wherein R³, R⁴, R⁵, R⁶, R⁷, R⁸, R⁹, R¹⁰, R¹¹, R¹², R¹³, R¹⁴, R¹⁵, R¹⁶, R¹⁸, and R²⁰ are as described in embodiments of Formula (VI) herein.

One embodiment pertains to compounds of Formula (Vii),

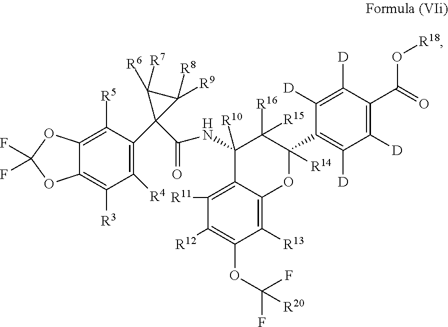

wherein R³, R⁴, R⁵, R⁶, R⁷, R⁸, R⁹, R¹⁰, R¹¹, R¹², R¹³, R¹⁴, R¹⁵, R¹⁶, R¹⁸, and R²⁰ are as described in embodiments of Formula (VI) herein.

Compound names are assigned by using Name 2014 Release (Build 66687) naming algorithm by Advanced Chemical Development or Struct=Name naming algorithm as part of CHEMDRAW® ULTRA v. 12.0.2.1076 or Professional Version 15.0.0.106.

Compounds of the invention may exist as stereoisomers wherein asymmetric or chiral centers are present. These stereoisomers are “R” or “S” depending on the configuration of substituents around the chiral carbon atom. The terms “R” and “S” used herein are configurations as defined in IUPAC 1974 Recommendations for Section E, Fundamental Stereochemistry, in Pure Appl. Chem., 1976, 45: 13-30. The invention contemplates various stereoisomers and mixtures thereof and these are specifically included within the scope of this invention. Stereoisomers include enantiomers and diastereomers, and mixtures of enantiomers or diastereomers. Individual stereoisomers of compounds of the invention may be prepared synthetically from commercially available starting materials which contain asymmetric or chiral centers or by preparation of racemic mixtures followed by methods of resolution well-known to those of ordinary skill in the art. These methods of resolution are exemplified by (1) attachment of a mixture of enantiomers to a chiral auxiliary, separation of the resulting mixture of diastereomers by recrystallization or chromatography and optional liberation of the optically pure product from the auxiliary as described in Furniss, Hannaford, Smith, and Tatchell, “Vogel's Textbook of Practical Organic Chemistry”, 5th edition (1989), Longman Scientific & Technical, Essex CM20 2JE, England, or (2) direct separation of the mixture of optical enantiomers on chiral chromatographic columns or (3) fractional recrystallization methods.

Compounds of the invention may exist as cis or trans isomers, wherein substituents on a ring may attached in such a manner that they are on the same side of the ring (cis) relative to each other, or on opposite sides of the ring relative to each other (trans). For example, cyclobutane may be present in the cis or trans configuration, and may be present as a single isomer or a mixture of the cis and trans isomers. Individual cis or trans isomers of compounds of the invention may be prepared synthetically from commercially available starting materials using selective organic transformations, or prepared in single isomeric form by purification of mixtures of the cis and trans isomers. Such methods are well-known to those of ordinary skill in the art, and may include separation of isomers by recrystallization or chromatography.

It should be understood that the compounds of the invention may possess tautomeric forms, as well as geometric isomers, and that these also constitute an aspect of the invention.

It should be understood that hydrogen has three naturally occurring isotopes, deuterium occurs naturally on earth, and the amount of naturally occurring deuterium on earth is very small (approximately 0.0156%). Therefore, compounds with designated hydrogens at specific positions contain very small amounts of naturally occurring deuterium at its natural isotopic quantity. Thus, it should also be understood that the compounds of this invention naturally contain small amounts of many unspecified isotopologues. It should be understood that, unless otherwise stated, compounds with a designated hydrogen in a specific position contain hydrogen at its natural isotopic composition.

Similarly, compounds with a designated deuterium at a specific position also contain hydrogen at that position; the exact amount depends on the isotopic enrichment factor. It should also be understood that, unless otherwise stated, compounds with designated deuterium in a specific position contain deuterium at an abundance of at least 3205 (50%) of its natural isotopic composition.

In one embodiment, the isotopic enrichment factor for each labeled deuterium in compounds of the invention is greater than 3205 (greater than 50%). In another embodiment, the isotopic enrichment factor for each labeled deuterium in compounds of the invention is 3526 or more (greater or equal to 55%). In another embodiment, the isotopic enrichment factor for each labeled deuterium in compounds of the invention is 3846 or more (greater or equal to 60%). In another embodiment, the isotopic enrichment factor for each labeled deuterium in compounds of the invention is 4167 or more (greater or equal to 65%). In another embodiment, the isotopic enrichment factor for each labeled deuterium in compounds of the invention is 4487 or more (greater or equal to 70%). In another embodiment, the isotopic enrichment factor for each labeled deuterium in compounds of the invention is 4808 or more (greater or equal to 75%). In another embodiment, the isotopic enrichment factor for each labeled deuterium in compounds of the invention is 5128 or more (greater or equal to 80%). In another embodiment, the isotopic enrichment factor for each labeled deuterium in compounds of the invention is 5449 or more (greater or equal to 85%). In another embodiment, the isotopic enrichment factor for each labeled deuterium in compounds of the invention is 5769 or more (greater or equal to 90%). In another embodiment, the isotopic enrichment factor for each labeled deuterium in compounds of the invention is 6090 or more (greater or equal to 95%). In another embodiment, the isotopic enrichment factor for each labeled deuterium in compounds of the invention is 6218 or more (greater or equal to 97%). In another embodiment, the isotopic enrichment factor for each labeled deuterium in compounds of the invention is 6282 or more (greater or equal to 98%). In another embodiment, the isotopic enrichment factor for each labeled deuterium in compounds of the invention is 6346 or more (greater or equal to 99%).

It should be understood that the isotopic enrichment factor and amount of each isotopologue is dependent on several factors including: the natural abundance of deuterium, the purity of deuterated reagents used in the synthesis, and the effectiveness of the synthesis used to incorporate deuterium into the compounds.

The present disclosure includes additional pharmaceutically acceptable isotopically-labelled compounds of Formula (I) wherein one or more atoms are replaced by atoms having the same atomic number, but an atomic mass or mass number different from the atomic mass or mass number which predominates in nature. Examples of additional isotopes suitable for inclusion in the compounds of the disclosure include isotopes of carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S. Certain isotopically-labelled compounds of Formula (I) for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection. Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of Formula (I) may generally be prepared by conventional techniques known to those skilled in the art or by processes analogous to those described in the accompanying Examples using an appropriate isotopically-labeled reagents in place of the non-labeled reagent previously employed.

Thus, the formula drawings within this specification can represent only one of the possible tautomeric, geometric, or stereoisomeric forms. It is to be understood that the invention encompasses any tautomeric, geometric, or stereoisomeric form, and mixtures thereof, and is not to be limited merely to any one tautomeric, geometric, or stereoisomeric form utilized within the formula drawings.

Compounds of Formula (I) may be used in the form of pharmaceutically acceptable salts. The phrase “pharmaceutically acceptable salt” means those salts which are, within the scope of sound medical judgement, suitable for use in contact with the tissues of humans and lower animals without undue toxicity, irritation, allergic response and the like and are commensurate with a reasonable benefit/risk ratio.

Pharmaceutically acceptable salts have been described in S. M. Berge et al. J. Pharmaceutical Sciences, 1977, 66: 1-19.

Compounds of Formula (I) may contain either a basic or an acidic functionality, or both, and can be converted to a pharmaceutically acceptable salt, when desired, by using a suitable acid or base. The salts may be prepared in situ during the final isolation and purification of the compounds of the invention.

Examples of acid addition salts include, but are not limited to acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, fumarate, hydrochloride, hydrobromide, hydroiodide, 2-hydroxyethansulfonate (isothionate), lactate, malate, maleate, methanesulfonate, nicotinate, 2-naphthalenesulfonate, oxalate, palmitoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups may be quaternized with such agents as lower alkyl halides such as, but not limited to, methyl, ethyl, propyl, and butyl chlorides, bromides and iodides; dialkyl sulfates like dimethyl, diethyl, dibutyl and diamyl sulfates; long chain halides such as, but not limited to, decyl, lauryl, myristyl and stearyl chlorides, bromides and iodides; arylalkyl halides like benzyl and phenethyl bromides and others. Water or oil-soluble or dispersible products are thereby obtained. Examples of acids which may be employed to form pharmaceutically acceptable acid addition salts include such inorganic acids as hydrochloric acid, hydrobromic acid, sulfuric acid, and phosphoric acid and such organic acids as acetic acid, fumaric acid, maleic acid, 4-methylbenzenesulfonic acid, succinic acid, and citric acid.

Basic addition salts may be prepared in situ during the final isolation and purification of compounds of this invention by reacting a carboxylic acid-containing moiety with a suitable base such as, but not limited to, the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as, but not limited to, lithium, sodium, potassium, calcium, magnesium and aluminum salts and the like and nontoxic quaternary ammonia and amine cations including ammonium, tetramethylammonium, tetraethylammonium, methylamine, dimethylamine, trimethylamine, triethylamine, diethylamine, ethylamine and the like. Other examples of organic amines useful for the formation of base addition salts include ethylenediamine, ethanolamine, diethanolamine, piperidine, piperazine and the like.

The term “pharmaceutically acceptable prodrug” or “prodrug” as used herein, refers to derivatives of the compounds of the invention which have cleavable groups. Such derivatives become, by solvolysis or under physiological conditions, the compounds of the invention which are pharmaceutically active in vivo. Prodrugs of the compounds of the invention are, within the scope of sound medical judgement, suitable for use in contact with the tissues of humans and lower animals without undue toxicity, irritation, allergic response, and the like, commensurate with a reasonable benefit/risk ratio, and effective for their intended use.

The invention contemplates compounds of Formula (I) formed by synthetic means or formed by in vivo biotransformation of a prodrug.

Compounds described herein may exist in unsolvated as well as solvated forms, including hydrated forms, such as hemi-hydrates. In general, the solvated forms, with pharmaceutically acceptable solvents such as water and ethanol among others are equivalent to the unsolvated forms for the purposes of the invention.

### Pharmaceutical Compositions

When employed as a pharmaceutical, a compound of the invention is typically administered in the form of a pharmaceutical composition. Such compositions can be prepared in a manner well known in the pharmaceutical art and comprise a therapeutically effective amount of a compound of Formula (I), or a pharmaceutically acceptable salt thereof together with a pharmaceutically acceptable carrier. The phrase “pharmaceutical composition” refers to a composition suitable for administration in medical or veterinary use.

The pharmaceutical compositions that comprise a compound of Formula (I), alone or in combination with further therapeutically active ingredient, may be administered to the subjects orally, rectally, parenterally, intracisternally, intravaginally, intraperitoneally, topically (as by powders, ointments or drops), bucally or as an oral or nasal spray. The term “parenterally” as used herein, refers to modes of administration which include intravenous, intramuscular, intraperitoneal, intrasternal, subcutaneous and intraarticular injection and infusion.

The term “pharmaceutically acceptable carrier” as used herein, means a non-toxic, inert solid, semi-solid or liquid filler, diluent, encapsulating material or formulation auxiliary of any type. Some examples of materials which may serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such a propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants may also be present in the composition, according to the judgment of the formulator.

Pharmaceutical compositions for parenteral injection comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous diluents, solvents, or vehicles include water, ethanol, polyols (such as glycerol, propylene glycol, polyethylene glycol and the like), vegetable oils (such as olive oil), injectable organic esters (such as ethyl oleate), and suitable mixtures thereof. Proper fluidity may be maintained, for example, by the use of coating materials such as lecithin, by the maintenance of the required particle size in the case of dispersions and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents which delay absorption, such as aluminum monostearate and gelatin.

In some cases, in order to prolong the effect of the drug, it may be desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally-administered drug form may be accomplished by dissolving or suspending the drug in an oil vehicle.

Injectable depot forms are made by forming microencapsule matrices of the drug in biodegradable polymers such as polylactide-polyglycolide. Depending upon the ratio of drug to polymer and the nature of the particular polymer employed, the rate of drug release may be controlled. Examples of other biodegradable polymers include poly(orthoesters) and poly(anhydrides). Depot injectable formulations are also prepared by entrapping the drug in liposomes or microemulsions which are compatible with body tissues.

The injectable formulations may be sterilized, for example, by filtration through a bacterial-retaining filter or by incorporating sterilizing agents in the form of sterile solid compositions which can be dissolved or dispersed in sterile water or other sterile injectable medium just prior to use.

Solid dosage forms for oral administration include capsules, tablets, pills, powders, and granules. In certain embodiments, solid dosage forms may contain from 1% to 95% (w/w) of a compound of Formula (I). In certain embodiments, the compound of Formula (I), or pharmaceutically acceptable salts thereof, may be present in the solid dosage form in a range of from 5% to 70% (w/w). In such solid dosage forms, the active compound may be mixed with at least one inert, pharmaceutically acceptable carrier, such as sodium citrate or dicalcium phosphate and/or a), fillers or extenders such as starches, lactose, sucrose, glucose, mannitol, and silicic acid; b) binders such as carboxymethylcellulose, alginates, gelatin, polyvinylpyrrolidone, sucrose, and acacia; c) humectants such as glycerol; d) disintegrating agents such as agar-agar, calcium carbonate, potato or tapioca starch, alginic acid, certain silicates, and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay and i) lubricants such as talc, calcium stearate, magnesium stearate, solid polyethylene glycols, sodium lauryl sulfate and mixtures thereof. In the case of capsules, tablets and pills, the dosage form may also comprise buffering agents.

The pharmaceutical composition may be a unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampules. Also, the unit dosage form may be a capsule, tablet, cachet, or lozenge itself, or it may be the appropriate number of any of these in packaged form. The quantity of active component in a unit dose preparation may be varied or adjusted from 0.1 mg to 1000 mg, from 1 mg to 100 mg, or from 1% to 95% (w/w) of a unit dose, according to the particular application and the potency of the active component. The composition may, if desired, also contain other compatible therapeutic agents.

The dose to be administered to a subject may be determined by the efficacy of the particular compound employed and the condition of the subject, as well as the body weight or surface area of the subject to be treated. The size of the dose also will be determined by the existence, nature, and extent of any adverse side-effects that accompany the administration of a particular compound in a particular subject. In determining the effective amount of the compound to be administered in the treatment or prophylaxis of the disorder being treated, the physician may evaluate factors such as the circulating plasma levels of the compound, compound toxicities, and/or the progression of the disease, etc.

For administration, compounds may be administered at a rate determined by factors that may include, but are not limited to, the LD₅₀ of the compound, the pharmacokinetic profile of the compound, contraindicated drugs, and the side-effects of the compound at various concentrations, as applied to the mass and overall health of the subject. Administration may be accomplished via single or divided doses.

The compounds utilized in the pharmaceutical method of the invention may be administered at the initial dosage of about 0.001 mg/kg to about 100 mg/kg daily. In certain embodiments, the daily dose range is from about 0.1 mg/kg to about 10 mg/kg. The dosages, however, may be varied depending upon the requirements of the subject, the severity of the condition being treated, and the compound being employed. Determination of the proper dosage for a particular situation is within the skill of the practitioner. Treatment may be initiated with smaller dosages, which are less than the optimum dose of the compound. Thereafter, the dosage is increased by small increments until the optimum effect under circumstances is reached. For convenience, the total daily dosage may be divided and administered in portions during the day, if desired.

Solid compositions of a similar type may also be employed as fillers in soft and hard-filled gelatin capsules using such carriers as lactose or milk sugar as well as high molecular weight polyethylene glycols and the like.

The solid dosage forms of tablets, dragees, capsules, pills and granules can be prepared with coatings and shells such as enteric coatings and other coatings well-known in the pharmaceutical formulating art. They may optionally contain opacifying agents and may also be of a composition such that they release the active ingredient(s) only, or preferentially, in a certain part of the intestinal tract, optionally, in a delayed manner. Examples of embedding compositions which can be used include polymeric substances and waxes.

The active compounds may also be in micro-encapsulated form, if appropriate, with one or more of the above-mentioned carriers.

Liquid dosage forms for oral administration include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor and sesame oils), glycerol, tetrahydrofurfuryl alcohol, polyethylene glycols, and fatty acid esters of sorbitan and mixtures thereof.

Besides inert diluents, the oral compositions may also include adjuvants such as wetting agents, emulsifying and suspending agents, sweetening, flavoring and perfuming agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar, tragacanth and mixtures thereof.

Compositions for rectal or vaginal administration are preferably suppositories which may be prepared by mixing the compounds with suitable non-irritating carriers or carriers such as cocoa butter, polyethylene glycol, or a suppository wax which are solid at room temperature but liquid at body temperature and therefore melt in the rectum or vaginal cavity and release the active compound.

Compounds may also be administered in the form of liposomes. Liposomes generally may be derived from phospholipids or other lipid substances. Liposomes are formed by mono- or multi-lamellar hydrated liquid crystals which are dispersed in an aqueous medium. Any non-toxic, physiologically acceptable and metabolizable lipid capable of forming liposomes may be used. The present compositions in liposome form may contain, in addition to a compound of the invention, stabilizers, preservatives, excipients, and the like. Examples of lipids include, but are not limited to, natural and synthetic phospholipids, and phosphatidyl cholines (lecithins), used separately or together.

Methods to form liposomes have been described, see example, Prescott, Ed., Methods in Cell Biology, Volume XIV, Academic Press, New York, N.Y. (1976), p. 33 et seq.

Dosage forms for topical administration of a compound described herein include powders, sprays, ointments, and inhalants. The active compound may be mixed under sterile conditions with a pharmaceutically acceptable carrier and any needed preservatives, buffers or propellants which may be required. Opthalmic formulations, eye ointments, powders and solutions are also contemplated as being within the scope of this invention.

A compound of the invention may also be administered in sustained release forms or from sustained release drug delivery systems.

### Methods of Use

The compounds and compositions using any amount and any route of administration may be administered to a subject for the treatment or prevention of cystic fibrosis, pancreatic insufficiency, Sjögren's syndrome (SS), chronic obstructive lung disease (COLD), or chronic obstructive airway disease (COAD).

The term “administering” refers to the method of contacting a compound with a subject. Thus, the compounds may be administered by injection, that is, intravenously, intramuscularly, intracutaneously, subcutaneously, intraduodenally, parentally, or intraperitoneally. Also, the compounds described herein may be administered by inhalation, for example, intranasally. Additionally, the compounds may be administered transdermally, topically, and via implantation. In certain embodiments, the compounds and compositions thereof may be delivered orally. The compounds may also be delivered rectally, bucally, intravaginally, ocularly, or by insufflation. CFTR-modulated disorders and conditions may be treated prophylactically, acutely, and chronically using compounds and compositions thereof, depending on the nature of the disorder or condition. Typically, the host or subject in each of these methods is human, although other mammals may also benefit from the administration of compounds and compositions thereof as set forth hereinabove.

Compounds of the invention are useful as modulators of CFTR. Thus, the compounds and compositions are particularly useful for treating or lessening the severity or progression of a disease, disorder, or a condition where hyperactivity or inactivity of CFTR is involved. Accordingly, the invention provides a method for treating cystic fibrosis, pancreatic insufficiency, Sjögren's syndrome (SS), chronic obstructive lung disease (COLD), or chronic obstructive airway disease (COAD) in a subject, wherein the method comprises the step of administering to said subject a therapeutically effective amount of a compound of Formula (I), or a pharmaceutically acceptable salt thereof, or a preferred embodiment thereof as set forth above, with or without a pharmaceutically acceptable carrier. Particularly, the method is for the treatment or prevention of cystic fibrosis. In a more particular embodiment, the cystic fibrosis is caused by a Class I, II, III, IV, V, and/or VI mutation.

In a particular embodiment, the present invention provides compounds of the invention, or a pharmaceutically acceptable salt thereof, or a pharmaceutical composition comprising a compound of the invention, for use in medicine. In a particular embodiment, the present invention provides compounds of the invention, or a pharmaceutically acceptable salt thereof, or pharmaceutical compositions comprising a compound of the invention, for use in the treatment of cystic fibrosis, pancreatic insufficiency, Sjögren's syndrome (SS), chronic obstructive lung disease (COLD) or chronic obstructive airway disease (COAD). In a more particular embodiment, the present invention provides compounds of the invention or pharmaceutical compositions comprising a compound of the invention, for use in the treatment of cystic fibrosis. In a more particular embodiment, the cystic fibrosis is caused by a Class I, II, III, IV, V, and/or VI mutation.

One embodiment is directed to the use of a compound according to Formula (I) or a pharmaceutically acceptable salt thereof in the preparation of a medicament. The medicament optionally can comprise one or more additional therapeutic agents. In some embodiments, the medicament is for use in the treatment of cystic fibrosis, pancreatic insufficiency, Sjögren's syndrome (SS), chronic obstructive lung disease (COLD) or chronic obstructive airway disease (COAD). In a particular embodiment, the medicament is for use in the treatment of cystic fibrosis. In a more particular embodiment, the cystic fibrosis is caused by a Class I, II, III, IV, V, and/or VI mutation.

This invention also is directed to the use of a compound according to Formula (I) or a pharmaceutically acceptable salt thereof in the manufacture of a medicament for the treatment of cystic fibrosis, Sjögren's syndrome, pancreatic insufficiency, chronic obstructive lung disease, and chronic obstructive airway disease. The medicament optionally can comprise one or more additional therapeutic agents. In a particular embodiment, the invention is directed to the use of a compound according to Formula (I) or a pharmaceutically acceptable salt thereof in the manufacture of a medicament for the treatment of cystic fibrosis. In a more particular embodiment, the cystic fibrosis is caused by a Class I, II, III, IV, V, and/or VI mutation.

In one embodiment, the present invention provides pharmaceutical compositions comprising a compound of the invention, or a pharmaceutically acceptable salt thereof, and one or more additional therapeutic agents. In another embodiment, the present invention provides pharmaceutical compositions comprising a compound of the invention, or a pharmaceutically acceptable salt thereof, and one or more additional therapeutic agents wherein the additional therapeutic agents are selected from the group consisting of CFTR modulators and CFTR amplifiers. In another embodiment, the present invention provides pharmaceutical compositions comprising a compound of the invention, or a pharmaceutically acceptable salt thereof, and one or more additional therapeutic agents wherein the additional therapeutic agents are CFTR modulators.

In one embodiment, the present invention provides pharmaceutical compositions comprising a compound of the invention, or a pharmaceutically acceptable salt thereof, and one or more additional therapeutic agents. In one embodiment, the present invention provides pharmaceutical compositions comprising a compound of the invention, or a pharmaceutically acceptable salt thereof, one potentiator, and one or more additional correctors. In one embodiment, the present invention provides pharmaceutical compositions comprising a compound of the invention, and another therapeutic agent. In a particular embodiment, the other therapeutic agent is a cystic fibrosis treatment agent. In one embodiment, the present invention provides a method for treating cystic fibrosis in a subject comprising administering a compound of the invention, or a pharmaceutically acceptable salt thereof, and one or more additional therapeutic agents. In another embodiment, the present invention provides a method for treating cystic fibrosis in a subject comprising administering a compound of the invention, or a pharmaceutically acceptable salt thereof, and one or more additional therapeutic agents wherein the additional therapeutic agents are selected from the group consisting of CFTR modulators and CFTR amplifiers. In one embodiment, the present invention provides a method for treating cystic fibrosis in a subject comprising administering a compound of the invention, or a pharmaceutically acceptable salt thereof, and one or more additional therapeutic agents wherein the additional therapeutic agents are CFTR modulators. In one embodiment, the present invention provides a method for treating cystic fibrosis in a subject comprising administering a compound of the invention, or a pharmaceutically acceptable salt thereof, and, and another therapeutic agent. In a particular embodiment, the other therapeutic agent is a cystic fibrosis treatment agent. In one embodiment, the present invention provides a method for treating cystic fibrosis in a subject comprising administering a therapeutically effective amount of a compound of the invention, or a pharmaceutically acceptable salt thereof. In a particular embodiment, the additional therapeutic agent(s) are one potentiator, and one or more additional correctors. In another embodiment, the additional therapeutic agent(s) is selected from the group consisting of CFTR modulators and CFTR amplifiers. In another embodiment, the other therapeutic agent(s) is a CFTR modulator. In a more particular embodiment, the cystic fibrosis is caused by a Class I, II, III, IV, V, and/or VI mutation.

The present compounds or pharmaceutically acceptable salts thereof may be administered as the sole active agent or it may be co-administered with other therapeutic agents, including other compounds or pharmaceutically acceptable salts thereof, that demonstrate the same or a similar therapeutic activity and that are determined to be safe and efficacious for such combined administration. The present compounds may be co-administered to a subject. The term “co-administered” means the administration of two or more different therapeutic agents to a subject in a single pharmaceutical composition or in separate pharmaceutical compositions. Thus co-administration involves administration at the same time of a single pharmaceutical composition comprising two or more therapeutic agents or administration of two or more different compositions to the same subject at the same or different times.

The compounds of the invention or pharmaceutically acceptable salts thereof may be co-administered with a therapeutically effective amount of one or more additional therapeutic agents to treat a CFTR mediated disease, where examples of the therapeutic agents include, but are not limited to antibiotics (for example, aminoglycosides, colistin, aztreonam, ciprofloxacin, and azithromycin), expectorants (for example, hypertonic saline, acetylcysteine, dornase alfa, and denufosol), pancreatic enzyme supplements (for example, pancreatin, and pancrelipase), epithelial sodium channel blocker (ENaC) inhibitors, CFTR modulators (for example, CFTR potentiators, CFTR correctors), and CFTR amplifiers. In one embodiment, the CFTR mediated disease is cystic fibrosis, chronic obstructive pulmonary disease (COPD), dry eye disease, pancreatic insufficiency, or Sjögren's syndrome. In one embodiment, the CFTR mediated disease is cystic fibrosis. In one embodiment, the compounds of the invention or pharmaceutically acceptable salts thereof may be co-administered with one or two CFTR modulators and one CFTR amplifier. In one embodiment, the compounds of the invention or pharmaceutically acceptable salts thereof may be co-administered with one potentiator, one or more correctors, and one CFTR amplifier. In one embodiment, the compounds of the invention or pharmaceutically acceptable salts thereof may be co-administered with one or more CFTR modulators. In one embodiment, the compounds of the invention or pharmaceutically acceptable salts thereof may be co-administered with one CFTR modulators. In one embodiment, the compounds of the invention or pharmaceutically acceptable salts thereof may be co-administered with two CFTR modulators. In one embodiment, the compounds of the invention or pharmaceutically acceptable salts thereof may be co-administered with three CFTR modulators. In one embodiment, the compounds of the invention or pharmaceutically acceptable salts thereof may be co-administered with one potentiator and one or more correctors. In one embodiment, the compounds of the invention or pharmaceutically acceptable salts thereof may be co-administered with one potentiator and two correctors. In one embodiment, the compounds of the invention or pharmaceutically acceptable salts thereof may be co-administered with one potentiator. In one embodiment, the compounds of the invention or pharmaceutically acceptable salts thereof may be co-administered with one or more correctors. In one embodiment, the compounds of the invention or pharmaceutically acceptable salts thereof may be co-administered with one corrector. In one embodiment, the compounds of the invention or pharmaceutically acceptable salts thereof may be co-administered with two correctors.

Examples of CFTR potentiators include, but are not limited to, Ivacaftor (VX-770), CTP-656, NVS-QBW251. FD 1860293, GLPG2451, GLPG1837, PTI-808, N-(3-carbamoyl-5,5,7,7-tetramethyl-5,7-dihydro-4H-thieno[2,3-c]pyran-2-yl)-1H-pyrazole-5-carboxamide, and 3-amino-N-[(2S)-2-hydroxypropyl]-5-{[4-(trifluoromethoxy)phenyl]sulfonyl}pyridine-2-carboxamide. Examples of potentiators are also disclosed in publications: WO2005120497, WO2008147952, WO2009076593, WO2010048573, WO2006002421, WO2008147952, WO2011072241, WO2011113894, WO2013038373, WO2013038378, WO2013038381, WO2013038386, WO2013038390, WO2014/180562, WO2015018823, WO2016193812 and WO2017208115.

In one embodiment, the potentiator can be selected from the group consisting of
Ivacaftor (VX-770, N-(2,4-di-tert-butyl-5-hydroxyphenyl)-4-oxo-1,4-dihydroquinoline-3-carboxamide);
GLPG1837;
GLPG2451;
PTI-808;
CTP-656;
NVS-QBW251;
FD1860293;
2-(2-fluorobenzamido)-5,5,7,7-tetramethyl-5,7-dihydro-4H-thieno[2,3-c]pyran-3-carboxamide; N-(3-carbamoyl-5,5,7,7-tetramethyl-4,7-dihydro-5H-thieno[2,3-c]pyran-2-yl)-1H-pyrazole-5-carboxamide;
2-(2-hydroxybenzamido)-5,5,7,7-tetramethyl-5,7-dihydro-4H-thieno[2,3-c]pyran-3-carboxamide
2-(1-hydroxycyclopropanecarboxamido)-5,5,7,7-tetramethyl-5,7-dihydro-4H-thieno[2,3-c]pyran-3-carboxamide;
5,5,7,7-tetramethyl-2-(2-(trifluoromethyl)benzamido)-5,7-dihydro-4H-thieno[2,3-c]pyran-3-carboxamide;
2-(2-hydroxy-2-methylpropanamido)-5,5,7,7-tetramethyl-5,7-dihydro-4H-thieno[2,3-c]pyran-3-carboxamide;
2-(1-(hydroxymethyl)cyclopropanecarboxamido)-5,5,7,7-tetramethyl-5,7-dihydro-4H-thieno[2,3-c]pyran-3-carboxamide;
2-(3-hydroxy-2,2-dimethylpropanamido)-5,5,7,7-tetramethyl-5,7-dihydro-4H-thieno[2,3-c]pyran-3-carboxamide;
N-(3-carbamoyl-5,5,7,7-tetramethyl-5,7-dihydro-4H-thieno[2,3-c]pyran-2-yl)-5-methyl-1H-pyrazole-3-carboxamide;
N-(3-carbamoyl-5,5,7,7-tetramethyl-5,7-dihydro-4H-thieno[2,3-c]pyran-2-yl)-5-cyclopropyl-1H-pyrazole-3-carboxamide;
N-(3-carbamoyl-5,5,7,7-tetramethyl-5,7-dihydro-4H-thieno[2,3-c]pyran-2-yl)-5-isopropyl-1H-pyrazole-3-carboxamide;
N-(3-carbamoyl-5,5,7,7-tetramethyl-5,7-dihydro-4H-thieno[2,3-c]pyran-2-yl)-5-(trifluoromethyl)-1H-pyrazole-3-carboxamide;
5-tert-butyl-N-(3-carbamoyl-5,5,7,7-tetramethyl-5,7-dihydro-4H-thieno [2,3-c]pyran-2-yl)-1H-pyrazole-3-carboxamide;
N-(3-carbamoyl-5,5,7,7-tetramethyl-5,7-dihydro-4H-thieno[2,3-c]pyran-2-yl)-5-ethyl-1H-pyrazole-3-carboxamide;
N-(3-carbamoyl-5,5,7,7-tetramethyl-5,7-dihydro-4H-thieno[2,3-c]pyran-2-yl)-3-ethyl-4-methyl-1H-pyrazole-5-carboxamide;
2-(2-hydroxypropanamido)-5,5,7,7-tetramethyl-5,7-dihydro-4H-thieno [2,3-c]pyran-3-carboxamide;
N-(3-carbamoyl-5,5,7,7-tetramethyl-5,7-dihydro-4H-thieno[2,3-c]pyran-2-yl)-4-chloro-1H-pyrazole-3-carboxamide;
N-(3-carbamoyl-5,5,7,7-tetramethyl-5,7-dihydro-4H-thieno[2,3-c]pyran-2-yl)-1,4,6,7-tetrahydropyrano[4,3-c]pyrazole-3-carboxamide;
4-bromo-N-(3-carbamoyl-5,5,7,7-tetramethyl-5,7-dihydro-4H-thieno [2,3-c]pyran-2-yl)-1H-pyrazole-3-carboxamide;
N-(3-carbamoyl-5,5,7,7-tetramethyl-5,7-dihydro-4H-thieno[2,3-c]pyran-2-yl)-4-chloro-5-methyl-1H-pyrazole-3-carboxamide;
N-(3-carbamoyl-5,5,7,7-tetramethyl-5,7-dihydro-4H-thieno[2,3-c]pyran-2-yl)-4-methyl-1H-pyrazole-3-carboxamide;
2-(2-hydroxy-3,3-dimethylbutanamido)-5,5,7,7-tetramethyl-5,7-dihydro-4H-thieno [2,3-c]pyran-3-carboxamide;
2-[(2-hydroxy-4-methyl-pentanoyl)amino]-5,5,7,7-tetramethyl-4H-thieno [2,3-c]pyran-3-carboxamide;
5-(2-methoxy-ethoxy)-1H-pyrazole-3-carboxylic acid (3-carbamoyl-5,5,7,7-tetramethyl-4,7-dihydro-5H-thieno [2,3-c]pyran-2-yl)-amide;
N-(3-carbamoyl-5,5,7,7-tetramethyl-4H-thieno [2,3-c]pyran-2-yl)-4-(3-methoxypropyl)-1H-pyrazole-3-carboxamide;
N-(3-carbamoyl-5,5,7,7-tetramethyl-4H-thieno [2,3-c]pyran-2-yl)-4-(2-ethoxyethyl)-1H-pyrazole-3-carboxamide;
2-[[(2S)-2-hydroxy-3,3-dimethyl-butanoyl]amino]-5,5,7,7-tetramethyl-4H-thieno [2,3-c]pyran-3-carboxamide;
2-[[(2R)-2-hydroxy-3,3-dimethyl-butanoyl]amino]-5,5,7,7-tetramethyl-4H-thieno [2,3-c]pyran-3-carboxamide;
2-[(2-hydroxy-2,3,3-trimethyl-butanoyl)amino]-5,5,7,7-tetramethyl-4H-thieno [2,3-c]pyran-3-carboxamide;
[5-[(3-carbamoyl-5,5,7,7-tetramethyl-4H-thieno [2,3-c]pyran-2-yl)carbamoyl]pyrazol-1-yl]methyl dihydrogen phosphate;
[3-[(3-carbamoyl-5,5,7,7-tetramethyl-4H-thieno [2,3-c]pyran-2-yl)carbamoyl]pyrazol-1-yl]methyl dihydrogen phosphate;
N-(3-carbamoyl-5,5,7,7-tetramethyl-4H-thieno [2,3-c]pyran-2-yl)-4-(1,4-dioxan-2-yl)-1H-pyrazole-3-carboxamide;
5,5,7,7-tetramethyl-2-[[(2S)-3,3,3-trifluoro-2-hydroxy-2-methyl-propanoyl]amino]-4H-thieno[2,3-c]pyran-3-carboxamide;
2-[[(2S)-2-hydroxypropanoyl]amino]-5,5,7,7-tetramethyl-4H-thieno [2,3-c]pyran-3-carboxamide;
3-amino-N-(2-hydroxy-2-methylpropyl)-5-{[4-(trifluoromethoxy)phenyl]sulfonyl}pyridine-2-carboxamide;
3-amino-N-[(4-hydroxy-1-methylpiperidin-4-yl)methyl]-5-{[4-(trifluoromethoxy)phenyl]sulfonyl}pyridine-2-carboxamide;
3-amino-N-(3-hydroxy-2,2-dimethylpropyl)-5-{[4-(trifluoromethoxy)phenyl]sulfonyl}pyridine-2-carboxamide;
3-amino-5-[(4-fluorophenyl)sulfonyl]-N-[(1-hydroxycyclopropyl)methyl]pyridine-2-carboxamide;
3-amino-5-[(4-fluorophenyl)sulfonyl]-N-[(2R)-3,3,3-trifluoro-2-hydroxypropyl]pyridine-2-carboxamide;
3-amino-5-[(3-fluorophenyl) sulfonyl]-N-(2-hydroxy-2-methylpropyl)pyridine-2-carboxamide;
3-amino-N-[2-(cyclopropylamino)-2-oxoethyl]-5-{[4-(trifluoromethoxy)phenyl]sulfonyl}pyridine-2-carboxamide;
(3-amino-5-{[4-(trifluoromethoxy)phenyl]sulfonyl}pyridin-2-yl)(azetidin-1-yl)methanone;
(3-amino-5-{[4-(trifluoromethoxy)phenyl]sulfonyl}pyridin-2-yl)[3-(hydroxymethyl)azetidin-1-yl]methanone;
(3-amino-5-{[4-(trifluoromethoxy)phenyl]sulfonyl}pyridin-2-yl)(3-fluoroazetidin-1-yl)methanone;
3-amino-N-[(2R)-2-hydroxy-3-methoxypropyl]-5-{[4-(trifluoromethyl)phenyl]sulfonyl}pyridine-2-carboxamide;
(3-amino-5-{[2-fluoro-4-(trifluoromethoxy)phenyl]sulfonyl}pyridin-2-yl)(3-hydroxyazetidin-1-yl)methanone;
(3-amino-5-{[2-(trifluoromethoxy)phenyl]sulfonyl}pyridin-2-yl)(3,3-difluoroazetidin-1-yl)methanone;
rac-3-amino-N-[(3R,4S)-4-hydroxytetrahydro-2H-pyran-3-yl]-5-{[2-(trifluoromethoxy)phenyl]sulfonyl}pyridine-2-carboxamide;
3-amino-5-[(4,4-difluoropiperidin-1-yl)sulfonyl]-N-(3,3,3-trifluoro-2-hydroxypropyl)pyridine-2-carboxamide;
(3-amino-5-{[2-(trifluoromethoxy)phenyl]sulfonyl}pyridin-2-yl)[3-hydroxy-3-(trifluoromethyl)azetidin-1-yl]methanone;
3-amino-N-(2-hydroxy-4-methylpentyl)-5-{[4-(trifluoromethoxy)phenyl]sulfonyl}pyridine-2-carboxamide;
(3-amino-5-{[4-(trifluoromethyl)phenyl]sulfonyl}pyridin-2-yl)(3-hydroxy-3-methylazetidin-1-yl)methanone;
3-amino-N-(3,3,3-trifluoro-2-hydroxypropyl)-5-{[4-(trifluoromethyl)piperidin-1-yl]sulfonyl}pyridine-2-carboxamide;
3-amino-N-[2-hydroxy-1-(4-methoxyphenyl)ethyl]-5-{[4-(trifluoromethoxy)phenyl]sulfonyl}pyridine-2-carboxamide;
3-amino-5-[(3,3-difluoroazetidin-1-yl)sulfonyl]-N-(3,3,3-trifluoro-2-hydroxypropyl)pyridine-2-carboxamide;
3-amino-5-{[2-fluoro-4-(trifluoromethyl)phenyl]sulfonyl}-N-[(2S)-2-hydroxypropyl]pyridine-2-carboxamide;
3-amino-5-{[2-fluoro-4-(trifluoromethyl)phenyl]sulfonyl}-N-[(2R)-2-hydroxy-3-methoxypropyl]pyridine-2-carboxamide;
3-amino-N-[2-oxo-2-(propan-2-ylamino)ethyl]-5-{[4-(trifluoromethyl)phenyl]sulfonyl}pyridine-2-carboxamide;
(3-amino-5-{[4-(trifluoromethyl)phenyl]sulfonyl}pyridin-2-yl)[3-hydroxy-3-(trifluoromethyl)azetidin-1-yl]methanone;
3-amino-5-{[2-fluoro-4-(trifluoromethyl)phenyl]sulfonyl}-N-[(3R)-tetrahydrofuran-3-ylmethyl]pyridine-2-carboxamide;
(3-amino-5-{[2-fluoro-4-(trifluoromethyl)phenyl]sulfonyl}pyridin-2-yl)[3-hydroxy-3-(trifluoromethyl)azetidin-1-yl]methanone;
3-amino-5-{[2-fluoro-4-(trifluoromethyl)phenyl]sulfonyl}-N-[(3S)-tetrahydrofuran-3-ylmethyl]pyridine-2-carboxamide;
3-amino-5-{[2-fluoro-4-(trifluoromethoxy)phenyl]sulfonyl}-N-[(3S)-tetrahydrofuran-3-ylmethyl]pyridine-2-carboxamide;
3-amino-N-[2-hydroxy-3-(2,2,2-trifluoroethoxy)propyl]-5-{[4-(trifluoromethyl)phenyl]sulfonyl}pyridine-2-carboxamide;
3-amino-N-(3-tert-butoxy-2-hydroxypropyl)-5-{[2-fluoro-4-(trifluoromethyl)phenyl]sulfonyl}pyridine-2-carboxamide;
[3-amino-5-(phenylsulfonyl)pyridin-2-yl][3-hydroxy-3-(trifluoromethyl)azetidin-1-yl]methanone;
{3-amino-5-[(3-fluorophenyl)sulfonyl]pyridin-2-yl}[3-hydroxy-3-(trifluoromethyl)azetidin-1-yl]methanone; and
3-amino-N-[(2S)-2-hydroxypropyl]-5-{[4-(trifluoromethoxy)phenyl]sulfonyl}pyridine-2-carboxamide.

Non-limiting examples of correctors include Lumacaftor (VX-809), 1-(2,2-difluoro-1,3-benzodioxol-5-yl)-N-{1-[(2R)-2,3-dihydroxypropyl]-6-fluoro-2-(1-hydroxy-2-methylpropan-2-yl)-1H-indol-5-yl}cyclopropanecarboxamide (VX-661), VX-983, GLPG2851, GLPG2222, GLPG2665, GLPG2737, GLPG3221, PTI-801, VX-152, VX-440, VX-445, VX-659, FDL169, FDL304, FD2052160, and FD2035659. Examples of correctors are also disclosed in Patent Application Publications WO2016069757, WO2016069891, WO2017009804, WO2017187321, WO2017060873, WO2017060874 and U.S. application Ser. Nos. 15/723,896 and 15/726,075.

In one embodiment, the corrector(s) can be selected from the group consisting of
Lumacaftor (VX-809);
1-(2,2-difluoro-1,3-benzodioxol-5-yl)-N-{1-[(2R)-2,3-dihydroxypropyl]-6-fluoro-2-(1-hydroxy-2-methylpropan-2-yl)-1H-indol-5-yl}cyclopropanecarboxamide (VX-661);
PTI-801;
VX-983;
GLPG2665;
GLPG2851;
GLPG2222;
GLPG2737;
GLPG3221;
VX-152;
VX-440;
VX-659;
VX-445;
FDL169
FDL304;
FD2052160;
FD2035659;
3-[(2R,4R)-4-({[1-(2,2-difluoro-1,3-benzodioxol-5-yl)cyclopropyl]carbonyl}amino)-7-methoxy-3,4-dihydro-2H-chromen-2-yl]benzoic acid;
3-[(2R,4R)-4-({[1-(2,2-difluoro-1,3-benzodioxol-5-yl)cyclopropyl]carbonyl}amino)-3,4-dihydro-2H-chromen-2-yl]benzoic acid;
3-[(2R,4R)-4-({[1-(2,2-difluoro-1,3-benzodioxol-5-yl)cyclopropyl]carbonyl}amino)-6-methyl-3,4-dihydro-2H-chromen-2-yl]benzoic acid;
3-[(2R,4R)-4-({[1-(2,2-difluoro-1,3-benzodioxol-5-yl)cyclopropyl]carbonyl}amino)-7-methyl-3,4-dihydro-2H-chromen-2-yl]benzoic acid;
3-[(2R,4R)-4-({[1-(2,2-difluoro-1,3-benzodioxol-5-yl)cyclopropyl]carbonyl}amino)-6-methoxy-3,4-dihydro-2H-chromen-2-yl]benzoic acid;
3-[(2R,4R)-4-({[1-(2,2-difluoro-1,3-benzodioxol-5-yl)cyclopropyl]carbonyl}amino)-7-(difluoromethoxy)-3,4-dihydro-2H-chromen-2-yl]cyclohexanecarboxylic acid;
3-[(2R,4R)-4-({[1-(2,2-difluoro-1,3-benzodioxol-5-yl)cyclopropyl]carbonyl}amino)-7-(difluoromethoxy)-3,4-dihydro-2H-chromen-2-yl]benzoic acid;
3-[(2R,4R)-4-({[1-(2,2-difluoro-1,3-benzodioxol-5-yl)cyclopropyl]carbonyl}amino)-7-methoxy-3,4-dihydro-2H-chromen-2-yl]cyclohexanecarboxylic acid;
3-[(2R,4R)-4-({[1-(2,2-difluoro-1,3-benzodioxol-5-yl)cyclopropyl]carbonyl}amino)-7-fluoro-3,4-dihydro-2H-chromen-2-yl]benzoic acid;
3-({3-[(2R,4R)-4-({[1-(2,2-difluoro-1,3-benzodioxol-5-yl)cyclopropyl]carbonyl}amino)-7-methyl-3,4-dihydro-2H-chromen-2-yl]benzoyl}amino)-1-methylcyclopentanecarboxylic acid;
3-[(2R,4R)-4-({[1-(2,2-difluoro-1,3-benzodioxol-5-yl)cyclopropyl]carbonyl}amino)-7-methyl-3,4-dihydro-2H-chromen-2-yl]-N-[(2R)-2,3-dihydroxypropyl]benzamide;
3-[(2R,4R)-4-({[1-(2,2-difluoro-1,3-benzodioxol-5-yl)cyclopropyl]carbonyl}amino)-7-(2-methoxyethoxy)-3,4-dihydro-2H-chromen-2-yl]benzoic acid;
3-[(2R,4R)-7-(benzyloxy)-4-({[1-(2,2-difluoro-1,3-benzodioxol-5-yl)cyclopropyl]carbonyl}amino)-3,4-dihydro-2H-chromen-2-yl]benzoic acid;
3-[(2R,4R)-4-({[1-(2,2-difluoro-1,3-benzodioxol-5-yl)cyclopropyl]carbonyl}amino)-7-(2-fluoroethoxy)-3,4-dihydro-2H-chromen-2-yl]benzoic acid;
3-[(2R,4R)-4-({[1-(2,2-difluoro-1,3-benzodioxol-5-yl)cyclopropyl]carbonyl}amino)-7-(trifluoromethyl)-3,4-dihydro-2H-chromen-2-yl]benzoic acid;
3-[(2R,4R)-4-({[1-(2,2-difluoro-1,3-benzodioxol-5-yl)cyclopropyl]carbonyl}amino)-7-(trifluoromethyl)-3,4-dihydro-2H-chromen-2-yl]cyclohexanecarboxylic acid;
4-[(2R,4R)-4-({[1-(2,2-difluoro-1,3-benzodioxol-5-yl)cyclopropyl]carbonyl}amino)-7-methoxy-3,4-dihydro-2H-chromen-2-yl]benzoic acid;
3-[(2R,4R)-4-({[1-(2,2-difluoro-1,3-benzodioxol-5-yl)cyclopropyl]carbonyl}amino)-8-fluoro-3,4-dihydro-2H-chromen-2-yl]benzoic acid;
4-[(2R,4R)-4-({[1-(2,2-difluoro-1,3-benzodioxol-5-yl)cyclopropyl]carbonyl}amino)-3,4-dihydro-2H-chromen-2-yl]benzoic acid;
4-[(2R,4R)-4-({[1-(2,2-difluoro-1,3-benzodioxol-5-yl)cyclopropyl]carbonyl}amino)-7-(difluoromethoxy)-3,4-dihydro-2H-chromen-2-yl]benzoic acid;
rac-3-[(2R,4S)-4-({[1-(2,2-difluoro-1,3-benzodioxol-5-yl)cyclopropyl]carbonyl}amino)tetrahydro-2H-pyran-2-yl]benzoic acid;
rac-4-[(2R,4S)-4-({[1-(2,2-difluoro-1,3-benzodioxol-5-yl)cyclopropyl]carbonyl}amino)tetrahydro-2H-pyran-2-yl]benzoic acid;
3-[(2S,4R)-4-({[1-(2,2-difluoro-1,3-benzodioxol-5-yl)cyclopropyl]carbonyl}amino)tetrahydro-2H-pyran-2-yl]benzoic acid;
3-[(2R,4S)-4-({[1-(2,2-difluoro-1,3-benzodioxol-5-yl)cyclopropyl]carbonyl}amino)tetrahydro-2H-pyran-2-yl]benzoic acid;
rac-3-[(2R,4S,6S)-4-({[1-(2,2-difluoro-1,3-benzodioxol-5-yl)cyclopropyl]carbonyl}amino)-6-phenyltetrahydro-2H-pyran-2-yl]benzoic acid;
3-[(2S,4R,6R)-4-({[1-(2,2-difluoro-1,3-benzodioxol-5-yl)cyclopropyl]carbonyl}amino)-6-phenyltetrahydro-2H-pyran-2-yl]benzoic acid;
3-[(2R,4S,6S)-4-({[1-(2,2-difluoro-1,3-benzodioxol-5-yl)cyclopropyl]carbonyl}amino)-6-phenyltetrahydro-2H-pyran-2-yl]benzoic acid;
4-[(2R,4S)-4-({[1-(2,2-difluoro-1,3-benzodioxol-5-yl)cyclopropyl]carbonyl}amino)tetrahydro-2H-pyran-2-yl]benzoic acid;
3-cyclobutyl-4-[4-(morpholin-4-yl)piperidin-1-yl]-1-phenyl-1H-pyrazolo[3,4-b]pyridine-6-carboxylic acid;
3-cyclobutyl-1-phenyl-4-{4-[(pyrrolidin-1-yl)methyl]piperidin-1-yl}-1H-pyrazolo [3,4-b]pyridine-6-carboxylic acid;
5-[(2R,4R)-4-{[(7R)-2,2-difluoro-7-methyl-6,7-dihydro-2H-furo[2,3-][1,3]benzodioxole-7-carbonyl]amino}-7-methoxy-3,4-dihydro-2H-1-benzopyran-2-yl]pyrazine-2-carboxylic acid;
6-[(2R,4R)-4-{[(7R)-2,2-difluoro-7-methyl-6,7-dihydro-2H-furo[2,3-][1,3]benzodioxole-7-carbonyl]amino}-7-(trifluoromethoxy)-3,4-dihydro-2H-1-benzopyran-2-yl]pyridine-3-carboxylic acid;
trans-4-[(2S,4S)-4-{[(7R)-2,2-difluoro-7-methyl-6,7-dihydro-2H-furo[2,3-][1,3]benzodioxole-7-carbonyl]amino}-7-(trifluoromethoxy)-3,4-dihydro-2H-1-benzopyran-2-yl]cyclohexane-1-carboxylic acid;
6-[(2R,4R)-7-(difluoromethoxy)-4-{[(7R)-2,2-difluoro-7-methyl-6,7-dihydro-2H-furo[2,3-f][1,3]benzodioxole-7-carbonyl]amino}-3,4-dihydro-2H-1-benzopyran-2-yl]pyridine-3-carboxylic acid;
trans-4-[(2S,4S)-4-{[(7R)-2,2-difluoro-7-methyl-6,7-dihydro-2H-furo[2,3-f][1,3]benzodioxole-7-carbonyl]amino}-7-methoxy-3,4-dihydro-2H-1-benzopyran-2-yl]cyclohexane-1-carboxylic acid;
ethyl trans-4-[(2S,4S)-7-(difluoromethoxy)-4-{[(7R)-2,2-difluoro-7-methyl-6,7-dihydro-2H-furo[2,3-f][1,3]benzodioxole-7-carbonyl]amino}-3,4-dihydro-2H-1-benzopyran-2-yl]cyclohexane-1-carboxylate;
cis-4-[(2R,4R)-4-{[(7R)-2,2-difluoro-7-methyl-6,7-dihydro-2H-furo[2,3-][1,3]benzodioxole-7-carbonyl]amino}-7-(trifluoromethoxy)-3,4-dihydro-2H-1-benzopyran-2-yl]cyclohexane-1-carboxylic acid;
trans-4-[(2S,4S)-7-(difluoromethoxy)-4-{[(7R)-2,2-difluoro-7-methyl-6,7-dihydro-2H-furo[2,3-][1,3]benzodioxole-7-carbonyl]amino}-3,4-dihydro-2H-1-benzopyran-2-yl]cyclohexane-1-carboxylic acid;
1-[(2R,4R)-4-{[(7R)-2,2-difluoro-7-methyl-6,7-dihydro-2H-furo[2,3-][1,3]benzodioxole-7-carbonyl]amino}-7-(trifluoromethoxy)-3,4-dihydro-2H-1-benzopyran-2-yl]cyclopropane-1-carboxylic acid;
trans-4-[(2R,4R)-4-{[(5S)-2,2-difluoro-5-methyl-6,7-dihydro-2H,5H-indeno [5,6-d][1,3]dioxole-5-carbonyl]amino}-7-(trifluoromethoxy)-3,4-dihydro-2H-1-benzopyran-2-yl]cyclohexane-1-carboxylic acid;
trans-4-[(2R,4R)-4-{[(5S)-2,2-difluoro-5-methyl-6,7-dihydro-2H,5H-indeno [5,6-d][1,3]dioxole-5-carbonyl]amino}-7-methoxy-3,4-dihydro-2H-1-benzopyran-2-yl]cyclohexane-1-carboxylic acid;
trans-4-[(2R,4R)-4-{[(7R)-2,2-difluoro-7-methyl-6,7-dihydro-2H-furo[2,3-][1,3]benzodioxole-7-carbonyl]amino}-7-methoxy-3,4-dihydro-2H-1-benzopyran-2-yl]cyclohexane-1-carboxylic acid;
trans-4-[(2R,4R)-7-(difluoromethoxy)-4-{[(7R)-2,2-difluoro-7-methyl-6,7-dihydro-2H-furo[2,3-f][1,3]benzodioxole-7-carbonyl]amino}-3,4-dihydro-2H-1-benzopyran-2-yl]cyclohexane-1-carboxylic acid; and
trans-4-[(2R,4R)-4-{[(7R)-2,2-difluoro-7-methyl-6,7-dihydro-2H-furo[2,3-][1,3]benzodioxole-7-carbonyl]amino}-7-(trifluoromethoxy)-3,4-dihydro-2H-1-benzopyran-2-yl]cyclohexane-1-carboxylic acid.

In one embodiment, the additional therapeutic agent is a CFTR amplifier. CFTR amplifiers enhance the effect of known CFTR modulators, such as potentiators and correctors. Examples of CFTR amplifiers are PTI130 and PTI-428. Examples of amplifiers are also disclosed in publications: WO2015138909 and WO2015138934.

In one embodiment, the additional therapeutic agent is a CFTR stabilizer. CFTR stabilizers enhance the stability of corrected CFTR that has been treated with a corrector, corrector/potentiator or other CFTR modulator combination(s). An example of a CFTR stabilizer is cavosonstat (N91115). Examples of stabilizers are also disclosed in publication: WO2012048181.

In one embodiment, the additional therapeutic agent is an agent that reduces the activity of the epithelial sodium channel blocker (ENaC) either directly by blocking the channel or indirectly by modulation of proteases that lead to an increase in ENaC activity (e.g., serine proteases, channel-activating proteases). Exemplary of such agents include camostat (a trypsin-like protease inhibitor), QAU145, 552-02, GS-9411, INO-4995, Aerolytic, amiloride, and VX-371. Additional agents that reduce the activity of the epithelial sodium channel blocker (ENaC) can be found, for example, in PCT Publication No. WO2009074575 and WO2013043720; and U.S. Pat. No. 8,999,976.

In one embodiment, the ENaC inhibitor is VX-371.

In one embodiment, the ENaC inhibitor is SPX-101 (S18).

In one embodiment, the present invention provides pharmaceutical compositions comprising a compound of the invention, or a pharmaceutically acceptable salt thereof, and one or more additional therapeutic agents. In a particular embodiment, the additional therapeutic agents are selected from the group consisting of CFTR modulators and CFTR amplifiers. In a further embodiment, the additional therapeutic agents are CFTR modulators. In one embodiment, the present invention provides pharmaceutical compositions comprising a compound of the invention, or a pharmaceutically acceptable salt thereof, one potentiator, and one or more additional correctors.

This invention also is directed to kits that comprise one or more compounds and/or salts of the invention, and, optionally, one or more additional therapeutic agents.

This invention also is directed to methods of use of the compounds, salts, compositions, and/or kits of the invention to, with or without one or more additional therapeutic agents, for example, modulate the Cystic Fibrosis Transmembrane Conductance Regulator (CFTR) protein, and treat a disease treatable by modulating the Cystic Fibrosis Transmembrane Conductance Regulator (CFTR) protein (including cystic fibrosis, Sjögren's syndrome, pancreatic insufficiency, chronic obstructive lung disease, and chronic obstructive airway disease).

### Chemical Synthetic Procedures

### General

The compounds of the invention can be prepared from readily available starting materials using the following general methods and procedures. It will be appreciated that where typical or preferred process conditions (i.e. reaction temperatures, times, mole ratios of reactants, solvents, pressures, etc.) were given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art (Protective Groups in Organic Synthesis Third Edition; Greene, T W and Wuts, P G M, Eds.; Wiley-Interscience: New York, 1991).

The following methods are presented with details as to the preparation of a compound of the invention as defined hereinabove and the comparative examples. A compound of the invention may be prepared from known or commercially available starting materials and reagents by one skilled in the art of organic synthesis.

All reagents were of commercial grade and were used as received without further purification, unless otherwise stated. Commercially available anhydrous solvents were used for reactions conducted under inert atmosphere. Reagent grade solvents were used in all other cases, unless otherwise specified. Column chromatography was performed on silica gel 60 (35-70 μm). Thin layer chromatography was carried out using pre-coated silica gel F-254 plates (thickness 0.25 mm). ¹H NMR spectra were recorded on a Bruker Advance 300 NMR spectrometer (300 MHz), an Agilent 400 MHz NMR spectrometer or a 500 MHz spectrometer. Chemical shifts (δ ppm) for ¹H NMR spectra were reported in parts per million (ppm) relative to tetramethylsilane (δ ppm 0.00) or the appropriate residual solvent peak, i.e. CHCl₃ (δ ppm 7.27), as internal reference. Multiplicities were given as singlet (s), doublet (d), doublet of doublets of doublets (ddd), doublet of doublets of doublets of doublets (dddd), doublet of doublets of quartets (ddq), doublet of doublets of triplets (ddt), doublet of quartets (dq), doublet of triplets of doublets (dtd), heptet (hept), triplet (t), triplet of doublets of doublets (tdd), triplet of quartets (tq), quartet (q), quartet of doublets (qd), quartet of triplets (qt), quintuplet (quin), multiplet (m) and broad (br). Electrospray MS spectra were obtained on a Waters platform LC/MS spectrometer or with Waters Acquity H-Class UPLC coupled to a Waters Mass detector 3100 spectrometer or with Agilent 6130 Quadrupole LC/MS. Columns used: Waters Acquity UPLC BEH C18 1.7 μm, 2.1 mm ID×50 mm L, Waters Acquity UPLC BEH C18, 1.7 μm, 2.1 mm ID×30 mm L, or Waters Xterra® MS 5 μm C18, 100×4.6 mm. Ascetis Express C-18, 2.7 μm, 4.6 mm ID×10 cm L. The methods were using either CH₃CN/H₂O gradients (H₂O contains either 0.1% TFA or 0.1% NH₃ or 0.1% HCO₂H) or CH₃OH/H₂O gradients (H₂O contains 0.05% TFA). Microwave heating was performed with a Biotage® Initiator.

Racemic mixtures were separated on an Agilent HP1100 system with UV detection. Column used: Chiralpak® IA (10×250 mm, 5 μm). Solvents used: iPrOH and tBME. Enantiomeric purity was determined on an Agilent HP1100 system with UV detection. Column used: Chiralpak® IA (4.6×250 mm, 5 μm). Solvents used: iPrOH and tBME.

Stereochemistry of final compounds was arbitrarily assigned in some cases, based on the order of elution and/or activity with respect to existing analogs.

List of abbreviations used in the experimental section:

| Abbreviation | Definition |
|---|---|
| MeCN | acetonitrile |
| TFA | trifluoroacetic acid |
| NMR | nuclear magnetic resonance |
| DMSO | dimethyl sulfoxide |
| LC/MS or | liquid chromatography- mass spectrometry |
| LCMS | |
| tBME | tert-butyl methyl ether |
| s | singlet |
| br s | broad singlet |
| d | duplet or doublet |
| dd | double duplet or doublet of doublets |
| m | multiplet |
| min | minute |
| mL or ml | milliliter |
| μL | microliter |
| g | gram |
| mg | milligram |
| mmol | millimoles |
| HPLC | high pressure liquid chromatography |
| Ppm | parts per million |
| iPrOH | iso-propanol |
| HATU | 1-[bis(dimethylamino)methylene]-1H-1,2,3-triazolo[4,5- |
| | b]pyridinium 3-oxid hexafluorophosphate |
| psig | pounds per square inch gauge |
| PE | polyethylene |

### Synthetic Preparation of the Compounds of the Invention

### Schemes

The compounds of the present disclosure can be better understood in connection with the following synthetic schemes and methods which illustrate a means by which the compounds can be prepared. The compounds of this disclosure can be prepared by a variety of synthetic procedures. Representative procedures are shown in, but are not limited to, Schemes 1-4.

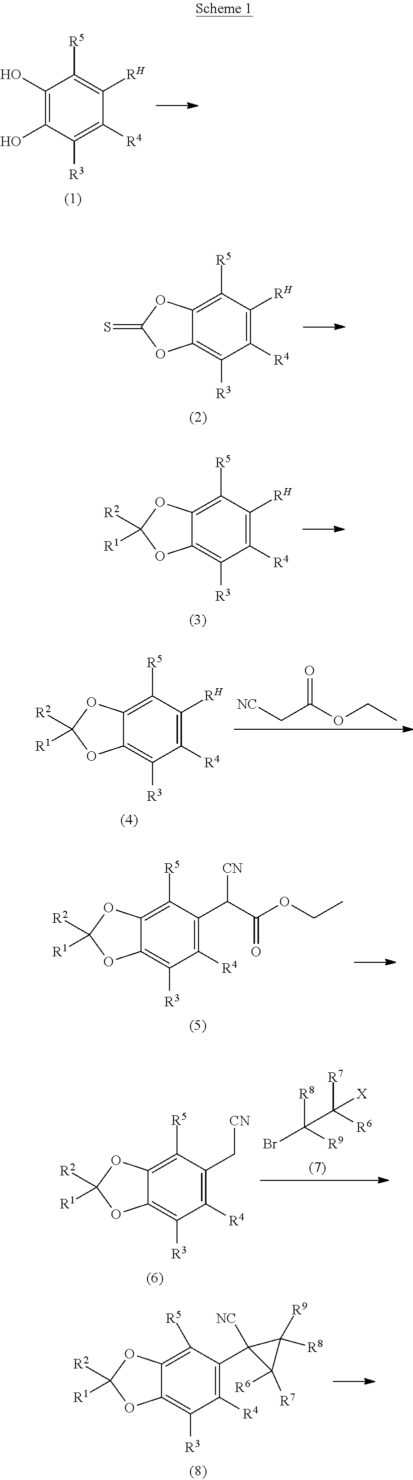

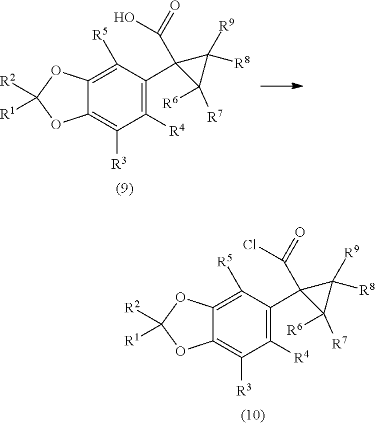

As shown in Scheme 1, compounds of Formula (10) can be prepared from compounds of Formula (1). Compounds of Formula (1), wherein R³, R⁴, R⁵, and R^{H} are each independently hydrogen or deuterium, can be treated with 1, 1-thiocarbonylimidazole to provide compounds of Formula (2). The reaction is typically performed at ambient temperature in a solvent such as, but not limited to, tetrahydrofuran. Compounds of Formula (2) can be treated with n-Bu₄NH₂F₃ and N-iodosuccinimide at low temperature before warming up to ambient temperature to provide compounds of Formula (3) wherein R¹ and R² are fluoro. Alternatively, compounds of Formula (2) can be treated with Raney®-nickel in refluxing benzene to provide compounds of Formula (3) wherein R¹ and R² are hydrogen. Compounds of Formula (3) can be treated with bromine in the presence of hydrogen fluoride and a Friedel-Crafts catalyst such as, but not limited to titanium tetrachloride, to provide compounds of Formula (4). The addition is typically performed at low temperature before warming up to ambient temperature. Compounds of Formula (5) can be prepared by treating compounds of Formula (4) with a mixture of ethyl 2-cyanoacetate, trisodium phosphate, tributyl phosphine, and a catalyst such as but not limited to tris(dibenzylideneacetone)dipalladium(0). The reaction is typically performed at an elevated temperature under nitrogen in a solvent such as, but not limited to, toluene. Compounds of Formula (5) can be treated with hydrochloric acid to provide compounds of Formula (6). The reaction is typically performed at an elevated temperature in a solvent such as, but not limited to, dimethyl sulfoxide. Compounds of Formula (6) can be treated with tetrabutylammonium bromide, a base such as but not limited to sodium hydroxide, and compounds of Formula (7) wherein R⁶, R⁷, R⁸, and R⁹ are each independently hydrogen or deuterium, and X is Cl or Br, to provide compounds of Formula (8). Compounds of Formula (9) can be prepared by treating compounds of Formula (8) with sodium hydroxide. The reaction is typically performed at an elevated temperature in a solvent such as but not limited to ethanol, water, or mixtures thereof. Compounds of Formula (9) can be treated with thionyl chloride to provide compounds of Formula (10). The reaction is typically performed at an elevated temperature in a solvent such as but not limited to toluene, N,N-dimethylformamide, dichloromethane, or mixtures thereof. Alternatively, compounds of Formula (9) can be treated with oxalyl chloride with a catalytic amount of N,N-dimethylformamide to provide compounds of Formula (10). The reaction is typically performed at an elevated temperature in a solvent such as but not limited to toluene, dichloromethane, or mixtures thereof.

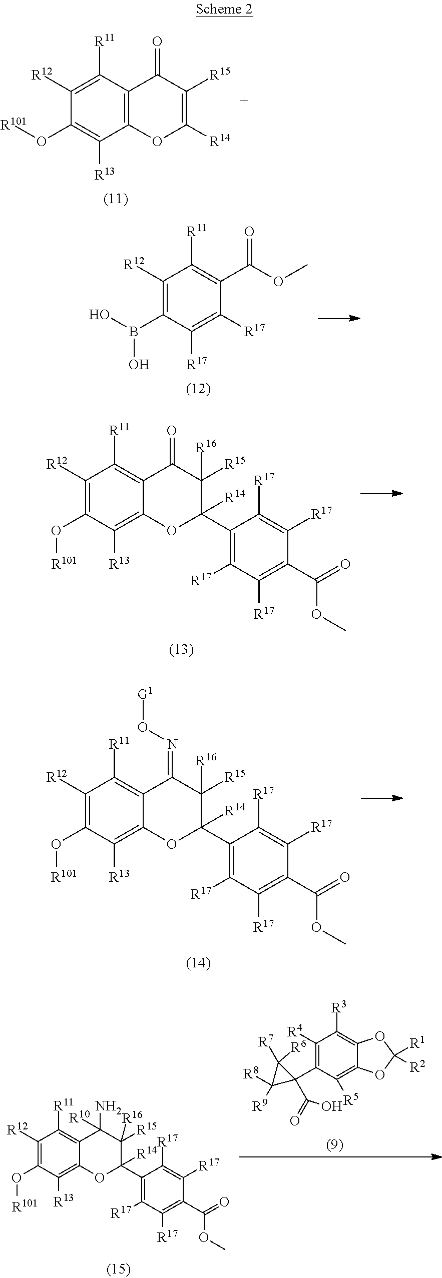

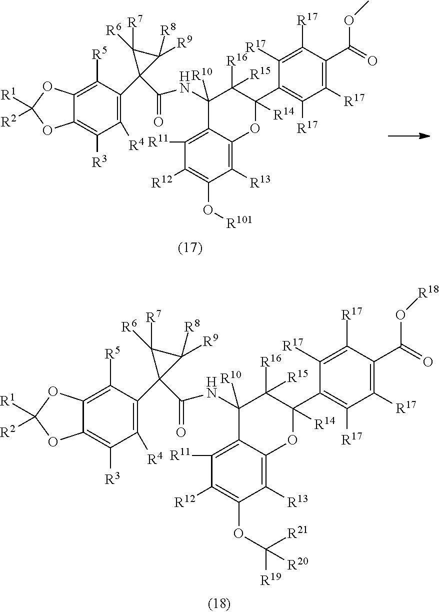

As shown in Scheme 2, treatment of compounds of Formula (11), wherein R¹¹, R¹², R¹³, R¹⁴, and R¹⁵ are hydrogen or deuterium and R¹⁰¹ is hydrogen, methyl, or an appropriate protecting group, with a boronic acid (or ester thereof) of Formula (12) wherein each R¹⁷ is independently hydrogen or deuterium, in the presence of (S)-4-(tert-butyl)-2-(pyridin-2-yl)-4,5-dihydrooxazole, and a catalyst such as bis(2,2,2-trifluroacetoxy)palladium will provide chromanones of Formula (13). Chromanones (13) may be treated with hydroxylamines or alkoxyamines such as methoxyamine or benzylhydroxylamine to provide oximes of Formula (14), wherein GI is methyl or benzyl. The oxime group of (14) may be reduced using methodologies known by one skilled in the art, for example, by hydrogenolysis in the presence of hydrogen and a catalyst such as, but not limited to, platinum on carbon, or Raney®-nickel, or platinum (IV) oxide, to provide the amines of Formula (15). Acids of Formula (9), which can be prepared as described in Scheme 1, may be reacted with amines of Formula (15) in the presence of 1-[bis(dimethylamino)methylene]-1H-1,2,3-triazolo[4,5-b]pyridinium 3-oxid hexafluorophosphate, and a base such as, but not limited to, diisopropyl ethyl amine, in a solvent such as, but not limited to, N,N-dimethylformamide, at ambient temperature to provide amides of Formula (17). Compounds of Formula (17) can be deprotected if necessary, and then treated with diethyl (bromodifluoromethyl)phosphonate in the presence of a base such as but not limited to potassium hydroxide, to provide compounds of Formula (18) wherein R¹⁹ and R²⁰ are fluoro and R²¹ is hydrogen. The reaction is typically performed in a solvent such as but not limited to acetonitrile, water, and mixtures thereof.

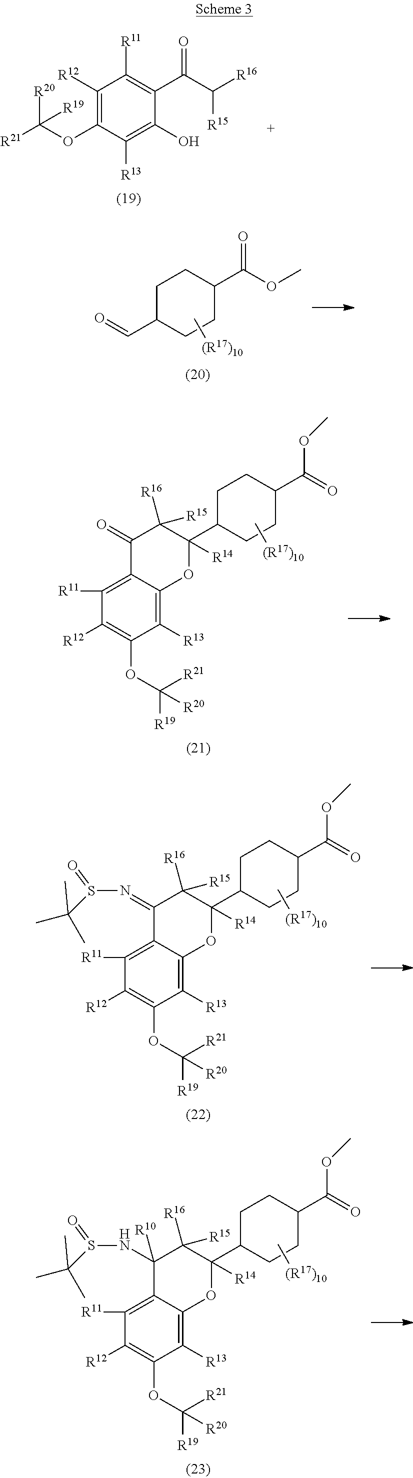

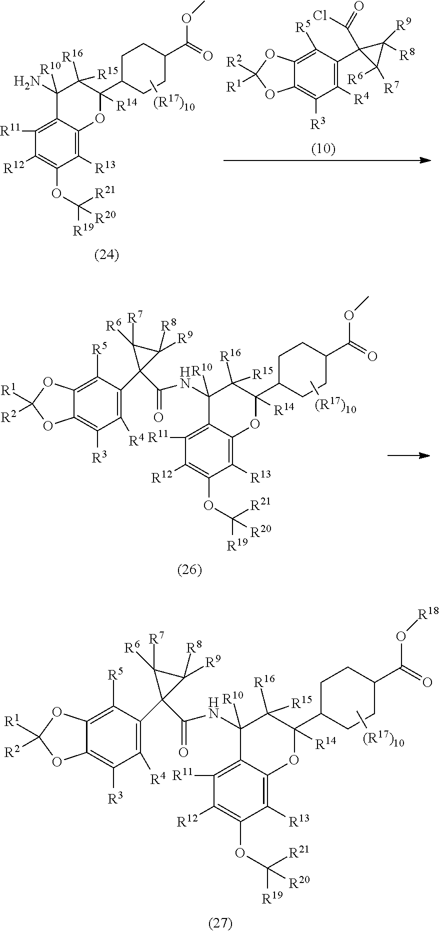

Compounds of Formula (19), wherein R¹¹, R¹², R¹³, R¹⁵, R¹⁶, are hydrogen or deuterium, and R¹⁹, R²⁰, and R²¹ are each independently hydrogen, deuterium, or fluorine, may be reacted with aldehydes of Formula (20), wherein each R¹⁷ is independently hydrogen or deuterium, in the presence of pyrrolidine and optionally, acetic acid, to provide compounds of Formula (21). The reaction is typically performed at an elevated temperature, for example, at about 70° C., and in a solvent, such as, but not limited to, methanol or toluene. The hydrochloride salt of amine (24) may be prepared from ketones of Formula (21) according to the general procedure described by Ellman and co-workers (Tanuwidjaja, J.; Ellman, J. A. et al. J Org. Chem. 2007, 72, 626) as illustrated in Scheme 3. Chromanones (21) may be condensed with a chiral sulfinamide such as tert-butanesulfinamide in the presence of a Lewis acid such as titanium(IV) ethoxide to provide N-sulfinyl imine intermediate (22). N-Sulfinyl imine intermediate (22) can alternatively be prepared as described in Scheme 4. Diastereomeric mixtures of (22) may optionally be separated via chromatography, and may undergo a subsequent reduction with reagents such as sodium borohydride to provide sulfinamides of general Formula (23). Treatment of the sulfinamides (23) with HCl or acetyl chloride/methanol will provide the hydrochloride salts of amine (24). Reaction of acid chlorides of Formula (10), which can be prepared as described in Scheme 1, with amines of Formula (24) optionally in the presence of a base such as, but not limited to, a tertiary amine base (for example, triethylamine or N,N-diisopropylethylamine) or an aromatic base such as pyridine, at room temperature or heated in a solvent such as dichloromethane will provide amides of Formula (26). Compounds of Formula (26) can be treated with sodium hydroxide to provide compounds of Formula (27), which are representative of compounds of the invention. The reaction is typically performed at an elevated temperature in a solvent such as but not limited to tetrahydrofuran, methanol, or mixtures thereof.

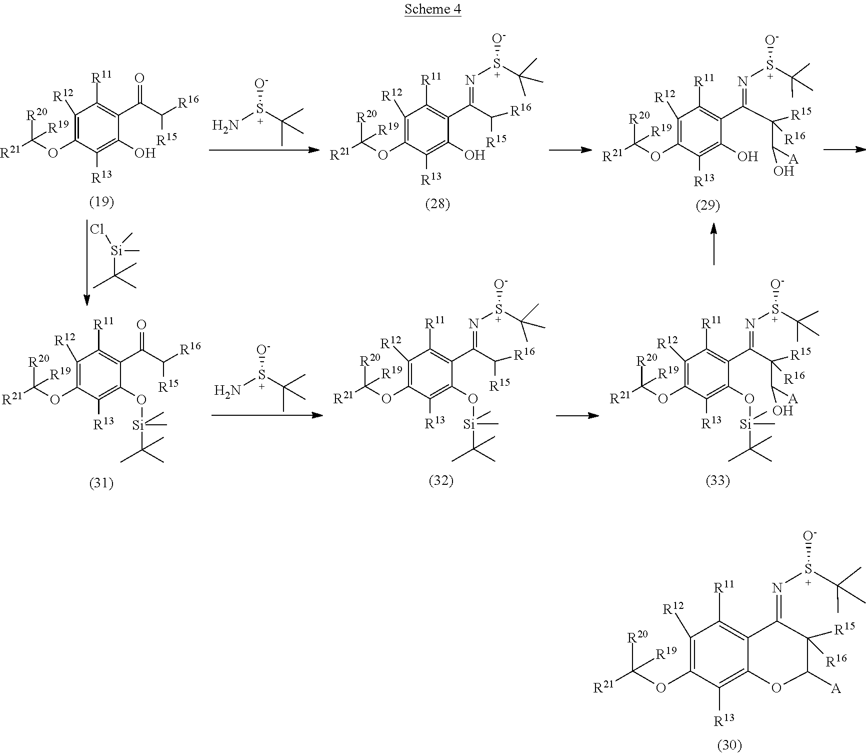

Scheme 4 illustrates an alternative route for the preparation of representative N-sulfinyl imine intermediates of Formula (30).

Compounds of Formula (19) may be treated with a chiral sulfinamide such as tert-butanesulfinamide in the presence of a Lewis acid such as titanium(IV) ethoxide to provide N-sulfinyl imine intermediates (28). Compounds of Formula (28) may be treated with aldehydes of formula A-CHO in the presence of lithium diisopropanamide (prepared in situ from n-butyllithium and N,N-diisopropylamine) to provide compounds of Formula (29). The diastereomeric mixture of Formula (29) may be separated via chromatography. Treatment of (29) with diethyl azodicarboxylate in the presence of triphenylphosphine provides N-sulfinyl imine intermediate of Formula (30).

Alternatively, the hydroxyl functionality of compounds of Formula (19) may be protected before treatment with the chiral sulfinamide. For example, compounds of Formula (19) may be treated with tert-butyldimethylsilyl chloride in the presence of an organic base such as, but not limited to, triethylamine to provide compounds of Formula (31). Treatment of (31) with a chiral sulfinamide such as tert-butanesulfinamide in the presence of a Lewis acid such as titanium(IV) ethoxide provides the intermediate (32). Reaction of (32) with aldehydes of formula A-CHO in the presence of lithium diisopropylamide (prepared in situ from n-butyllithium and N,N-diisopropylamine) provides compounds of Formula (33). The diastereomeric mixture of Formula (33) may be separated via chromatography. Subsequent removal of the silyl protecting group provides compounds of Formula (29).

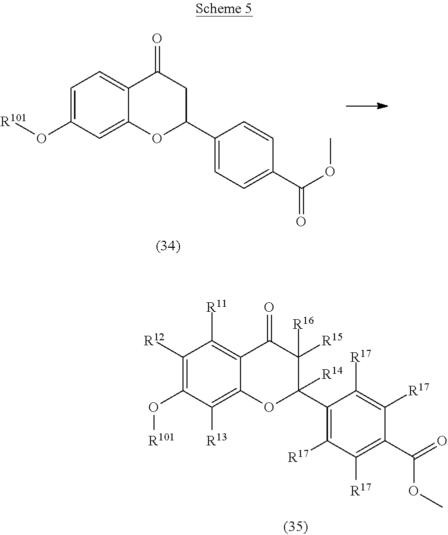

As shown in Scheme 5, compounds of formula (34) can be treated with D₂O in the presence of Pt/C, to provide compounds of formula (35). The reaction is typically performed at an elevated temperature, under an argon pressurized atmosphere, in a solvent such as, but not limited to, isopropanol, cyclohexane, or mixtures thereof.

### Synthetic Procedures

### Example 1

### 4-[(2R,4R)-4-({[1-(2,2-difluoro-1,3-benzodioxol-5-yl)cyclopropyl]carbonyl}amino)-7-(difluoromethoxy)(3,3,4,5,6-²H₅)-3,4-dihydro-2H-chromen-2-yl](3,5-²H₂)benzoic acid

### Example 1A

### methyl 4-[(2R)-7-methoxy-4-oxo-3,4-dihydro-2H-chromen-2-yl]benzoate

A 20 mL vial was charged with bis(2,2,2-trifluoroacetoxy)palladium (0.264 g, 0.795 mmol), (S)-4-(tert-butyl)-2-(pyridin-2-yl)-4,5-dihydrooxazole (0.195 g, 0.954 mmol), ammonium hexafluorophosphate(V) (0.777 g, 4.77 mmol) and (4-(methoxycarbonyl)phenyl)boronic acid (2.86 g, 15.89 mmol). The reaction was stirred in dichloroethane (5 mL) for 5 minutes, and a pale brown colored suspension was observed. To the suspension was added 7-methoxy-4H-chromen-4-one (1.4 g, 7.95 mmol) and water (0.716 mL, 39.7 mmol). The sides of the vial were washed with more dichloroethane (5 mL). The vial was capped and the mixture was stirred at 60° C. overnight. The mixture was filtered through a plug of silica gel and diatomaceous earth and eluted with ethyl acetate to give a red solution. The solvent was removed under reduced pressure and the crude material was chromatographed using a 24 g silica gel cartridge with a gradient of 5-60% ethyl acetate/heptanes over 20 minutes. The white solid was collected by filtration and the filtrate was concentrated. The residue was chromatographed using a 12 g cartridge eluting with 100% dichloromethane to give a white solid which was combined with the solid collected by filtration to give the title compound (1.6 g, 5.12 mmol, 64.5% yield) as a white solid. ¹H NMR (400 MHz, DMSO-d₆) δ ppm 8.02 (dd, J=8.4, 2.1 Hz, 2H), 7.75-7.72 (m, 1H), 7.70 (d, J=8.4 Hz, 2H), 6.72-6.66 (m, 2H), 5.77 (dd, J=12.9, 3.1 Hz, 1H), 3.87 (s, 3H), 3.83 (d, J=2.0 Hz, 3H), 3.14 (dd, J=16.8, 12.9 Hz, 1H), 2.82 (dd, J=16.7, 3.1 Hz, 1H). MS (ESI+) m/z 313 (M+H)⁺.

### Example 1B

### methyl 4-[(2R)-7-methoxy-4-oxo(3,3,5,6-²H₄)-3,4-dihydro-2H-chromen-2-yl](2,3,5-²H₃)benzoate

The H/D-exchange was performed with 6 g total of chromanone in 2 batches. Methyl 4-[(2R)-7-methoxy-4-oxo-3,4-dihydro-2H-chromen-2-yl]benzoate (3.4 g, 10.89 mmol) and 10% of Pt/C (1.7 g) were added to a Parr reactor. To the reactor were added deuterium oxide (262 mL), isopropanol (13 mL) and cyclohexane (118 mL). The second batch was performed using methyl 4-[(2R)-7-methoxy-4-oxo-3,4-dihydro-2H-chromen-2-yl]benzoate (2.6 g, 8.32 mmol), 10% of Pt/C (1.3 g), deuterium oxide (200 mL), isopropanol (10 mL) and cyclohexane (90 mL). Each Parr reactor was sealed, pressurized to 100 psig under argon, heated to 100° C., and stirred for 16 hours. The two crude reaction mixtures were combined and diluted with dichloromethane (400 mL). The biphasic mixture was mixed for 15 minutes and filtered to remove the solids. The filter cake was rinsed with dichloromethane (100 mL), and the combined filtrates were transferred to a separatory funnel. The lower organic layer was removed, and the aqueous layer was washed with dichloromethane (150 mL). The combined dichloromethane solution was concentrated to dryness in vacuo to furnish an off-white solid. The crude product was purified by column chromatography on an Isco chromatography system (120-g column; gradient: 2 column volume heptanes, ramp up to 70:30 heptanes/ethyl acetate over 7 column volume, hold at 70:30 for 6 column volume) to provide 3.6 g of the title compound in 86% ee. The white solid was slurried in a 5:3 mix of ethyl acetate/heptanes (30 mL) for 26 hours. The slurry was filtered to isolate the product, which was dried in a vacuum oven for 19 hours at 40° C. to provide 2.92 g (49% yield) of the title compound in 93.5% ee. ¹H NMR (400 MHz, CDCl₃) δ ppm 8.13-8.10 (m, 1.3H), 7.88 (s, 0.1H), 7.57-7.55 (m, 0.3H), 6.53 (s, 1H), 5.56-5.53 (m, 1H), 3.94 (s, 3H), 3.86 (s, 3H), 3.03-2.84 (m, 0.4H).

Mass fragment data for native and labeled:

Native Substrate

| m/z | abund |
|---|---|
| 313 | 100.00% |
| 314 | 19.10% |
| 315 | 3.20% |
| 316 | 0.20% |
| 317 | 0.00% |
| 315 | 0.00% |
| 319 | 0.00% |
| 320 | 0.00% |
| 321 | 0.00% |
| 322 | 0.00% |
| 323 | 0.00% |

Obs Sample

| | | normalized | normalized | |
|---|---|---|---|---|
| m/z | obs abund | obs | calc | (obs-calc) |
| 313 | 3.10% | 3.10% | 3.10% | 0.00% |
| 314 | 11.00% | 11.00% | 11.00% | 0.00% |
| 315 | 21.60% | 21.60% | 21.60% | 0.00% |
| 316 | 39.10% | 39.10% | 39.10% | 0.00% |
| 317 | 61.10% | 61.10% | 61.10% | 0.00% |
| 318 | 88.10% | 88.10% | 88.10% | 0.00% |
| 319 | 100.00% | 100.00% | 100.00% | 0.00% |
| 320 | 82.90% | 82.90% | 82.90% | 0.00% |
| 321 | 51.70% | 51.70% | 51.70% | 0.00% |
| 322 | 25.80% | 25.80% | 25.80% | 0.00% |
| 323 | 11.40% | 11.40% | 11.40% | 0.00% |
| 324 | 4.30% | 4.30% | 4.30% | 0.00% |
| 325 | 1.30% | 1.30% | 1.30% | 0.00% |
| 326 | 0.50% | 0.50% | 0.48% | 0.02% |
| 327 | 0.00% | 0.00% | 0.08% | −0.08% |
| 328 | 0.00% | 0.00% | 0.01% | −0.01% |
| 329 | 0.00% | 0.00% | 0.00% | 0.00% |
| 330 | 0.00% | 0.00% | 0.00% | 0.00% |
| 331 | 0.00% | 0.00% | 0.00% | 0.00% |
| 332 | 0.00% | 0.00% | 0.00% | 0.00% |
| 333 | 0.00% | 0.00% | 0.00% | 0.00% |
| 334 | 0.00% | 0.00% | 0.00% | 0.00% |
| 335 | 0.00% | 0.00% | 0.00% | 0.00% |
| 336 | 0.00% | 0.00% | 0.00% | 0.00% |
| 337 | 0.00% | 0.00% | 0.00% | 0.00% |
| 338 | 0.00% | 0.00% | 0.00% | 0.00% |
| 339 | 0.00% | 0.00% | 0.00% | 0.00% |
| 340 | 0.00% | 0.00% | 0.00% | 0.00% |
| 341 | 0.00% | 0.00% | 0.00% | 0.00% |
| 342 | 0.00% | 0.00% | 0.00% | 0.00% |
| 343 | 0.00% | 0.00% | 0.00% | 0.00% |
| 344 | 0.00% | 0.00% | 0.00% | 0.00% |

### Example 1C

### methyl 4-[(2R,4E)-4-[(benzyloxy)imino]-7-methoxy(3,3,5,6-²H₄)-3,4-dihydro-2H-chromen-2-yl](2,3,5-²H₃)benzoate

Methyl 4-[(2R)-7-methoxy-4-oxo(3,3,5,6-²H₄)-3,4-dihydro-2H-chromen-2-yl](2,3,5-²H₃)benzoate (2.9 g, 9.29 mmol) was added to a 100-mL round-bottom flask and pyridine (20 ml) was added. The mixture was stirred until the yellow solids were dissolved, then O-benzylhydroxylamine hydrochloride (1.586 g, 9.94 mmol) was added. The mixture was warmed to 50° C. and mixed for 18 hours. The reaction was cooled to room temperature, and the pyridine was removed in vacuo to furnish a white slurry. The slurry was diluted with ethyl acetate (35 mL) and 10 wt % aqueous NH₄Cl (15 mL). The solution was transferred to a separatory funnel and the aqueous layer was removed. The organic phase was washed with 1M aqueous HCl (2×30 mL), and 5% aqueous NaCl (15 mL). The organic solution was transferred to a 100-mL round bottom flask and concentrated in vacuo to furnish an off-white foam. To the foam was added ethyl acetate (3.8 mL) and heptanes (45 mL). The resulting mixture was sonicated for 15 minutes, which led to the precipitation of a white solid. The mixing was continued for another 45 minutes, and the precipitate was collected by filtration. The wet cake was rinsed with heptanes (5 mL). The solids were dried in a vacuum oven at 60° C. for 3.5 hours to provide 3.23 g (83% yield) of methyl 4-[(2R,4E)-4-[(benzyloxy)imino]-7-methoxy(3,3,5,6-²H₄)-3,4-dihydro-2H-chromen-2-yl](2,3,5-²H₃)benzoate. The isolated solid showed incorporation of 6.7 D-atoms by APCI-LC/MS. ¹H NMR (400 MHz, CDCl₃) δ ppm 8.11-8.08 (m, 1.5H), 7.88 (m, 0.1H), 7.56-7.54 (m, 0.4H), 7.45-7.31 (m, 6H), 6.57 (s, 0.1H), 6.52 (s, 0.9H), 5.33-5.13 (m, 3.5H), 3.96 (s, 3H), 3.82 (s, 3H), 2.88-2.67 (m, 0.3H).

Mass fragment data for native and labeled:

Native Substrate

| m/z | abund |
|---|---|
| 418 | 100.00% |
| 419 | 27.80% |
| 420 | 0.00% |
| 421 | 0.00% |
| 422 | 0.00% |
| 423 | 0.00% |
| 424 | 0.00% |
| 425 | 0.00% |
| 426 | 0.00% |
| 427 | 0.00% |
| 428 | 0.00% |

Obs Sample

| | | normalized | normalized | |
|---|---|---|---|---|
| m/z | obs abund | obs | calc | (obs-calc) |
| 418 | 0.00% | 0.00% | 0.00% | 0.00% |
| 419 | 0.00% | 0.00% | 0.00% | 0.00% |
| 420 | 0.00% | 0.00% | 0.00% | 0.00% |
| 421 | 0.00% | 0.00% | 0.00% | 0.00% |
| 422 | 18.90% | 18.90% | 18.90% | 0.00% |
| 423 | 56.50% | 56.50% | 56.50% | 0.00% |
| 424 | 93.20% | 93.20% | 93.20% | 0.00% |
| 425 | 100.00% | 100.00% | 100.00% | 0.00% |
| 426 | 71.00% | 71.00% | 71.00% | 0.00% |
| 427 | 36.10% | 36.10% | 36.09% | 0.01% |
| 428 | 17.30% | 17.30% | 17.34% | −0.04% |
| 429 | 5.10% | 5.10% | 4.96% | 0.14% |
| 430 | 0.00% | 0.00% | 0.52% | −0.52% |
| 431 | 0.00% | 0.00% | 0.00% | 0.00% |
| 432 | 0.00% | 0.00% | 0.00% | 0.00% |
| 433 | 0.00% | 0.00% | 0.00% | 0.00% |
| 434 | 0.00% | 0.00% | 0.00% | 0.00% |
| 435 | 0.00% | 0.00% | 0.00% | 0.00% |
| 436 | 0.00% | 0.00% | 0.00% | 0.00% |
| 437 | 0.00% | 0.00% | 0.00% | 0.00% |
| 438 | 0.00% | 0.00% | 0.00% | 0.00% |
| 439 | 0.00% | 0.00% | 0.00% | 0.00% |
| 440 | 0.00% | 0.00% | 0.00% | 0.00% |
| 441 | 0.00% | 0.00% | 0.00% | 0.00% |
| 442 | 0.00% | 0.00% | 0.00% | 0.00% |
| 443 | 0.00% | 0.00% | 0.00% | 0.00% |
| 444 | 0.00% | 0.00% | 0.00% | 0.00% |
| 445 | 0.00% | 0.00% | 0.00% | 0.00% |
| 446 | 0.00% | 0.00% | 0.00% | 0.00% |
| 447 | 0.00% | 0.00% | 0.00% | 0.00% |
| 448 | 0.00% | 0.00% | 0.00% | 0.00% |
| 449 | 0.00% | 0.00% | 0.00% | 0.00% |

### Example 1D

### methyl 4-[(2R,4R)-4-amino-7-methoxy(3,3,4,5,6-²H₅)-3,4-dihydro-2H-chromen-2-yl](2,3,5-²H₃)benzoate

Methyl 4-[(2R,4E)-4-[(benzyloxy)imino]-7-methoxy(3,3,5,6-²H₄)-3,4-dihydro-2H-chromen-2-yl](2,3,5-²H₃)benzoate (3 g, 6.19 mmol), 10 wt % (dry basis) of 5% Pt/C (365 mg) and acetic acid-d₄ (45 mL) were charged to a Parr reactor. The reactor was sealed, and pressure purged with argon (4×100 psig) and then D₂ (4×100 psig). The reactor was then pressurized with D₂ (50 psig) and stirred overnight at 23° C. The reaction mixture was filtered through a 150-mL PE-fritted filter packed with diatomaceous earth (5 g) and rinsed with acetic acid (30 mL). The combined filtrate was concentrated to dryness in vacuo. A 4:1 mixture of methyl tert-butyl ether/heptanes (38 mL) was added to the residue, and the resulting slurry was heated to 62° C. and stirred. Acetic acid (12 mL) was added to dissolve the solids, and the mixture was heated to 70° C. and stirred for 15 minutes. A 3 M solution of HCl in cyclopentyl methyl ether (CPME), 7.2 mL) was added dropwise to precipitate a solid. The mixture was stirred for an additional 30 minutes at 70° C., and cooled to room temperature. The solids were collected by filtration and rinsed with ice-cold methyl tert-butyl ether (45 mL). The solid was then dried in a vacuum oven to obtain 1.94 g of beige colored solid. The solids were then charged to a reactor with a 2:1 mixture of CH₃CN/water (21 mL). The mixture was heated to 70° C., stirred for 1 hour, and cooled to room temperature. The mixture was stirred for 14 hours, the white solids were collected by filtration, and rinsed with methyl tert-butyl ether (6 mL). The solid was dried in a vacuum oven for 8 hours at 70° C. to afford the title compound as the hydrochloride salt in >99.9% ee. The isolated solid showed incorporation of 7.3 D-atoms by APCI-LC/MS.

Mass fragment data for native and labeled:

Native Substrate

| m/z | abund |
|---|---|
| 297 | 100.00% |
| 298 | 20.80% |
| 299 | 2.80% |
| 300 | 0.20% |
| 301 | 0.00% |
| 302 | 0.00% |
| 303 | 0.90% |
| 304 | 0.20% |
| 305 | 0.00% |
| 306 | 0.00% |
| 307 | 0.00% |

Obs Sample

| | | normalized | normalized | |
|---|---|---|---|---|
| m/z | obs abund | obs | calc | (obs-calc) |
| 297 | 0.00% | 0.00% | 0.00% | 0.00% |
| 298 | 0.00% | 0.00% | 0.00% | 0.00% |
| 299 | 0.00% | 0.00% | 0.00% | 0.00% |
| 300 | 0.00% | 0.00% | 0.00% | 0.00% |
| 301 | 12.40% | 12.40% | 12.40% | 0.00% |
| 302 | 31.70% | 31.70% | 31.71% | −0.01% |
| 303 | 67.10% | 67.10% | 67.08% | 0.02% |
| 304 | 100.00% | 100.00% | 100.00% | 0.00% |
| 305 | 94.00% | 94.00% | 93.99% | 0.01% |
| 306 | 59.30% | 59.30% | 59.30% | 0.00% |
| 307 | 30.50% | 30.50% | 30.54% | −0.04% |
| 308 | 13.70% | 13.70% | 13.09% | 0.61% |
| 309 | 0.00% | 0.00% | 2.78% | −2.78% |
| 310 | 0.00% | 0.00% | 1.15% | −1.15% |
| 311 | 0.00% | 0.00% | 0.86% | −0.86% |
| 312 | 0.00% | 0.00% | 0.52% | −0.52% |
| 313 | 0.00% | 0.00% | 0.26% | −0.26% |
| 314 | 0.00% | 0.00% | 0.11% | −0.11% |
| 315 | 0.00% | 0.00% | 0.01% | −0.01% |
| 316 | 0.00% | 0.00% | 0.00% | 0.00% |
| 317 | 0.00% | 0.00% | 0.00% | 0.00% |
| 318 | 0.00% | 0.00% | 0.00% | 0.00% |
| 319 | 0.00% | 0.00% | 0.00% | 0.00% |
| 320 | 0.00% | 0.00% | 0.00% | 0.00% |
| 321 | 0.00% | 0.00% | 0.00% | 0.00% |
| 322 | 0.00% | 0.00% | 0.00% | 0.00% |
| 323 | 0.00% | 0.00% | 0.00% | 0.00% |
| 324 | 0.00% | 0.00% | 0.00% | 0.00% |
| 325 | 0.00% | 0.00% | 0.00% | 0.00% |
| 326 | 0.00% | 0.00% | 0.00% | 0.00% |
| 327 | 0.00% | 0.00% | 0.00% | 0.00% |
| 328 | 0.00% | 0.00% | 0.00% | 0.00% |

### Example 1E

### methyl 4-[(2R,4R)-4-({[1-(2,2-difluoro-1,3-benzodioxol-5-yl)cyclopropyl]carbonyl}amino)-7-methoxy(3,3,4,5,6-²H₅)-3,4-dihydro-2H-chromen-2-yl](2,3,5-²H₃)benzoate

Methyl 4-[(2R,4R)-4-amino-7-methoxy(3,3,4,5,6-²H₅)-3,4-dihydro-2H-chromen-2-yl](2,3,5-²H₃)benzoate (1.28 g, 3.66 mmol), 1-(2,2-difluorobenzo[d][1,3]dioxol-5-yl)cyclopropane-1-carboxylic acid (0.916 g, 3.76 mmol), and O-(7-azabenzotriazol-1-yl)-N,N,N′,N′-tetramethyluronium hexafluorophosphate (HATU, 1.49 g, 3.93 mmol) were charged to a 50 mL round-bottom flask equipped with a magnetic stir bar, and the mixture was cooled to 0° C. Ethyl acetate (15 mL) was added. N-Ethyl-N-isopropylpropan-2-amine (DIPEA) (1.62 g, 12.5 mmol) was dissolved in ethyl acetate (3.8 mL) in a separate vial. The DIPEA solution in ethyl acetate was charged to the 50 mL round-bottom flask and stirred for 5 hours. The reaction mixture was filtered using a Buchner funnel and washed with ethyl acetate (10 mL). The organic layer was washed with 20 mL of 10% aqueous NaHCO₃ solution and brine (6×10 mL), dried over anhydrous sodium sulfate, filtered and concentrated in vacuo. Ethyl acetate (5.75 mL) was added to the oil obtained after concentration. Heptanes (35 mL) were added dropwise and the resulting solid was stirred overnight. The white solids were collected by filtration, washed with heptanes (2×17 mL), and dried in a vacuum oven at 45° C. to afford 1.51 g (2.76 mmol, 77%) of the title compound. The isolated solid showed incorporation of 7.6 D-atoms by APCI-LC/MS.

Mass fragment data for native and labeled:

Native Substrate

| m/z | abund |
|---|---|
| 536 | 100.00% |
| 537 | 33.80% |
| 538 | 6.90% |
| 539 | 1.30% |
| 545 | 0.30% |
| 541 | 0.30% |
| 542 | 0.00% |
| 543 | 0.30% |
| 544 | 0.00% |
| 545 | 1.60% |
| 546 | 0.00% |

Obs Sample

| | | normalized | normalized | |
|---|---|---|---|---|
| m/z | obs abund | obs | calc | (obs-calc) |
| 536 | 0.10% | 0.10% | 0.11% | −0.01% |
| 537 | 1.00% | 1.00% | 0.93% | 0.07% |
| 538 | 0.10% | 0.10% | 0.31% | −0.21% |
| 539 | 0.50% | 0.50% | 0.50% | 0.00% |
| 540 | 6.40% | 6.40% | 6.40% | 0.00% |
| 541 | 25.50% | 25.50% | 25.50% | 0.00% |
| 542 | 60.10% | 60.10% | 60.11% | −0.01% |
| 543 | 100.00% | 100.00% | 100.00% | 0.00% |
| 544 | 96.80% | 96.80% | 96.81% | −0.01% |
| 545 | 74.20% | 74.20% | 74.19% | 0.01% |
| 546 | 43.40% | 43.40% | 43.40% | 0.00% |
| 547 | 21.00% | 21.00% | 20.99% | 0.01% |
| 548 | 10.90% | 10.90% | 10.90% | 0.00% |
| 549 | 5.50% | 5.50% | 5.49% | 0.01% |
| 550 | 2.30% | 2.30% | 2.35% | −0.05% |
| 551 | 2.10% | 2.10% | 1.71% | 0.39% |
| 552 | 0.50% | 0.50% | 1.61% | −1.11% |
| 553 | 1.20% | 1.20% | 1.16% | 0.04% |
| 554 | 0.00% | 0.00% | 0.77% | −0.77% |
| 555 | 0.00% | 0.00% | 0.37% | −0.37% |
| 556 | 0.00% | 0.00% | 0.15% | −0.15% |
| 557 | 0.00% | 0.00% | 0.08% | −0.08% |
| 558 | 0.00% | 0.00% | 0.04% | −0.04% |
| 559 | 0.00% | 0.00% | 0.00% | 0.00% |
| 560 | 0.00% | 0.00% | 0.00% | 0.00% |
| 561 | 0.00% | 0.00% | 0.00% | 0.00% |
| 562 | 0.00% | 0.00% | 0.00% | 0.00% |
| 563 | 0.00% | 0.00% | 0.00% | 0.00% |
| 564 | 0.00% | 0.00% | 0.00% | 0.00% |
| 565 | 0.00% | 0.00% | 0.00% | 0.00% |
| 566 | 0.00% | 0.00% | 0.00% | 0.00% |
| 567 | 0.00% | 0.00% | 0.00% | 0.00% |

### Example 1F

### methyl 4-[(2R,4R)-4-({[1-(2,2-difluoro-1,3-benzodioxol-5-yl)cyclopropyl]carbonyl}amino)-7-hydroxy(3,3,4,5,6-²H₅)-3,4-dihydro-2H-chromen-2-yl](2,3,5-²H₃)benzoate

Methyl 4-[(2R,4R)-4-({[1-(2,2-difluoro-1,3-benzodioxol-5-yl)cyclopropyl]carbonyl}amino)-7-methoxy(3,3,4,5,6-²H₅)-3,4-dihydro-2H-chromen-2-yl](2,3,5-²H₃)benzoate (1.47 g, 2.69 mmol) was charged to a 50-mL jacketed flask equipped with a magnetic stir bar and purged with nitrogen. Dichloromethane (15 mL) was added under an atmosphere of nitrogen, and the resulting solution was cooled to −20° C. BCl₃ solution in dichloromethane (1 M) (8 mL, 8 mmol) was added dropwise to the reactor, and the reaction mixture was stirred for 30 minutes at −20° C. The reaction mixture was poured into ice-cold water (15 mL), and the mixture was allowed to warm to the room temperature. The resulting solution was concentrated under vacuum, methyl tert-butyl ether (15 mL) was added, and the slurry was mixed for 30 minutes. White solids precipitated out and were removed by filtration. The solids were rinsed with methyl tert-butyl ether (15 mL). The combined organic layer was taken into a separatory funnel, and the aqueous layer was discarded. The organic layer was washed with brine (20 mL), filtered through silica gel, washed with methyl tert-butyl ether (2×15 mL), and concentrated to dryness to obtain the title compound (1.32 g, 2.47 mmol, 92%).

### Example 1G

### 4-[(2R,4R)-4-({[1-(2,2-difluoro-1,3-benzodioxol-5-yl)cyclopropyl]carbonyl}amino)-7-(difluoromethoxy)(3,3,4,5,6-²H₅)-3,4-dihydro-2H-chromen-2-yl](3,5-²H₂)benzoic acid

Methyl 4-[(2R,4R)-4-({[1-(2,2-difluoro-1,3-benzodioxol-5-yl)cyclopropyl]carbonyl}amino)-7-hydroxy(3,3,4,5,6-²H₅)-3,4-dihydro-2H-chromen-2-yl](2,3,5-²H₃)benzoate (1.28 g, 2.42 mmol), and MeCN (5 mL) were charged to a 40 mL reaction vial, and the solution was put under a positive atmosphere of nitrogen and cooled down to −20° C. A 4 M solution of aqueous KOH (48.3 mmol) was added dropwise, followed by the addition of diethyl (bromodifluoromethyl)phosphonate (1.13 g, 1.75 mmol). The reaction mixture was allowed to warm to the room temperature over 30 minutes. Methanol (6 mL) was added, and the solution was heated to 40° C. and stirred for 1 hour. The reaction mixture was transferred to a separatory funnel, diluted with isopropyl acetate (12 mL), and the aqueous layer was rejected. The organic layer was transferred to another 40 mL reaction vial, and 2 M aqueous HCl (10 mL) was added. The mixture was heated to 40° C., stirred for 30 minutes, and cooled down to the room temperature. The reaction mixture was transferred to a separatory funnel, and washed with 2 M aqueous KOH (3×10 mL), 2 M aqueous HCl (10 mL), and brine (10 mL). The organic layer was dried over anhydrous sodium sulfate, filtered and concentrated in vacuo to obtain an oil. The crude material was purified by silica gel column chromatography using ethyl acetate/heptanes as the eluent. The product containing fractions were concentrated in vacuo to obtain a white solid, and the residue was dried overnight in a vacuum oven at 45° C. to obtain title compound (0.46 g, 0.81 mmol, 33%). The isolated solid showed incorporation of 6.5 D-atoms by APCI-LCMS. ¹H NMR (400 MHz, DMSO-d₆) δ ppm 13.0 (br, 1H), 7.97 (m, 1.2H), 7.54 (m, 0.34H), 7.42 (d, 1H), 7.32 (d, 1H), 7.26 (br, 1H), 7.22 (m, 1H), 7.20 (t, J=72 Hz, 1H), 7.10 (m, 0.01H), 6.75 (m, 0.51H), 6.68 (m, 0.68H), 5.42 (m, 0.85H), 5.40 (m, 0.08H), 2.05 (m, 0.39H), 1.52-1.49 (m, 0.95 H), 1.41-1.37 (m, 0.97H), 1.06 (m, 2H). The ¹H NMR signal at 7.54 ppm indicated greater than 80% deuterium incorporation at the two deuterated aryl positions in the acid containing aryl ring. The ¹H NMR signal at 7.10 ppm indicated greater than 95% deuterium incorporation in the aryl ring containing the —OCHF₂ group. The peak at 6.75 ppm indicated 50% deuterium incorporation in the aryl ring containing the —OCHF₂ group. The ¹H NMR signal at 5.40 ppm indicated greater than 90% deuterium incorporation at the benzylic carbon attached to the nitrogen atom. The ¹H NMR signal at 2.05 ppm min indicates greater than 80% incorporation of deuterium atoms on the carbon atom adjacent to the benzylic position. The peak at 7.97 ppm indicated 40% incorporation of deuterium atoms on the carbon atoms adjacent to the CO₂H functional group.

Mass fragment data for native and labeled:

Native Substrate

| m/z | abund |
|---|---|
| 558 | 100.00% |
| 559 | 29.70% |
| 560 | 5.90% |
| 561 | 0.90% |
| 562 | 0.10% |
| 563 | 0.00% |
| 564 | 0.00% |
| 565 | 0.00% |
| 566 | 0.00% |
| 567 | 0.12% |
| 568 | 0.00% |

Obs Sample

| | | normalized | normalized | |
|---|---|---|---|---|
| m/z | obs abund | obs | calc | (obs-calc) |
| 558 | 0.10% | 0.10% | 0.10% | 0.00% |
| 559 | 0.10% | 0.10% | 0.10% | 0.00% |
| 560 | 0.60% | 0.60% | 0.60% | 0.00% |
| 561 | 3.40% | 3.40% | 3.40% | 0.00% |
| 562 | 17.60% | 17.60% | 17.60% | 0.00% |
| 563 | 48.20% | 48.20% | 48.20% | 0.00% |
| 564 | 92.50% | 92.50% | 92.50% | 0.00% |
| 565 | 100.00% | 100.00% | 100.00% | 0.00% |
| 566 | 68.60% | 68.60% | 68.60% | 0.00% |
| 567 | 35.60% | 35.60% | 35.60% | 0.00% |
| 568 | 13.50% | 13.50% | 13.50% | 0.00% |
| 569 | 3.30% | 3.30% | 3.30% | 0.00% |
| 570 | 0.60% | 0.60% | 0.59% | 0.01% |
| 571 | 0.00% | 0.00% | 0.10% | −0.10% |
| 572 | 0.00% | 0.00% | 0.06% | −0.06% |
| 573 | 0.00% | 0.00% | 0.09% | −0.09% |
| 574 | 0.00% | 0.00% | 0.09% | −0.09% |
| 575 | 0.00% | 0.00% | 0.05% | −0.05% |
| 576 | 0.00% | 0.00% | 0.02% | −0.02% |
| 577 | 0.00% | 0.00% | 0.01% | −0.01% |
| 578 | 0.00% | 0.00% | 0.00% | 0.00% |
| 579 | 0.00% | 0.00% | 0.00% | 0.00% |
| 580 | 0.00% | 0.00% | 0.00% | 0.00% |
| 581 | 0.00% | 0.00% | 0.00% | 0.00% |
| 582 | 0.00% | 0.00% | 0.00% | 0.00% |
| 583 | 0.00% | 0.00% | 0.00% | 0.00% |
| 584 | 0.00% | 0.00% | 0.00% | 0.00% |
| 585 | 0.00% | 0.00% | 0.00% | 0.00% |
| 586 | 0.00% | 0.00% | 0.00% | 0.00% |
| 587 | 0.00% | 0.00% | 0.00% | 0.00% |
| 588 | 0.00% | 0.00% | 0.00% | 0.00% |
| 589 | 0.00% | 0.00% | 0.00% | 0.00% |

### Example 2

### 4-[(2R,4R)-4-({[1-(2,2-difluoro-1,3-benzodioxol-5-yl)(2H₄)cyclopropyl]carbonyl}amino)-7-(difluoromethoxy)-3,4-dihydro-2H-chromen-2-yl]benzoic acid

### Example 2A

### methyl 4-{(2R)-4-oxo-7-[(propan-2-yl)oxy]-3,4-dihydro-2H-1-benzopyran-2-yl}benzoate

A 20 mL vial was charged with bis(2,2,2-trifluoroacetoxy)palladium (0.197 g, 0.594 mmol), (S)-4-(tert-butyl)-2-(pyridin-2-yl)-4,5-dihydrooxazole (0.146 g, 0.713 mmol), ammonium hexafluorophosphate(V) (0.581 g, 3.56 mmol) and (4-(methoxycarbonyl)phenyl)boronic acid (2.138 g, 11.88 mmol). The mixture was stirred in dichloroethane (5 mL) for 5 minutes. To the suspension was added 7-(difluoromethoxy)-4H-chromen-4-one (1.26 g, 5.94 mmol) and water (0.256 mL, 14.19 mmol) and the sides of the vial were washed with additional dichloroethane (5 mL). The vial was capped and the mixture was stirred at 60° C. overnight. The mixture was filtered through a plug of silica gel and diatomaceous earth and was eluted with ethyl acetate. The solvent was removed in vacuo and the crude residue was chromatographed on a 40 g silica gel cartridge, eluting with a gradient of 5-50% ethyl acetate/heptanes to provide the title compound (860 mg, 2.469 mmol, 41.6% yield). MS (APCI+) m/z 349.3 (M+H)⁺.

### Example 2B

### methyl 4-[(2R)-4-[(benzyloxy)imino]-7-(difluoromethoxy)-3,4-dihydro-2H-1-benzopyran-2-yl]benzoate

A 40 mL vial was charged with Example 2A (577 mg, 1.657 mmol) and pyridine (5.7 mL). O-Benzylhydroxylamine hydrochloride (278 mg, 1.739 mmol) was added and the reaction was heated at 50° C. for over 1 hour. The reaction mixture was cooled to room temperature and concentrated. The residue was taken into ethyl acetate. The mixture was washed with saturated aqueous NH₄Cl, 1 M aqueous HCl, and brine. The organic layer was concentrated and the residue (2.1 mg) was dissolved in ethyl acetate followed by dropwise addition of 5 mL heptanes. The resulting solid was collected by filtration and washed with heptanes. The solid was dried under vacuum to provide the title compound (430.7 mg). ¹H NMR (400 MHz, CDCl₃) δ ppm 8.03-7.96 (m, 2H), 7.89-7.82 (m, 1H), 7.47-7.39 (m, 2H), 7.35-7.18 (m, 5H), 6.71-6.60 (m, 2H), 6.44 (t, J=73.5 Hz, 1H), 5.14 (d, J=1.2 Hz, 2H), 5.06 (dd, J=12.2, 3.2 Hz, 1H), 3.90-3.83 (m, 3H), 3.43 (ddd, J=17.4, 3.3, 1.0 Hz, 1H), 2.62 (ddd, J=17.3, 12.2, 1.1 Hz, 1H).

### Example 2C

### methyl 4-[(2R,4R)-4-amino-7-(difluoromethoxy)-3,4-dihydro-2H-1-benzopyran-2-yl]cyclohexane-1-carboxylate hydrochloride

A 50 mL Parr reactor was charged with Example 2B (1.55 g, 3.42 mmol) and platinum (IV) oxide (0.124 g, 0.305 mmol). The solids were suspended in methanol (23 mL) and 2,2,2-trifluoroacetic acid (2.62 mL, 34.2 mmol). The reactor was sealed and purged with argon (60 psig, 4 times) and then with H₂ (100 psig, 4 times). The reactor was pressurized to 150 psig of H₂, warmed to 50° C. and stirred for 6 hours. The mixture was cooled to 23° C. The pressure in the reactor was vented carefully. The contents of the reactor were then purged with argon (60 psig, 3 times). The slurry was filtered to remove the catalyst, rinsed with ethyl acetate (2×30 mL), washed with 10 wt % aqueous K₃PO₄ (18 mL, 3 times), and concentrated (to about 12 mL). HCl (4 M in cyclopentyl methyl ether, 3.42 mL, 13.7 mmol) was added. The mixture was stirred for three hours and concentrated to provide the title compound. ¹H NMR (400 MHz, CDCl₃) δ ppm 7.42 (d, J=8.0 Hz, 2H), 6.69-6.66 (m, 2H), 6.55 (m, 2H), 6.47 (br s, 1H), 6.29 (s, 0.3H), 4.06-4.01 (m, 2H), 3.97-3.89 (m, 2H), 3.70 (s, 3H), 3.68 (s, 3H), 2.32-2.06 (m, 10H), 1.88-1.38 (m, 22H), 1.30-1.11 (m, 4H). ¹³C NMR (100 MHz, CDCl₃) δ ppm 176.3, 175.6, 155.7, 151.0, 127.8, 124.8, 116.0 (t, J=257 Hz), 111.4, 107.3, 79.8, 79.1, 51.5, 47.0, 46.9, 43.2, 41.5, 41.1, 39.6, 36.4, 28.6, 27.4, 27.0, 26.5, 24.9, 24.7. ¹⁹F NMR (376 MHz, CDCl₃) δ ppm −76.0, −80.7.

### Example 2D

### ethyl 2-cyano-2-(2,2-difluorobenzo [d][1,3]dioxol-5-yl)acetate

A 250 mL round bottom flask with stir bar was charged with toluene (100 mL), and the solvent was sparged with N₂ for one hour. Trisodium phosphate (20.75 g, 127 mmol) was added, followed by bis(dibenzylideneacetone)palladium (0.970 g, 1.688 mmol). A solution of tri-tert-butylphosphine (0.683 g, 3.38 mmol) was added over 10 minutes via syringe, and the suspension was stirred for 50 minutes at 25° C. 5-Bromo-2,2-difluorobenzo[d][1,3]dioxole (10 g, 42.2 mmol) was added, and after stirring for 50 minutes at 25° C., ethyl 2-cyanoacetate (9.55 g, 84 mmol) was added over 5 minutes, followed by the addition of 1.5 mL of water. The reaction was heated at 75° C. for 12 hours. Three additional vials were set up as described above. After completion of the reaction, all four reaction mixtures were combined. The reaction was cooled to 25° C. and filtered over diatomaceous earth. The filtrate was concentrated, and the residue was purified by column chromatography on silica gel (petroleum ether/ethyl acetate=5/1) to provide the title compound (24.5 g, 48.5% yield). ¹H NMR (400 MHz, DMSO-d₆) δ ppm 1.15-1.23 (m, 3H) 4.13-4.29 (m, 2H) 7.30 (dd, J=8.38, 1.76 Hz, 1H) 7.46-7.55 (m, 2H).

### Example 2E

### 2-(2,2-difluorobenzo[d][1,3]dioxol-5-yl)acetonitrile

To the solution of Example 2D (8 g, 29.7 mmol) in dimethyl sulfoxide (80 mL) was added 3 M aqueous HCl (80 mL) and the reaction was heated at 110° C. for 5 hours. Two additional vials were set up as described above. After completion of the reaction, all three reaction mixtures were combined. The reaction was extracted with methyl tert-butyl ether (2×500 mL) and the combined organics were washed with brine, dried over Na₂SO₄, filtered and concentrated to give a residue, which was purified by column chromatography on silica gel (petroleum ether/ethyl acetate=5/1) to provide the title compound (12 g, 68.3% yield). ¹H NMR (400 MHz, DMSO-d₆) δ ppm 3.24-3.56 (m, 1H) 4.02 (s, 2H) 7.20 (d, J=8.38 Hz, 1H) 7.34-7.43 (m, 2H).

### Example 2F

### 1-(2,2-difluoro-1,3-benzodioxol-5-yl)(²H₄)cyclopropanecarbonitrile

To a solution of Example 2E (4 g, 20.29 mmol) in methyl tert-butyl ether (40 mL) was added tetrabutylammonium bromide (0.327 g, 1.015 mmol), followed by degassed 50% aqueous NaOH (40 mL) over 20 minutes at 0° C. BrCD₂CD₂Br (1,2-dibromo(2H₄)ethane, 6.62 g, 34.5 mmol) was added via cannula over 5 minutes. The reaction was stirred at 25° C. for 12 hours. Two additional vials were set up as described above. After completion of the reaction, all three reaction mixtures were combined. The reaction was poured into ice water, maintaining a temperature of about 15° C. The mixture was extracted with methyl tert-butyl ether (2×200 mL) and the organic phase was dried over Na₂SO₄, filtered and concentrated to give a residue, which was purified by column chromatography on silica gel (eluted with petroleum/ethyl acetate=5/1) to provide the title compound (6 g, 43.4% yield). ¹H NMR (400 MHz, DMSO-d₆) δ ppm 7.28 (dd, J=8.38, 1.76 Hz, 1H) 7.36-7.48 (m, 1H).

### Example 2G

### 1-(2,2-difluoro-1,3-benzodioxol-5-yl)(²H₄)cyclopropanecarboxylic acid

To a solution of Example 2F (3 g, 13.20 mmol) in ethanol (30 mL) was slowly added a solution of NaOH (5.28 g, 132 mmol) in water (30 mL), keeping the internal temperature below 25° C. After the addition, the reaction was stirred at 80° C. for 12 hours. Two additional vials were set up as described above. After completion of the reaction, all three reaction mixtures were combined. The ethanol was removed under reduced pressure and 6 M aqueous HCl (100 mL) was added dropwise to the residue, at which time a precipitate formed. The solid was collected, washed with water and dried under high vacuum to provide title compound (2.9 g, 87% yield). ¹H NMR (400 MHz, DMSO-d₆) δ ppm 7.16 (dd, J=8.38, 1.76 Hz, 1H) 7.30 (d, J=8.16 Hz, 1H) 7.39 (d, J=1.76 Hz, 1H) 12.41 (br s, 1H).

### Example 2H

### 4-[(2R,4R)-4-({[1-(2,2-difluoro-1,3-benzodioxol-5-yl) (²H₄)cyclopropyl]carbonyl}amino)-7-(difluoromethoxy)-3,4-dihydro-2H-chromen-2-yl]benzoic acid

To Example 2G (76 mg, 0.309 mmol) in N,N-dimethylformamide (2 mL) was added HATU (N-[(dimethylamino)-1H-1,2,3-triazolo-[4,5-b]pyridin-1-ylmethylene]-N-methylmethanaminium hexafluorophosphate N-oxide, 176 mg, 0.464 mmol). The mixture was stirred for 5 minutes, and methyl 4-((2R,4R)-4-amino-7-(difluoromethoxy)chroman-2-yl)benzoate (Example 2C, 150 mg, 0.309 mmol) was added, followed by addition of N-ethyl-N-isopropylpropan-2-amine (0.215 mL, 1.236 mmol). The mixture was stirred at room temperature for two hours. The mixture was loaded onto a 24 g silica gel cartridge, eluting with 0-50% ethyl acetate in heptane, to provide the coupled ester. The ester was concentrated and dissolved in methanol (2 mL) and 6 N aqueous LiOH (0.5 mL). The mixture was stirred at 50° C. for three hours. The pH was adjusted to 0-1 by adding 2 N aqueous HCl. The solvent was removed under pressure and the residue was purified via chromatography on a 24 g silica gel cartridge eluting with 9:1 ethyl acetate/methanol in heptane at a 5-60% gradient to provide the title compound (130 mg, 74.7% yield). ¹H NMR (501 MHz, Chloroform-d) δ ppm 8.14-8.08 (m, 2H), 7.51-7.47 (m, 2H), 7.26 (s, 1H), 7.13-7.00 (m, 4H), 6.74-6.68 (m, 2H), 6.40 (d, J=73.7 Hz, 1H), 5.48 (td, J=9.9, 6.1 Hz, 1H), 5.36 (d, J=8.8 Hz, 1H), 5.27 (dd, J=11.2, 1.9 Hz, 1H), 2.52 (ddd, J=13.4, 6.1, 2.1 Hz, 1H), 1.79 (dt, J=13.4, 11.2 Hz, 1H). MS (ESI−) m/z 564 (M+H)⁺.

### Determination of Biological Activity

### Cellular Assays

### Cell Surface Expression-Horse Radish Peroxidase (CSE-HRP) Assay:

A cellular assay for measuring the F508delCFTR cell surface expression after correction with test compounds was developed in human lung derived epithelial cell line (CFBE41o-) (Veit G et al, (2012) Mol Biol Cell. 23(21): 4188-4202). This was achieved by expressing the F508delCFTR mutation along with a horseradish peroxidase (HRP) in the fourth exofacial loop and then measuring the HRP activity using luminescence readout from these cells, CFBE41o-F508delCFTR-HRP, that were incubated overnight with the test corrector compounds. Briefly, for this primary assay, the CFBE41o-F508delCFTR-HRP cells were plated in 384-well plates (Greiner Bio-one; Cat 781080) at 4,000 cells/well along with 0.5 μg/mL doxycycline to induce the F508delCFTR-HRP expression and further incubated at 37° C., 5% CO₂ for 72 hours. The test compounds were then added at the required concentrations and further incubated for 18-24 hours at 33° C. The highest concentration tested was 20 M with an 8-point concentration response curve using a 3-fold dilution. Three replicate plates were run to determine one EC₅₀. All plates contained negative controls (dimethyl sulfoxide, DMSO) and positive controls (3 μM of 3-[(2R,4R)-4-({[1-(2,2-difluoro-1,3-benzodioxol-5-yl)cyclopropyl]carbonyl}amino)-7-methoxy-3,4-dihydro-2H-chromen-2-yl]benzoic acid) as well as on-plate concentration response of the positive control. Post incubation, the plates were washed 5 x times with Dulbecco's phosphate buffered saline (DPBS), followed by the addition of the HRP substrate, luminol (50 μL), and measuring the HRP activity using luminescence readout on EnVision® Multilabel Plate Reader (Perkin Elmer; product number 2104-0010). The raw counts from the experiment are analyzed using Accelrys® Assay Explorer v3.3.

Z′ greater than 0.5 was used as passing quality control criteria for the plates.

The Z′ is defined as:

1−[3*SD_{Positive Control}+3*SD_{Negative Control}/Absolute(Mean_{Positive Control}−Mean_{Negative Control})]

wherein “SD” is standard deviation.

The % activity measured at each of the 8 test concentrations of the test compound was normalized to the on-plate positive control using the following formula:

% activity=[(test compound response−DMSO response)/(positive control response−DMSO response)]*100

The maximum % activity achieved for the test compound at any tested concentration is presented in Table 1 along with the EC₅₀ calculated using the following general sigmoidal curve with variable Hill slope equation (described as Model 42 in the Accelrys® Assay Explorer v3.3 software):

y=(α−d)/(1+(x/c)\hat{ }b)+d

General sigmoidal curve with concentration, response, top, bottom, EC₅₀ and Hill slope.

This model describes a sigmoidal curve with an adjustable baseline, a. The equation can be used to fit curves where response is either increasing or decreasing with respect to the independent variable, “x”.

“x” is a concentration of drug under test.
“y” is the response.
“a” is the maximum response, and “d” is the minimum response
“c” is the inflection point (EC₅₀) for the curve. That is, “y” is halfway between the lower and upper asymptotes when x=c.
“b” is the slope-factor or Hill coefficient. The sign of b is positive when the response increases with increasing dose and is negative when the response decreases with increasing dose (inhibition).

TABLE 1
CSE-HRP data

| Example | EC₅₀ (μM) | Maximum % activity (%) |
|---|---|---|
| 1 | 0.027 | 145 |

### Transepithelial Clamp Circuit on Human Bronchial Epithelial Cells Conductance Assay:

A cell based assay using the primary human bronchial epithelial cells (hBE) was used as a secondary assay to test novel F508delCFTR correctors for their activity on primary hBE cells with F508del/F508del CFTR mutation. The assay used a TECC-24 (Transepithelial Clamp Circuit for 24 wells) instrument that measures the functionality of the mutated channel by measuring the equivalent short circuit current (Ieq) generated by the polarized epithelial cells. The instrument works by measuring the transepithelial potential difference (Vt) and transepithelial resistance (Rt) in an open circuit format, and the Ieq is calculated by using Ohms law (Ieq=Vt/Rt). The assay was run in a 24-well format and all 24-wells were measured at the same time point giving a higher throughput for this assay.

Primary human bronchial epithelial (hBE) cells from F508del/F508delCFTR patients were expanded from 1×10⁶ to 250×10⁶ cells (Neuberger T, Burton B, Clark H and VanGoor F; Cystic Fibrosis, Methods in Mole Biol 741; eds. Amaral M D and Kunzelmann K, 2011). For this purpose, cells isolated from CF patients with the homozygous mutation were seeded onto 24 well Corning (Cat #3378) filter plates that were coated with 3T3 conditioned media and grown at an air-liquid interface for 35 days using an Ultroser® G supplemented differentiation media. Apical surface mucus was removed 72 hours before the experiment using 3 mM dithiothreitol (DTT) in phosphate buffered saline (PBS). The apical surface was washed again 24 hours before the experiment using PBS. The cells were incubated with the desired dose response of the corrector compounds 18-24 hours at 37° C., 5% CO₂. The corrector compounds are only added on the basolateral side of the epithelial cells.

On the day of measuring the corrector activity on the TECC, the cells were switched into a bicarbonate and serum free F-12 Coon's medium and allowed to equilibrate for 90 minutes in a CO₂ free incubator. At the time of measurement, the apical and basolateral sides of the filter were bathed with the F-12 Coon's modification media (with 20 mM 4-(2-hydroxyethyl)-1-piperazineethanesulfonic acid (HEPES), pH 7.4 (using 1 M tris(hydroxymethyl)aminomethane (Tris)), and the measurements were made at 36.5° C. Transepithelial voltage (Vt) and transepithelial resistance (Rt) were measured using a 24 channel transepithelial current clamp (TECC-24). Current responses to the sequential addition of benzamil (apical 6 μM addition; for inhibiting epithelial ENaC channel), forskolin (apical and basolateral 10 μM addition; for activating the CFTR channel), control potentiator (N-(3-carbamoyl-5,5,7,7-tetramethyl-4,7-dihydro-5H-thieno[2,3-c]pyran-2-yl)-1H-pyrazole-5-carboxamide; apical and basolateral 1 μM addition; for potentiating the CFTR channel) and bumetanide (basolateral 20 μM addition; for inhibiting the Na:2Cl:K co-transporter, an indirect measure of inhibiting the Cl⁻ secretion driven by CFTR channel) were measured.

All plates contained negative controls (dimethyl sulfoxide, DMSO) which coupled with the control potentiator (N-(3-carbamoyl-5,5,7,7-tetramethyl-4,7-dihydro-5H-thieno[2,3-c]pyran-2-yl)-1H-pyrazole-5-carboxamide) sets the null response and positive controls (0.15 μM) of 4-[(2R,4R)-4-({[1-(2,2-difluoro-1,3-benzodioxol-5-yl)cyclopropyl]carbonyl}amino)-7-(difluoromethoxy)-3,4-dihydro-2H-chromen-2-yl]benzoic acid coupled with the control potentiator sets the 100% response to measure the correction of the mutated CFTR channel. The maximum percent activity is reported relative to the positive control value.

The % activity measured at each of the 6 test concentrations of the test compound was normalized to the on-plate positive control using the following formula:

% activity=[(test compound response−DMSO response)/(positive control response−DMSO response)]*100

The following log(agonist) vs response using a four parameters variable slope was used to calculate EC₅₀ (4 PL in Prism v 5 software):

F(x)=D+(A−D)/(1+(x/C)\hat{ }B)

### Where:

“x” is a concentration of drug under test.
“F(x)” is the response.
“A” is the maximum response, and “D” is the minimum response
“C” is the inflection point (EC₅₀) for the curve. That is, “F(x)” is halfway between the lower and upper asymptotes when x=C.
“B” is the slope-factor or Hill coefficient. The sign of B is positive when the response increases with increasing dose and is negative when the response decreases with increasing dose (inhibition).

The maximum percent activity and EC₅₀ values for tested corrector compounds are presented in Table 2.

TABLE 2
hBE-TECC data

| Example | EC₅₀ (μM) | Maximum % activity (%) |
|---|---|---|
| 1 | 0.013 | 96 |

What is claimed:

1. A compound of Formula (I), or a pharmaceutically acceptable salt thereof, 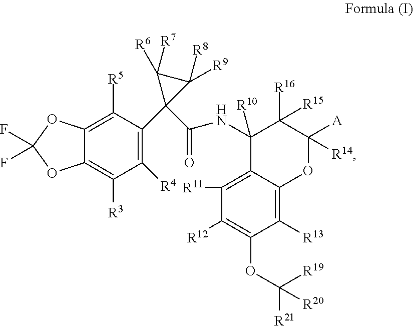
wherein
R¹ and R² are each independently hydrogen, deuterium, or fluorine;
A is 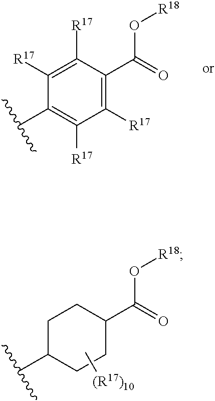
R³, R⁴, R⁵, R⁶, R⁷, R⁸, R⁹, R¹⁰, R¹¹, R¹², R¹³, R¹⁴, R¹⁵, R¹⁶, and R¹⁸ are each independently hydrogen or deuterium;
R¹⁷, at each occurrence, is independently hydrogen or deuterium; and
R¹⁹, R²⁰, and R²¹ are each independently hydrogen, deuterium, or fluorine;
provided that, at least one of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ is deuterium.

2. The compound of claim 1, or a pharmaceutically acceptable salt thereof, wherein A is

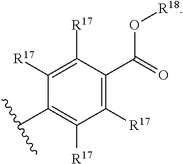

3. The compound of claim 1, or a pharmaceutically acceptable salt thereof, wherein A is

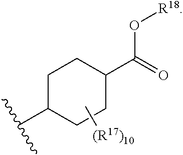

4. The compound of claim 1, or a pharmaceutically acceptable salt thereof, wherein R¹ and R² are each fluorine.

5. The compound of claim 1, or a pharmaceutically acceptable salt thereof, wherein R¹⁹, R²⁰, and R²¹ are each hydrogen.

6. The compound of claim 1, or a pharmaceutically acceptable salt thereof, wherein two of R¹⁹, R²⁰, and R²¹ are fluorine.

7. A compound of Formula (VI), or a pharmaceutically acceptable salt thereof, 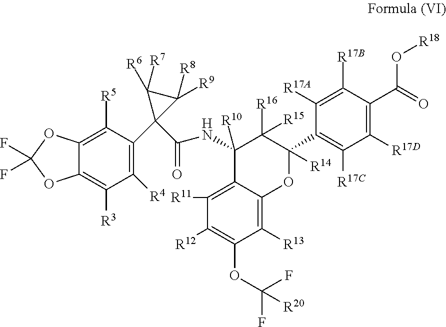
wherein
R³, R⁴, R⁵, R⁶, R⁷, R⁸, R⁹, R¹⁰, R¹¹, R¹², R¹³, R¹⁴, R¹⁵, R¹⁶, and R¹⁸ are each independently hydrogen or deuterium;
R^{17A}, R^{17B}, R^{17C}, R^{17D} are each independently hydrogen or deuterium; and
R²⁰ is independently hydrogen, deuterium, or fluorine;
provided that, at least one of R³, R⁴, R⁵, R⁶, R⁷, R⁸, R⁹, R¹⁰, R¹¹, R¹², R¹³, R¹⁴, R¹⁵, R¹⁶, R^{17A}, R^{17B}, R^{17C}, R^{17D}, R¹⁸, and R²⁰ is deuterium.

8. 4-[(2R,4R)-4-({[1-(2,2-difluoro-1,3-benzodioxol-5-yl)cyclopropyl]carbonyl}amino)-7-(difluoromethoxy)(3,3,4,5,6-²H₅)-3,4-dihydro-2H-chromen-2-yl](3,5-²H₂)benzoic acid, 4-[(2R,4R)-4-({[1-(2,2-difluoro-1,3-benzodioxol-5-yl)(²H₄)cyclopropyl]carbonyl}amino)-7-(difluoromethoxy)-3,4-dihydro-2H-chromen-2-yl]benzoic acid, or a pharmaceutically acceptable salt thereof.

9. A pharmaceutical composition comprising a therapeutically effective amount of a compound of Formula (I) according to claim 1, or a pharmaceutically acceptable salt thereof, in combination with a pharmaceutically acceptable carrier.

10. A compound of claim 1, or a pharmaceutically acceptable salt thereof, or a pharmaceutical composition of claim 9, for use in medicine.

11. A compound of claim 1, or a pharmaceutically acceptable salt thereof, or a pharmaceutical composition of claim 9, for use in the treatment of cystic fibrosis.

12. A pharmaceutical composition comprising a compound of claim 1, or a pharmaceutically acceptable salt thereof, one potentiator, and one or more additional correctors.

13. A pharmaceutical composition comprising a compound of claim 1, or a pharmaceutically acceptable salt thereof, and one or more additional therapeutic agents.

14. The pharmaceutical composition of claim 13 wherein the additional therapeutic agents are selected from the group consisting of CFTR modulators and CFTR amplifiers.

15. The pharmaceutical composition of claim 13 wherein the additional therapeutic agents are CFTR modulators.
